SEQUENCE LISTING (22 sequences)

SEQ ID NO: 1 - DNA, 101 bp, Homo sapiens
  modified_base (51)..(51): a, c, t, g, unknown or other
tctttgtctgcttttgatttgcctttctgtcctctgtcttccattcaacanctagctctatattctcatgggatcatcatgtctacttttagcagtcagtt

SEQ ID NO: 2 - DNA, 101 bp, Homo sapiens
  modified_base (51)..(51): a, c, t, g, unknown or other
tgggtcctaatccagtgactggtatttttattagaagggaggaatttgctnggcgtggaatttgctaggcatggtggctcatgcctgtaatcccagctact

SEQ ID NO: 3 - DNA, 101 bp, Homo sapiens
  modified_base (51)..(51): a, c, t, g, unknown or other
ggtactcactgacactgtctgttgactcttttggccttttcagattctggngcaacagagtgggtgacgagggggcccaggccctggctgaagccttgggt

SEQ ID NO: 4 - DNA, 102 bp, Homo sapiens
  modified_base (52)..(52): a, c, t, g, unknown or other
ttaggcaatggagtaaggaaaaaagaccattggatttcaagagaggacactngagtctttctgggtgacttggaaatgtcccttgtcctctcagggttttga

SEQ ID NO: 5 - DNA, 101 bp, Homo sapiens
  modified_base (51)..(51): a, c, t, g, unknown or other
tctcccctccctgttgtcagctacctcatatgttctctaatctctgtctcngtgccctcaaagacccccctgaaaatagaaatattactgctcattggtta

SEQ ID NO: 6 - DNA, 101 bp, Homo sapiens
  modified_base (51)..(51): a, c, t, g, unknown or other
ttcagtatgaaagaggtggttttctttccccttactgcttgcagcttaagngtacaaaaatgtttgtacctttacctaaattataatggtcctaaggtggt

SEQ ID NO: 7 - DNA, 101 bp, Homo sapiens
  modified_base (51)..(51): a, c, t, g, unknown or other
agtgttaagcactcagccaggtccgtctggctctggcacagggctctggcnaggtactattcgtcctctgatgtcatgcttccagaattgcagaatgtttg

SEQ ID NO: 8 - DNA, 101 bp, Homo sapiens
  modified_base (51)..(51): a, c, t, g, unknown or other
taacctccatttgcaaaggtatgaaggtcataaaagtcatgacatatgtcnggaacaatctaatcacagcaactagtaaggaaaggcacttgattggacaa

SEQ ID NO: 9 - DNA, 101 bp, Homo sapiens
  modified_base (51)..(51): a, c, t, g, unknown or other
gccacaggtaaggggaagctaataatgaaatgaaaaccaaatgcatgcccncgttctatgcaactactgactttcttcctacaaatgttagttcaagccta

SEQ ID NO: 10 - DNA, 103 bp, Homo sapiens
  modified_base (53)..(53): a, c, t, g, unknown or other
atttttcttttttgtttgtgtcctttcctggttttggtattagggtgacacangttcatagaattaactgggaagagttccttctttatcttgtggaatcgtt

SEQ ID NO: 11 - DNA, 101 bp, Homo sapiens
  modified_base (51)..(51): a, c, t, g, unknown or other
acagacaaaccttgaagtctctgtggcttgacagaagggagtttatttctnactcatgtcactaaccagtgcaggtggtcctggtcaggacatcttccaca

SEQ ID NO: 12 - DNA, 101 bp, Homo sapiens
tctttgtctgcttttgatttgcctttctgtcctctgtcttccattcaacarctagctctatattctcatgggatcatcatgtctacttttagcagtcagtt

SEQ ID NO: 13 - DNA, 101 bp, Homo sapiens
tgggtcctaatccagtgactggtatttttattagaagggaggaatttgctrggcgtggaatttgctaggcatggtggctcatgcctgtaatcccagctact

SEQ ID NO: 14 - DNA, 101 bp, Homo sapiens
ggtactcactgacactgtctgttgactcttttggccttttcagattctggbgcaacagagtgggtgacgagggggcccaggccctggctgaagccttgggt

SEQ ID NO: 15 - DNA, 102 bp, Homo sapiens
ttaggcaatggagtaaggaaaaaagaccattggatttcaagagaggacactbgagtctttctgggtgacttggaaatgtcccttgtcctctcagggttttga

SEQ ID NO: 16 - DNA, 101 bp, Homo sapiens
tctcccctccctgttgtcagctacctcatatgttctctaatctctgtctcygtgccctcaaagacccccctgaaaatagaaatattactgctcattggtta

SEQ ID NO: 17 - DNA, 101 bp, Homo sapiens
ttcagtatgaaagaggtggttttctttccccttactgcttgcagcttaagrgtacaaaaatgtttgtacctttacctaaattataatggtcctaaggtggt

SEQ ID NO: 18 - DNA, 101 bp, Homo sapiens
agtgttaagcactcagccaggtccgtctggctctggcacagggctctggcvaggtactattcgtcctctgatgtcatgcttccagaattgcagaatgtttg

SEQ ID NO: 19 - DNA, 101 bp, Homo sapiens
taacctccatttgcaaaggtatgaaggtcataaaagtcatgacatatgtcrggaacaatctaatcacagcaactagtaaggaaaggcacttgattggacaa

SEQ ID NO: 20 - DNA, 101 bp, Homo sapiens
gccacaggtaaggggaagctaataatgaaatgaaaaccaaatgcatgcccmcgttctatgcaactactgactttcttcctacaaatgttagttcaagccta

SEQ ID NO: 21 - DNA, 103 bp, Homo sapiens
atttttcttttttgtttgtgtcctttcctggttttggtattagggtgacacargttcatagaattaactgggaagagttccttctttatcttgtggaatcgtt

SEQ ID NO: 22 - DNA, 101 bp, Homo sapiens
acagacaaaccttgaagtctctgtggcttgacagaagggagtttatttctyactcatgtcactaaccagtgcaggtggtcctggtcaggacatcttccaca

(19) United States
(12) Patent Application Publication
(10) Pub. No.: US 2022/0273665 A1
(43) Pub. Date: Sep. 1, 2022

(54) METHODS OF TREATING INFLAMMATORY BOWEL DISEASES THAT TARGET RIPK2

(71) Applicant: CEDARS-SINAI MEDICAL CENTER, Los Angeles, CA (US)

(72) Inventors: Dermot MCGOVERN, Los Angeles, CA (US); Dalin LI, Los Angeles, CA (US); Janine BILSBOROUGH, Los Angeles, CA (US)

(21) Appl. No.: 17/357,625

(22) Filed: Jun. 24, 2021

Related U.S. Application Data
(63) Continuation of application No. PCT/US2019/067786, filed on Dec. 20, 2019.
(60) Provisional application No. 62/786,221, filed on Dec. 28, 2018.

(51) Int. Cl. A61K 31/5377; A61K 31/5025; A61K 31/44; A61K 31/517; A61P 1/00

(52) U.S. Cl. CPC A61K 31/5377; A61K 31/5025; A61P 1/00; A61K 31/517; A61K 31/44

(57) ABSTRACT

Described herein are methods, systems, compositions, and kits useful for the diagnosis and/or treatment of inflammatory bowel diseases, including Crohn's disease and ulcerative colitis in a subject, with an antagonist of RIPK2 activity or expression. The present disclosure relates to methods and systems for identifying and stratifying patients suitable for treatment with the antagonist to RIPK2 activity or expression, as described herein.

CROSS-REFERENCE

This application is continuation of International Application No. PCT/US2019/067786 filed Dec. 20, 2019, which claims the benefit of U.S. Provisional Application Ser. No.62/786,221, filed Dec. 28, 2018, which is incorporated herein by reference in its entirety.

### STATEMENT REGARDING FEDERALLY SPONSORED RESEARCH OR DEVELOPMENT

This invention was made with government support under Grant No. DK062413 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy created Jun. 24, 2021 is named 56884-747_301_SL.txt and is 6,099 bytes in size.

### SUMMARY

Inflammatory bowel diseases (IBD) is a heterogeneous group of chronic, relapsing inflammatory disorders of the gastrointestinal (GI) tract affecting more than 3 million adults in the United States, according to the most recent Centers for Disease and Prevention (CDC) survey. The two most common manifestations of IBD are Crohn's disease (CD) and ulcerative colitis (UC). Each of these forms of IBD has various subclinical phenotypes that manifest in certain IBD patients. For example, the chronic inflammation of the GI tract caused by CD and UC leads to the formation of scar tissue (fibrosis) and stenosis (fibrostenosis) in the intestinal wall in some IBD patients, that is largely unresponsive to current therapeutic interventions. For these patients endoscopic or surgical treatment is often the only treatment available.

IBD, including CD and UC, is characterized by an uncontrolled activity of the immune response within the intestinal mucosal, which depends on genetic susceptibility to developing the IBD, subclinical phenotypes of IBD, as well as to various stimuli related to IBD pathogenesis (e.g., intestinal microbiome). Genome Wide Association Studies (GWAS) have enabled scientists to identify genetic variants in certain IBD susceptibility gene loci useful for the selection of IBD patients for treatment with targeted therapeutic strategies, and identifying drugable targets in the development of novel therapies.

Few treatment options are available to patients that suffer from IBD. Current therapeutic regimens include one or more of anti-inflammatory medication (e.g., corticosteroids) and immunomodulatory therapy (e.g., anti-TNF therapy). However, nearly half of all patients treated with an anti-TNF therapy do not respond to the induction of the therapy, or experience a loss of response to the treatment after a period of time, during which, disease severity has progressed significantly. Therefore, there remains a significant need for targeted and effective treatment options that respond to the underlying immunopathogenesis of IBD.

Genome Wide Association Studies (GWAS) have enabled scientists to identify genetic variations in certain genes and gene loci that are associated with IBD, including CD and UC, and subclinical phenotypes of IBD, CD, and UC. GWAS compare the allele frequency in a given population of a particular genetic variation between unrelated cases and controls, each case representing a patient with IBD and each control representing an individual without IBD. GWAS, the Immunochip, and their meta-analysis have enabled the discovery of over 200 single nucleotide polymorphisms (SNPs) associated with IBD, including CD and UC. In some embodiments, disclosed herein, are methods, systems, compositions, and kits for the treatment, diagnosis, and prognosis of IBD in subjects who carry various genotypes (e.g., SNPs) located at IBD risk genes involve in the NOD2/CARD15 and autophagy pathways.

Aspects disclosed herein provide methods of treating or preventing a disease or condition in a subject, the method comprising administering a modulator of Receptor Interacting Serine/Threonine Kinase 2 (RIPK2) activity or expression to the subject, provided a genotype is detected in a sample obtained the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods of reducing or ablating activity or expression of Receptor Interacting Serine/Threonine Kinase 2 (RIPK2) in a subject, the method comprising administering a modulator of RIPK2 to the subject, provided a genotype is detected in a sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10¹⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods of treating or preventing a disease or condition in a subject, the method comprising: obtaining a sample from a subject; detecting a presence or an absence of a genotype in the sample obtained from the subject; and administering to the subject a modulator of Receptor Interacting Serine/Threonine Kinase 2 (RIPK2) activity or expression to the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰ about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods of reducing or ablating activity or expression of Receptor Interacting Serine/Threonine Kinase 2 (RIPK2) in a subject, the method comprising: obtaining a sample from a subject; detecting a presence or an absence of a genotype in the sample obtained from the subject; and administering to the subject a modulator of RIPK2 activity or expression to the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises one or more SNPs provided in any one of Tables 1-4.

Aspects disclosed herein provide methods of diagnosing a disease or condition in a subject, the method comprising: obtaining a sample from a subject; detecting a presence or an absence of a genotype in the sample obtained from the subject; and diagnosing the disease or condition in the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the methods further comprise administering to the subject a modulator or RIPK2. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4. Aspects disclosed herein provide methods of determining whether a subject is at risk for developing a disease or condition, in a subject, the method comprising: obtaining a sample from a subject; detecting a presence or an absence of a genotype in the sample obtained from the subject; and determining the subject is at risk for developing the disease or condition, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the methods further comprise administering to the subject a modulator or RIPK2. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻¹⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods of determining whether a subject is suitable for treatment of a disease or condition with a modulator of Receptor Interacting Serine/Threonine Kinase 2 (RIPK2) activity or expression, the method comprising: obtaining a sample from a subject; detecting a presence or an absence of a genotype in the sample obtained from the subject; and determining the subject is suitable for treatment of the disease or condition with a modulator of RIPK2, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the methods further comprise administering to the subject a modulator or RIPK2. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods for processing or analyzing a sample obtained from a subject, the method comprising: obtaining a sample from a subject; subjecting the sample to an assay by sequencing, genotype array, and/or nucleic acid amplification, to yield a data set comprising data corresponding to a presence or an absence of a genotype; in a programmed computer, inputting said data from (b) to a trained algorithm to determine whether the subject is at risk of developing, a disease or disorder, wherein the trained algorithm is trained with a plurality of training samples, and wherein said sample is independent of said plurality of training samples; and electronically outputting a report comprising the determination for the subject. In some embodiments, (c) comprises calculating a polygenic risk score (PRS), and the PRS comprises a normalized weighted sum of a number of risk alleles within the genotype present in the subject with weights proportional to a beta value of association between the genotype with the disease or condition. In some embodiments, the data set of (b) further comprises data corresponding to a presence or an absence of a surrogate genotype, provided an absence of a genotype is detected. In some embodiments, the surrogate genotype is in linkage disequilibrium with the absent genotype as determined by an r² value of at least about, 0.8, about0.85, about 0.90, about 0.95, or about 1.0. In some embodiments, the report is configured to display the determination of the subject on a user interface of an electronic device. In some embodiments, the electronic device comprises a personal electronic device belonging to the subject. In some embodiments, the methods further comprise administering to the subject a modulator or RIPK2, provided the subject is determined to be at risk of having, or developing, the disease or condition. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more. In some embodiments, the genotype comprises one or more SNPs provided in any one of Tables 1-4.

Aspects disclosed herein provide methods for processing or analyzing a sample obtained from a subject, the method comprising: obtaining a sample from a subject; subjecting the sample to an assay by sequencing, genotype array, and/or nucleic acid amplification, to yield a data set comprising data corresponding to a presence or an absence of a genotype; in a programmed computer, inputting said data from (b) to a trained algorithm to determine a likelihood that the subject is suitable for treatment of a disease or disorder with an antagonist of RIPK2, wherein the trained algorithm is trained with a plurality of training samples, and wherein said sample is independent of said plurality of training samples; and electronically outputting a report comprising the determination for the subject. In some embodiments, (c) comprises calculating a polygenic risk score (PRS), and the PRS comprises a normalized weighted sum of a number of risk alleles within the genotype present in the subject with weights proportional to a beta value of association between the genotype with the disease or condition. In some embodiments, the data set of (b) further comprises data corresponding to a presence or an absence of a surrogate genotype, provided an absence of a genotype is detected. In some embodiments, the surrogate genotype is in linkage disequilibrium with the absent genotype as determined by an r² value of at least about, 0.8, about 0.85, about 0.90, about 0.95, or about 1.0. In some embodiments, the report is configured to display the determination of the subject on a user interface of an electronic device. In some embodiments, the electronic device comprises a personal electronic device belonging to the subject. In some embodiments, the methods further comprise administering to the subject a modulator or RIPK2, provided the subject is determined to be at risk of having, or developing, the disease or condition. In some embodiments, the modulator of RIPK2 activity or expression comprises an antagonist or a partial antagonist of RIPK2. In some embodiments, the antagonist or partial antagonist comprises an antibody or antigen-binding fragment, or small molecule. In some embodiments, the antagonist or partial antagonist comprises a RIPK2 inhibitor effective to bind to the ATP binding pocket of the RIPK2 kinase domain. In some embodiments, the antagonist or partial antagonist comprises a type II RIPK2 inhibitor effective to displace a RIPK2 kinase activation segment. In some embodiments, the antagonist or partial antagonist comprises a an ATP-competitive type I RIPK2 inhibitor. In some embodiments, the antagonist or partial antagonist comprises a RIPK2 inhibitor effective to inhibit RIPK2 ubiquitination. In some embodiments, the antagonist or partial antagonist comprises a RIPK2 inhibitor effective to inhibit RIPK2 autophosphorylation. In some embodiments, the antagonist or partial antagonist comprises a RIPK2 inhibitor effective to block NOD-dependent tumor necrosis factor production without affecting lipopolysaccharide-dependent pathways. In some embodiments, the antagonist or partial antagonist comprises a RIPK2 inhibitor effective to bind an allosteric site of RIPK2. In some embodiments, the antagonist or partial antagonist comprises ponatinib, sorafenib, regorafenib, gefitinib, or erlotinib. In some embodiments, the antagonist or partial antagonist comprises a structure of Formula I. In some embodiments, the antagonist or partial antagonist comprises a structure of Formula II. In some embodiments, the antagonist or partial antagonist comprises a structure of Formula III. In some embodiments, the antagonist or partial antagonist comprises a structure of Formula IV. In some embodiments, the antagonist or partial antagonist comprises a structure of Formula V. In some embodiments, the antagonist or partial antagonist comprises a structure of any one of Formulas VI-X. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) at rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” allele. In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some embodiments, the SNP at rs11564258 is within SEQ ID NO: 1. In some embodiments, the SNP at rs2357623 is within SEQ ID NO: 2. In some embodiments, the SNP at rs2066845 is within SEQ ID NO: 3. In some embodiments, the SNP at rs5743289 is within s SEQ ID NO: 4. In some embodiments, the SNP at rs72796367 is within SEQ ID NO: 5. In some embodiments, the SNP at rs6752107 is within SEQ ID NO: 6. In some embodiments, the SNP at rs12994997 is within SEQ ID NO: 7. In some embodiments, the SNP at rs11741861 is within SEQ ID NO: 8. In some embodiments, the SNP at rs9494844 is within SEQ ID NO: 9. In some embodiments, the SNP at rs6918329 is within SEQ ID NO: 10. In some embodiments, the SNP at rs7404095 is within SEQ ID NO: 11. In some embodiments, LD is defined by an r² value of at least 0.80, 0.85, 0.90, 0.95, r 1.0. In some embodiments, the genotype comprises one or more single nucleotide polymorphisms (SNPs) located at a gene comprising Leucine Rich Repeat Kinase (LRRK). In some embodiments, the genotype comprises one or more single nucleotide polymorphisms (SNPs) located at a gene comprising Nucleotide Binding Oligomerization Domain Containing 2 (NOD2). In some embodiments, the genotype comprises one or more single nucleotide polymorphisms (SNPs) located at a gene comprising Autophagy Related 16 Like 1 (ATG16L1). In some embodiments, the genotype comprises one or more single nucleotide polymorphisms (SNPs) located at a gene comprising Zinc Finger Protein 300 (ZNF300). In some embodiments, the genotype comprises one or more single nucleotide polymorphisms (SNPs) located at a gene comprising Oligodendrocyte Transcription Factor 3 (OLIG3). In some embodiments, the genotype comprises one or more single nucleotide polymorphisms (SNPs) located at a gene comprising Protein Kinase C Beta (PRKCB1). In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰ about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, stricturing and penetrating, disease phenotypes. In some embodiments, the genotype comprises one or more SNPs in linkage disequilibrium with rs5743289 as determined by an r² value of at least about 0.80, about 0.85, about 0.90, about 0.95, or about 1.0. In some embodiments, the genotype comprises at least about 1 single nucleotide polymorphism (SNP), about 2 SNPs, about 3 SNPs, about 4 SNPs, about 5 SNPs, about 6 SNPs, about 7 SNPs, about 8 SNPs, about 9 SNPs, about 10 SNPs, about 11 SNPs, or more.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Terminologies

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “patient” and “subject” encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some cases, the subject is a patient that is diagnosed with a disease or disorder described herein. In some cases, the subject is suspected of having the disease or disorder but is not necessarily diagnosed.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “genetic variant” as used herein refers to an aberration in (e.g., a mutation), or of (e.g., copy number variation), a nucleic acid sequence, as compared to the nucleic acid sequence in a reference population. In some embodiments, the genetic variant is common in the reference population. In some embodiments, the genetic variant is rare in the reference population.

The term, “genotype” as disclosed herein, refers to the chemical composition of polynucleotide sequences within the genome of an individual. In some embodiments, the genotype comprises single nucleotide variant (SNV), a single nucleotide polymorphism (SNP), or and indel (insertion or deletion, of a nucleobase within a polynucleotide sequence). In some embodiments, a genotype for a particular SNV, SNP, or indel is heterozygous. In some embodiments, a genotype for a particular SNV, SNP, or indel is homozygous.

The terms, “single nucleotide polymorphism” or “SNP,” as disclosed herein, refer to a variation in a single nucleotide within a polynucleotide sequence. The variation of an SNP may have multiple different forms. A single form of an SNP is referred to as an “allele.” An SNP can be mono-, bi-, tri, or tetra-allelic. An SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP , in some cases, is observed in at least 1% of a given population. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNV (nucleotide position) within the dbSNP sequence, the position of which is always with reference to 5′ length of the sequence plus 1. In some embodiments, a SNP is defined as the genomic position in a reference genome and the allele change. In some embodiments, the SNP is defined as the genomic position identified with a non-nucleic acid letter code (e.g., IUPAC) in a sequence disclosed herein, such as in SEQ ID NOS: 1-22.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the dbSNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] in a sequence disclosed herein.

“Haplotype” as used herein, encompasses a group of one or more genotypes, SNVs, SNPs, or indels, which tend to be inherited together in a reference population. In some embodiments, a haplotype comprises particular SNVs, SNPs, or indels, and any SNV, SNP, or indel in linkage disequilibrium therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r² value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, or gene expression product. In some embodiments, a biomarker comprises a cytokine (e.g., IL-1α, IL-1β, IL-2, IL-3. IL-4, IL-5, IL-6, IL-8, IL-9, IL-10, IL-13, IL-17, IL-17F, IL-22, TNF-α, TNF-β, IFN-α1/-α2, IFN-β, IFN-γ, TNFSF superfamily: TNF, TL1A, FasL, LIGHT, TRAIL, and TWEAK). In some embodiments, a biomarker comprises a cell type (e.g., Natural Killer (NK) cells, T cells, Effector T cells (Teff), Regulatory T cells (Treg) B cells, T helper (Th) cells, cluster of differentiation (CD) cells, innate lymphoid cells (ILC), antigen-presenting cells (APC), monocytes Paneth cells, granulocytes, dendritic cells, and macrophages).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-I2 antibody, and anti-Cbirl flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject. Non-limiting examples of bacteria associated with IBD includes strains, sub-strains, and enterotypes of enterobacteriacease, pasteurellaceae, fusobacteriacease, neisseriaceae, veillonellaceae, gemellaceae, bacteriodales, clostridales, erysipelotrichaeceae, bifidobacteriaceae bacteroides, faecalibacterium, roseburia, blautia, ruminococcus, coprococcus, streptococcus, dorea, blautia, ruminococcus, lactobacillus, enterococcus, streptococcus, escherichia coli, fusobacterium nucleatum, haemophilus parainfluenzae (pasteurellaceae), veillonella parvula, eikenella corrodens (neisseriaceae), and gemella moribillum, bacteroides vulgatus, bacteroides caccae, bifidobacterium bifidum, bifidobacterium longum, bifidobacterium adolescentis, bifidobacterium dentum, blautia hansenii, ruminococcus gnavus, clostridium nexile, faecalibacterium prausnitzii, ruminoccus torques, clostridium bolteae, eubacterium rectale, roseburia intestinalis, coprococcus comes, actinomyces, lactococcus, roseburia, streptococcus, blautia, dialister, desulfovibrio, escherichia, lactobacillus, coprococcus, clostridium, bifidobacterium, klebsiella, granulicatella, eubacterium, anaerostipes, parabacteroides, coprobacillus, gordonibacter, collinsella, bacteroides, faecalibacterium, anaerotruncus, alistipes, haemophilus, anaerococcus, veillonella, arevotella, akkermansia, bilophila, sutterella, eggerthella, holdemania, gemella, peptoniphilus, rothia, enterococcus, pediococcus, citrobacter, odoribacter, enterobacteria, fusobacterium, and proteus. Non-limiting examples of viruses associated with IBD include picovirinae, lactococcus phage, cellulophaga phage, bacteroides phage, C2 like virus, enterococcus phage, caudivurales, cellulophaga phage, phiCD119 like virus, croceibacter phage, clostridium phage, spounavirinae, riemerella phage, lambda like virus, bacillus phage, terenvirinae, lactobacillus phage, enterobacteria phage, thermoanaerobacterium phage, strepcoccus phage, and pseudomonas phage. Non-limiting examples of fungi genera associated with IBD includes Malassezia, Cladosporium, Aureobasidium, Fusarium, Candida, Pichia, Saccharomyces, and Escherichia.

The term, “transcriptomic risk signature,” or “transcriptomic signature,” as disclosed herein, refers to two or more biomarkers comprising transcribed polynucleotides (e.g., RNA, cDNA) whose presence, level, or activity, is indicative of a phenomenon (e.g., phenotypic expression or activity; disease, condition, subclinical phenotype of a disease or condition, infection; or environmental stimuli). In some embodiments, the biomarkers comprise non-protein coding oligonucleotide sequence. In some embodiments, the biomarkers comprise protein coding oligonucleotide sequence, such as mRNA or cDNA.

The term, “transcriptomic risk profile,” or “transcriptomic profile,” as disclosed herein, refers to the particular level of expression or activity of a transcriptomic risk signature or transcriptomic signature in a subject at a point in time.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The term “anti-tumor necrosis factor (TNF) non-response,” or “anti-TNF non-response,” as used herein, refers to a subject not responding to the induction of an anti-TNF therapy (primary non-response), or loss of response during maintenance after a successful induction of the anti-TNF therapy (secondary loss of response). In some embodiments, the induction of the anti-TNF therapy comprises 1, 2, 3, 4, or 5, doses of the therapy. In some embodiments, loss of response is characterized by a reappearance of symptoms consistent with a flare after an initial response to the anti-TNF therapy.

Where substituent groups are specified by their conventional chemical formulae, written from left to right, they equally encompass the chemically identical substituents that result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and includes mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂-], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl. An alkoxy is an alkyl attached to the remainder of the molecule via an oxygen linker (—O—).

As used herein, an “alkenyl” group refers to an aliphatic carbon group that contains 2-8 (e.g., 2-8, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-8, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

As used herein, an “amido” encompasses both “aminocarbonyl” and “carbonylamino”. These terms when used alone or in connection with another group refer to an amido group such as —N(R^{X})—C(O)—R^{Y} or —C(O)—N(R^{X})₂, when used terminally, and —C(O)N(R^{X})— or —N(R^{X})C(O)— when used internally, wherein RX and RY are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

As used herein, a “carbamoyl” group refers to a group having the structure —O—CO—NR^{X}R^{Y} or —NR^{X}—CO—O—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogens. For instance, the term haloalkyl includes the group —CF₃.

As used herein, a “mercapto” group refers to —SH.

As used herein, a “sulfo” group refers to —SO₃H or SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —SR^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)R^{X} when used terminally and S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—SO—R^{X} or —SOOR^{X}, when used terminally and —O—S(O)— or —S(O)—O— when used internally, where R^{X} has been defined above.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CONR^{Y}R^{Z} and a “thiourea” group refers to the structure —NR^{X}—CS—NR^{Y}R^{Z} when used terminally and NR^{X}—CONR^{Y}—or —NR^{X}—CSNR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C (═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ— where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, and R₃, and other variables contained in formulae described herein encompass specific groups, such as alkyl and aryl. Unless otherwise noted, each of the specific groups for the variables R₁, R₂, and R₃, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this disclosure are those combinations that result in the formation of stable or chemically feasible compounds.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-i soxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol [symbol] denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

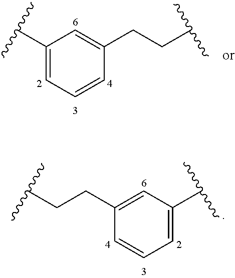 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ′NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, —halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula —T−C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)₄—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X— (C″R″R′″)_{d}—, where s and d are independently integers of from 0 to 3, and X′ is —O—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.
(c) A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

### Methods

### Disease or Condition

Aspects disclosed herein provide methods of treating, diagnosing, determining a risk of developing, or monitoring, a disease or condition. In some cases, the disease or condition comprises an inflammatory disease, fibrostenotic disease, and/or fibrotic disease. Non-limiting examples of inflammatory diseases include diseases of the gastrointestinal (GI) tract, liver, gallbladder, and joints. In some cases, the inflammatory disease inflammatory bowel disease (IBD), Crohn's disease (CD), or ulcerative colitis, systemic lupus erythematosus (SLE), or rheumatoid arthritis. A subject may suffer from fibrosis, fibrostenosis, or a fibrotic disease, either isolated or in combination with an inflammatory disease. An exemplary fibrotic disease is primary sclerosing cholangitis (PSC).

In some instances, the disease or condition is refractory, which refers a quality of the disease or condition such that there is an observed failure of a standard treatment to induce remission of a disease or condition. Non-limiting examples of refractory inflammatory disease include refractory Crohn's disease, and medically refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin. In some instances, the refractory disease or condition is characterized by an increase in colitis, inflammation, fibrosis, fibrostenosis, stricturing, penetrating, obstructive, or otherwise complicated, disease of the GI tract.

Subject

Disclosed herein, in some embodiments, are methods of treating, diagnosing, prognosing, or monitoring, a disease or condition in a subject. In some instances, the subject is a mammal. In some embodiments, the subject comprises a mouse, rat, guinea pig, rabbit, chimpanzee, or farm animal. In some instances, the subject is human. In some instances, the subject is diagnosed with the disease or condition disclosed herein. Non-limiting methods for diagnosis using existing indices and scoring systems include Crohn's Disease Activity Index (CDAI), Ulcerative Colitis Disease Activity Index (UCDAI), guidelines from American College of Gastroenterology (ACG) and European Crohn's and Colitis Organization (ECCO), patient-reported outcomes (PRO-2), Harvey-Bradshaw Index, Van Hess Index, Perianal Disease Activity Index (PDAI), Rachmilewitz score, Mayo score, Powell-Tuck index, Patient Simple Clinical Colitis Activity Index (P-SCCAI), Lichtiger index, Seo index, Inflammatory Bowel Disease Questionnaire (IBDQ), Manitoba IBD Index, Crohn's Disease Endoscopic Index of Severity (CDEIS), Simple Endoscopic Score for Crohn Disease (SES-CD), Lewis score (capsule endoscopy), Rutgeert's Score, and the Montreal Classification, and IBD questionnaire. In some instances, the subject is not diagnosed with the disease or condition. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). In further embodiments provided, the subject is, or is suspected of being, non-responsive to a standard treatment (e.g., anti-TNF alpha therapy, anti-a4-b7 therapy (vedolizumab), anti-IL12p40 therapy (ustekinumab), Thalidomide, or Cytoxin). In some cases, the subject is not responsive to the induction of said therapy. In some cases, the subject loses response to said standard treatment after a period of time during treatment.

Method of Detection

Disclosed herein, in some embodiments, are methods of detecting a presence, absence, or level, of a genotype or biomarker in a sample obtained from a subject. In some instances, the methods of detection disclosed herein are useful for the diagnosis, prognosis, monitoring of disease progression, selection for treatment, monitoring of treatment, and/or treatment of inflammatory bowel disease (e.g., Crohn's disease, ulcerative colitis, and the like) disclosed herein.

In some embodiments, methods of detecting a presence, absence, or level of a genotype or biomarker in the sample obtained from the subject involve detecting a nucleic acid sequence. In some cases, the nucleic acid sequence comprises deoxyribonucleic acid (DNA). In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, the genotype or biomarker is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genetic variants disclosed herein is used. In some instances, the nucleic acid probe specific to a SNP or SNV comprises a nucleic acid probe sequence sufficiently complementary to a risk or protective allele of interest, such that hybridization is specific to the risk or protective allele. In some instances, the nucleic acid probe specific to an indel comprises a nucleic acid probe sequence sufficiently complementary to an insertion of a nucleobase within a polynucleotide sequence flanking the insertion, such that hybridization is specific to the indel. In some instances, the nucleic acid probe specific to an indel comprises a probe sequence sufficiently complementary to a polynucleotide sequence flanking a deletion of a nucleobase within the polynucleotide sequence, such that hybridization is specific to the indel. In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

The present disclosure provides exemplary probes that are hybridizable to a target nucleic acid sequence comprising rs11564258, rs2357623, rs2066845, rs5743289, rs72796367, rs6752107, rs12994997, rs11741861, rs9494844, rs6918329, rs7404095. The present disclosure provides exemplary probes provided in SEQ ID NOS: 12-22, respectively. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 12. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 13. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 3. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 14. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 15. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 16. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 17. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 18. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 19. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 20. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 21. In some embodiments, the exemplary probe comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 22.

In some embodiments, the exemplary probes provided herein are specific to a risk allele within rs11564258, rs2357623, rs2066845, rs5743289, rs72796367, rs6752107, rs12994997, rs11741861, rs9494844, rs6918329, rs7404095. In some embodiments, the SNP at rs11564258 comprises an “A” or a “G” risk allele. In some embodiments, the SNP at rs2357623 comprises an “A” or a “G” risk allele. In some embodiments, the SNP at rs2066845 comprises a “G” or a “C” risk allele. In some embodiments, the SNP at rs5743289 comprises an “A” or a “G” risk allele. In some embodiments, the SNP at rs72796367 comprises a “G” or an “A” risk allele. In some embodiments, the SNP at rs6752107 comprises a “G” or an “A” risk allele. In some embodiments, the SNP at rs12994997 comprises a “G” or an “A” risk allele. In some embodiments, the SNP at rs11741861 comprises a “G” or an “A” In some embodiments, the SNP at rs9494844 comprises an “A” or a “C” risk allele. In some embodiments, the SNP at rs6918329 comprises a “G” or an “A” risk allele. In some embodiments, the SNP at rs7404095 comprises an “A” or a “G” risk allele. In some embodiments, methods disclosed herein detect at least 1 SNP, about 2 SNPs, at least about 3 SNPs, at least about 4 SNPs, at least about 5 SNPs, at least about 6 SNPs, at least about 7 SNPs, at least about 8 SNPs, at least about 9 SNPs, at least about 10 SNPs, at least about 11 SNPs, at least about 12 SNPs, at least about 13 SNPs, at least about 14 SNPs, at least about 15 SNPs, at least about 20 SNPs, at least about 25 SNPs, at least about 30 SNPs, at least about 40 SNPs, or at least about 50 SNPs.

In some embodiments, the one or more SNPs is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰. In some embodiments, the one or more SNPs is associated with a risk that the subject has, or will develop, a subclinical phenotype (e.g., stricturing and/or penetrating disease) of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰.

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-l-sulfonic acid (EDANS). Exemplary coumarins include, e.g., 3-phenyl-7-isocyanatocoumarin; acridines, such as 9-isothiocyanatoacridine and acridine orange; N-(p-(2-b enzoxazolyl)phenyl)maleimide; cyanines, such as, e.g., indodicarbocyanine 3 (Cy3), indodicarbocyanine 5 (Cy5), indodicarbocyanine 5.5 (Cy5 .5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7, 12,13,16,17-octahydro-inner salt (TR or Texas Red); or BODIPY™ dyes. In some cases, the probe comprises FAM as the dye label.

Disclosed herein, in some embodiments, a genotype or biomarker is detected by subjecting a sample obtained from the subject to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genetic risk variants disclosed herein. In some instances, the amplification assays requires primers. The nucleic acid sequence for the genetic risk variants and/or genes known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). Controlled robotic systems are useful for isolating and amplifying nucleic acids and can be used.

In some embodiments, detecting the biomarker or genotype of the subject comprises sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Disclosed herein, in some embodiments, genetic material is extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. A target protein may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. In some cases, the target protein comprises Receptor Interacting Serine/Threonine Kinase 2 (RIPK2), Leucine Rich Repeat Kinase (LRRK). Nucleotide Binding Oligomerization Domain Containing 2 (NOD2), Autophagy Related 16 Like 1 (ATG16L1), Zinc Finger Protein 300 (ZNF300), Oligodendrocyte Transcription Factor 3 (OLIG3), and/or Protein Kinase C Beta (PRKCB1). Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein, in some embodiments, are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), E. coli outer membrane porin protein C (OmpC), antibody to Pseudomonas fluorescens-associated sequence I2 (I2), and/or antibody to bacertail flagellin (Cbir1). In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suscpected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

Methods of Diagnosis and Prognosis

Disclosed herein, in some embodiments, are methods of diagnosing a disease or condition in a subject. In some cases, the disease or condition comprises an inflammatory disease, fibrostenotic disease, and/or fibrotic disease. Non-limiting examples of inflammatory diseases include diseases of the GI tract, liver, gallbladder, and joints. In some cases, the inflammatory disease IBD, CD, UC, systemic lupus erythematosus (SLE), or rheumatoid arthritis. In some embodiments, the disease or condition comprises fibrosis, fibrostenosis, or a fibrotic disease, either isolated or in combination with an inflammatory disease. An exemplary fibrotic disease is PSC. In some embodiments, a subtype of the disease or condition is diagnosed in the subject. Non-limiting examples of subtypes of IBD include, stricturing disease, penetrating disease, stricturing and penetrating disease, obstructive disease, refractory disease, or another complicated form of IBD. In some instances, the subject is diagnosed with, or predicted to develop, one disease or condition, two disease or conditions, three disease or conditions, or more.

Disclosed herein, in some embodiments, are methods of diagnosing a disease or condition in a subject, the method comprising: (a) obtaining a sample from a subject; (b) detecting a presence or an absence of a genotype in the sample obtained from the subject; and (c) diagnosing the disease or condition in the subject, provided the presence of the genotype is detected in the sample obtained from the subject. In some embodiments, the genotype is detected using one or more methods of detection, kits and/or compositions disclosed herein. In some embodiments, the subject is treated by administering a therapeutically effective amount of a therapeutic agent and/or additional agent disclosed herein to the subject, provided the subject is diagnosed with the disease or condition. In some embodiments, the therapeutic agent comprises an antagonist of RIPK2, such as those described herein.

Also disclosed herein, in some embodiments, are methods of diagnosing a disease or condition in a subject, the method comprising: (a) obtaining a sample from a subject; (b) detecting a presence or an absence of a genotype in the sample obtained from the subject; and (c) diagnosing the disease or condition in the subject, provided the presence of the genotype is detected in the sample obtained from the subject. In some embodiments, the genotype is detected using one or more methods of detection, kits and/or compositions disclosed herein. In some embodiments, the subject is treated by administering a therapeutically effective amount of a therapeutic agent and/or additional agent disclosed herein to the subject, provided the subject is predicted to develop the disease or condition. In some embodiments, the therapeutic agent comprises an antagonist of RIPK2, such as those described herein.

Also disclosed herein, in some embodiments, are methods of determining whether a subject is at risk for developing a disease or condition, in a subject, the method comprising: (a) obtaining a sample from a subject; (b) detecting a presence or an absence of a genotype in the sample obtained from the subject; and (c) determining the subject is at risk for developing the disease or condition, provided the presence of the genotype is detected in the sample obtained from the subject. In some embodiments, the genotype is detected using one or more methods of detection, kits and/or compositions disclosed herein. In some embodiments, the subject is treated by administering a therapeutically effective amount of a therapeutic agent and/or additional agent disclosed herein to the subject, provided the subject is predicted to develop the disease or condition. In some embodiments, the therapeutic agent comprises an antagonist of RIPK2, such as those described herein.

In some embodiments, the genotype described herein comprises a single nucleotide polymorphism (SNP). In some embodiments, the one or more SNPs are located at a gene comprising LRRK, NOD2, ATG16L1, SNF300, OLIG3, and/or PRKCB1. Non-limiting examples of SNPs located at the gene comprising LRRK, NOD2, ATG16L1, SNF300, OLIG3, and/or PRKCB1 LRRK, NOD2, ATG16L1, SNF300, OLIG3, and/or PRKCB1 include SNPs from any one of Tables 2-4. In some embodiments, the genotype comprises one or more SNPs selected from Table 1. In some embodiments, the SNPs located at the genes described herein are associated with a likelihood that a subject is suitable for treatment of a disease or disorder with an antagonist of RIPK2 activity of expression, such as those described herein.

In some embodiments, the one or more SNPs is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰. In some embodiments, the one or more SNPs is associated with a risk that the subject has, or will develop, a subclinical phenotype (e.g., stricturing and/or penetrating disease) of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰.

In some embodiments, the genotype comprises one or more SNPs in linkage disequilibrium with rs5743289 as determined by an r² value of at least about 0.80, about 0.85, about 0.90, about 0.95, or about 1.0. In some instances, the r² value is 0.80. In some instances, the r² value is 0.85. In some instances, the r² value is 0.90. In some instances, the r² value is 0.95.

In some instances, the r² value is 1.0. In some embodiments, the one or more SNPs comprises rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” risk allele. In some embodiments, the SNP at rs2357623 comprises an “A” or a “G” risk allele. In some embodiments, the SNP at rs2066845 comprises a “G” or a “C” risk allele. In some embodiments, the SNP at rs5743289 comprises an “A” or a “G” risk allele. In some embodiments, the SNP at rs72796367 comprises a “G” or an “A” risk allele. In some embodiments, the SNP at rs6752107 comprises a “G” or an “A” risk allele. In some embodiments, the SNP at rs12994997 comprises a “G” or an “A” risk allele. In some embodiments, the SNP at rs11741861 comprises a “G” or an “A” In some embodiments, the SNP at rs9494844 comprises an “A” or a “C” risk allele. In some embodiments, the SNP at rs6918329 comprises a “G” or an “A” risk allele. In some embodiments, the SNP at rs7404095 comprises an “A” or a “G” risk allele. In some instances, the SNP is within a polynucleotide sequence provided in SEQ ID NOS: 1-11.

Methods of Characterizing a Subtype of a Disease or Condition

Disclosed herein, in some embodiments, are methods of characterizing a disease or condition, or a subtype of a disease or condition. In some cases, the disease or condition comprises an inflammatory disease, fibrostenotic disease, and/or fibrotic disease. Non-limiting examples of inflammatory diseases include diseases of the GI tract, liver, gallbladder, and joints. In some cases, the inflammatory disease IBD, CD, UC, systemic lupus erythematosus (SLE), or rheumatoid arthritis. In some embodiments, the disease or condition comprises fibrosis, fibrostenosis, or a fibrotic disease, either isolated or in combination with an inflammatory disease. An exemplary fibrotic disease is PSC. Non-limiting examples of subtypes of IBD include, stricturing disease, penetrating disease, stricturing and penetrating disease, obstructive disease, refractory disease, or another complicated form of IBD.

Disclosed herein, in some embodiments, are methods of characterizing a disease a disease or condition, or a subtype of a disease or condition comprising: (a) obtaining a sample from a subject; (b) detecting a presence or an absence of a genotype in the sample obtained from the subject; and (c) characterizing the disease or condition as being stricturing, penetrating, or stricturing and penetrating disease, provided the presence, absence, or level of the genotype is detected in the sample obtained from the subject. In some embodiments, the genotype is detected using one or more methods of detection, kits and/or compositions disclosed herein. In some embodiments, the subject is treated by administering a therapeutically effective amount of a therapeutic agent and/or additional agent disclosed herein to the subject, provided the subject is disease or condition is characterized as stricturing, penetrating, and/or stricturing and penetrating disease.

In some embodiments, the genotype of the subject comprises one or more single nucleotide polymorphisms (SNPs). In some embodiments, systems described herein utilize at least 1 SNP, about 2 SNPs, at least about 3 SNPs, at least about 4 SNPs, at least about 5 SNPs, at least about 6 SNPs, at least about 7 SNPs, at least about 8 SNPs, at least about 9 SNPs, at least about 10 SNPs, at least about 11 SNPs, at least about 12 SNPs, at least about 13 SNPs, at least about 14 SNPs, at least about 15 SNPs, at least about 20 SNPs, at least about 25 SNPs, at least about 30 SNPs, at least about 40 SNPs, or at least about 50 SNPs. In some embodiments, the one or more SNPs are located at a gene comprising LRRK, NOD2, ATG16L1, SNF300, OLIG3, and/or PRKCB1. Non-limiting examples of SNPs located at the gene comprising LRRK, NOD2, ATG16L1, SNF300, OLIG3, and/or PRKCB1 LRRK, NOD2, ATG16L1, SNF300, OLIG3, and/or PRKCBlinclude SNPs from any one of Tables 2-4. In some embodiments, the genotype comprises one or more SNPs selected from Table 1. In some embodiments, the SNPs located at the genes described herein are associated with a likelihood that a subject is suitable for treatment of a disease or disorder with an antagonist of RIPK2 activity of expression, such as those described herein.

In some embodiments, the one or more SNPs is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰. In some embodiments, the one or more SNPs is associated with a risk that the subject has, or will develop, a subclinical phenotype (e.g., stricturing and/or penetrating disease) of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰.

In some embodiments, the genotype comprises one or more SNPs in linkage disequilibrium with rs5743289 as determined by an r² value of at least about 0.80, about 0.85, about 0.90, about 0.95, or about 1.0. In some instances, the r² value is 0.80. In some instances, the r² value is 0.85. In some instances, the r² value is 0.90. In some instances, the r² value is 0.95.

In some instances, the r² value is 1.0. In some embodiments, the one or more SNPs comprises rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” risk allele. In some embodiments, the SNP at rs2357623 comprises an “A” or a “G” risk allele. In some embodiments, the SNP at rs2066845 comprises a “G” or a “C” risk allele. In some embodiments, the SNP at rs5743289 comprises an “A” or a “G” risk allele. In some embodiments, the SNP at rs72796367 comprises a “G” or an “A” risk allele. In some embodiments, the SNP at rs6752107 comprises a “G” or an “A” risk allele. In some embodiments, the SNP at rs12994997 comprises a “G” or an “A” risk allele. In some embodiments, the SNP at rs11741861 comprises a “G” or an “A” In some embodiments, the SNP at rs9494844 comprises an “A” or a “C” risk allele. In some embodiments, the SNP at rs6918329 comprises a “G” or an “A” risk allele. In some embodiments, the SNP at rs7404095 comprises an “A” or a “G” risk allele. In some instances, the SNP is within a polynucleotide sequence provided in SEQ ID NOS: 1-11.

Methods of Treatment

Disclosed herein, in some embodiments, are methods of treating a disease or condition, or a symptom of the disease or condition, in a subject, comprising administrating of therapeutic effective amount of one or more therapeutic agents to the subject. In some embodiments, the one or more therapeutic agents are administered to the subject, provided a presence of a genotype described herein is detected in a sample obtained from the subject. In some embodiments, the one or more therapeutic agents are administered to the subject alone (e.g., standalone therapy). In some embodiments, the one or more therapeutic agents are administered in combination with an additional agent. In some embodiments, the therapeutic agent is a first-line therapy for the disease or condition. In some embodiments, the therapeutic agent is a second-line, third-line, or fourth-line therapy, for the disease or condition.

Therapeutic Agent

Disclosed herein, in some embodiments, are therapeutic agents useful for the treatment of a disease or condition, or symptom of the disease or condition, disclosed herein. Disclosed herein, in some embodiments, are modulators of RIPK2 activity or expression that are useful for the treatment of an inflammatory, fibrotic, and/or fibrostenotic disease or condition. In some embodiments, the inflammatory disease comprises inflammatory bowel disease (IBD), Crohn's disease (CD), and/or ulcerative colitis (UC). In some embodiments, a modulator of RIPK2 activity or expression comprises an antagonist or a partial antagonist of RIPK2. In some embodiments, the RIPK2 antagonist or partial antagonist comprises an antibody or antigen-binding fragment, or a small molecule.

In some embodiments, the RIPK2 antagonist or partial antagonist comprises a type I RIPK2 inhibitor effective to bind to the ATP binding pocket of an active conformation of the RIPK2 kinase domain. In some embodiments, the RIPK2 antagonist or partial antagonist comprises a type I½ RIPK2 inhibitor effective to bind to the ATP binding pocket of an inactive conformation of the RIPK2 kinase domain without displacing the RIPK2 kinase activation segment. In some embodiments, the RIPK2 antagonist or partial antagonist comprises a type II RIPK2 inhibitor effective to displace a RIPK2 kinase activation segment. In some embodiments, the RIPK2 antagonist or partial antagonist comprises a type III RIPK2 inhibitor effective to bind an allosteric site of RIPK2 located in the cleft between the small and large lobes adjacent to the ATP binding pocket. In some embodiments, the RIPK2 antagonist or partial antagonist comprises a type IV RIPK2 inhibitor effective to bind an allosteric site of RIPK2 located outside of the cleft and the phosphoacceptor region. In some embodiments, the RIPK2 antagonist or partial antagonist comprises a type V RIPK2 inhibitor effective to span two regions of the RIPK2 kinase domain. In some embodiments, the RIPK2 antagonist or partial antagonist comprises a type VI RIPK2 inhibitor effective to form a covalent adduct with RIPK2. In some embodiments, the RIPK2 antagonist or partial antagonist comprises a RIPK2 inhibitor effective to inhibit RIPK2 ubiquitination. In some embodiments, the RIPK2 antagonist or partial antagonist comprises a RIPK2 inhibitor effective to inhibit RIPK2 autophosphorylation. In some embodiments, the RIPK2 antagonist or partial antagonist comprises a RIPK2 inhibitor effective to block NOD-dependent tumor necrosis factor production without affecting lipopolysaccharide-dependent pathways. In some embodiments, the RIPK2 antagonist or partial antagonist comprises ponatinib, sorafenib, regorafenib, gefitinib, or erlotinib. In some embodiments, the RIPK2 antagonist or parital antagonist comprises GSK2983559, GSK583, Inhibitor 7, Biaryl Urea, CSR35, CSLP37, CSLP43, RIPK2 inhibitor 1, CS6, PP2, WEHI-345, SB203580, OD36, OD38, RIPK2-IN-8, RIPK2-IN-1, or RIPK2-IN-2, or any combination thereof.

Disclosed herein, in some embodiments, are antagonists or partial antagonists of RIPK2 having a structure of Formula (I) or a pharmaceutically acceptable salt or isotopic variant thereof:

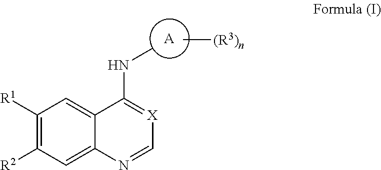

wherein

Ring A is C₃₋₈cycloalkyl, C₂₋₉heterocycloalkyl, C₂₋₉heteroaryl, or 6- to 10-membered aryl;
X is N or CR⁴;
R¹ and R² are independently —H, halogen, —OH, —OR⁵, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵;
each R³ is independently —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷;
R⁴ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl, wherein the alkyl, haloalkyl, cycloalkyl, phenyl, heteroaryl, and heterocycloalkyl are optionally substituted;
each R⁵ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ substituents are taken together with the nitrogen atom to which they are attached to form a 5- or 6-membered heterocycle; and
R⁷ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl; and n is 0, 1, 2, 3, 4, or 5.

In some embodiments of a compound of Formula (I), Ring A is C₃₋₈cycloalkyl, C₂₋₉-heterocycloalkyl, C₂₋₉heteroaryl, or 6- to 10-membered aryl. In some embodiments of a compound of Formula (I), Ring A is C₃₋₇heteroaryl or 6-membered aryl. In some embodiments of a compound of Formula (I), Ring A is pyrrazolyl. In some embodiments of a compound of Formula (I), Ring A is C₇heteroaryl. In some embodiments of a compound of Formula (I), Ring A is phenyl.

In some embodiments, for a compound of Formula (I), X is N or CR⁴. In some embodiments, for a compound of Formula (I), X is N or CH. In some embodiments, for a compound of Formula (I), X is N. In some embodiments, for a compound of Formula (I), X is CH.

In some embodiments, for a compound of Formula (I), R¹ is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (I), R¹ is C₁₋₆alkyl, C₂₋₆alkenyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (I), R¹ is —O—C₁₋₆alkyl, —O—C₁₋₆alkyl-OR⁵, —O—C₁₋₆alkyl-N(R⁶)₂, or —S(═O)₂R⁵. In some embodiments, for a compound of Formula (I), R¹ is —O—C₁₋₆alkyl. In some embodiments, for a compound of Formula (I), Ie is —OCH₃. In some embodiments, for a compound of Formula (I), R¹ is —O—C₁₋₆alkyl-OR⁵. In some embodiments, for a compound of Formula (I), R¹ is —OCH₂CH₂OCH₃. In some embodiments, for a compound of Formula (I), R¹ is —O—C₁₋₆alkyl- N(R⁶)₂. In some embodiments, for a compound of Formula (I), R¹ is —OCH₂CH₂CH₂morpholine. In some embodiments, for a compound of Formula (I), R¹ is —S(═O)₂R⁵. In some embodiments, for a compound of Formula (I), R¹ is —S(═O)₂tert-butyl.

In some embodiments, for a compound of Formula (I), R² is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (I), R² is —H, —O—C₁₋₆alkyl, —O—C₁₋₆alkyl-OR⁵, or —O—C₁₋₆alkyl-OH. In some embodiments, for a compound of Formula (I), R² is -H. In some embodiments, for a compound of Formula (I), R² is —O—C₁₋₆alkyl. In some embodiments, for a compound of Formula (I), R² is —OCH₃. In some embodiments, for a compound of Formula (I), R² is —O—C_{1- 6}alkyl—OR⁵. In some embodiments, for a compound of Formula (I), R² is —OCH₂CH₂OCH₃. In some embodiments, for a compound of Formula (I), R² is —O—C₁₋₆alkyl-OH. In some embodiments, for a compound of Formula (I), R² is —OCH₂CH₂OH.

In some embodiments, for a compound of Formula (I), R³ is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (I), R³ is —H, halogen, C₁₋₆alkyl, C₂₋₆alkynyl, or —O-phenyl. In some embodiments, for a compound of Formula (I), R³ is —H. In some embodiments, for a compound of Formula (I), R³ is —Cl. In some embodiments, for a compound of Formula (I), R³ is —F. In some embodiments, for a compound of Formula (I), R³ is —CH₃. In some embodiments, for a compound of Formula (I), R³ is —CCH. In some embodiments, for a compound of Formula (I), R³ is —O-phenyl.

In some embodiments, for a compound of Formula (I), n is 0, 1, 2, or 3. In some embodiments, for a compound of Formula (I), n is 1, 2, or 3. In some embodiments, for a compound of Formula (I), n is 1 or 2. In some embodiments, for a compound of Formula (I), n is 0. In some embodiments, for a compound of Formula (I), n is 1. In some embodiments, for a compound of Formula (I), n is 2. In some embodiments, for a compound of Formula (I), n is 3.

Disclosed herein, in some embodiments, are antagonists or partial antagonists of RIPK2 having a structure of Formula (Ia) or a pharmaceutically acceptable salt or isotopic variant thereof:

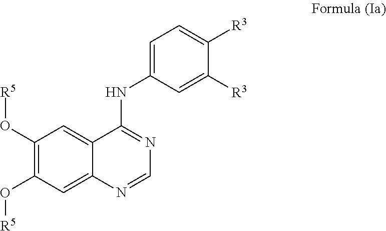

wherein;
each R³ is independently —H, halogen, —CCH, or —O-aryl; and
each R⁵ is independently C₁₋₆ alkyl, —C₁₋₆alkyl-O—C₁₋₆alkyl, or —C₁₋₆alkyl-heterocycloalkyl.

Disclosed herein, in some embodiments, are antagonists or partial antagonists of RIPK2 having a structure of Formula (Ia) or a pharmaceutically acceptable salt or isotopic variant thereof:

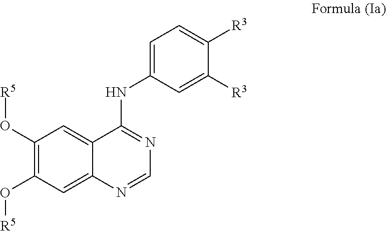

wherein;
each R³ is independently —H, —Cl, —F, —CCH, or —O-phenyl; and
each R⁵ is independently —CH₃, —CH₂CH₂OCH₃, or —CH₂CH₂CH₂ morpholine.

Disclosed herein, in some embodiments, are antagonists or partial antagonists of RIPK2 having a structure of Formula (Ib) or a pharmaceutically acceptable salt or isotopic variant thereof:

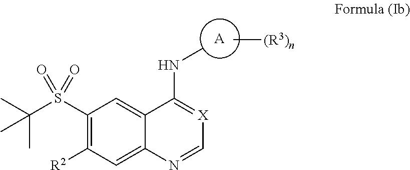

wherein;
Ring A is C₃₋₇heteroaryl;
X is N or CH;
R² is —H, —OC₁₋₆alkyl, or —O—C₁₋₆alkyl-OH;
each R³ is independently —H, —C₁₋₆alkyl, or halogen; and
n is 0, 1, or 2.

Disclosed herein, in some embodiments, are antagonists or partial antagonists of RIPK2 having a structure of Formula (Ib) or a pharmaceutically acceptable salt or isotopic variant thereof:

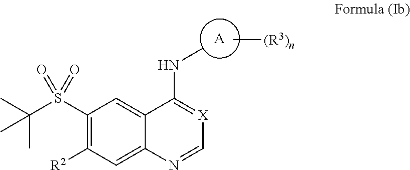

wherein;
Ring A is C₃₋₇heteroaryl;
X is N or CH;
R² is —H, —OCH₃, or —OCH₂CH₂OH;
each R³ is independently —H, —CH₃, or —F; and
n is 0, 1, or 2.

In some embodiments a compound of Formula (I) or a pharmaceutically acceptable salt or isotopic variant thereof has the structure of:

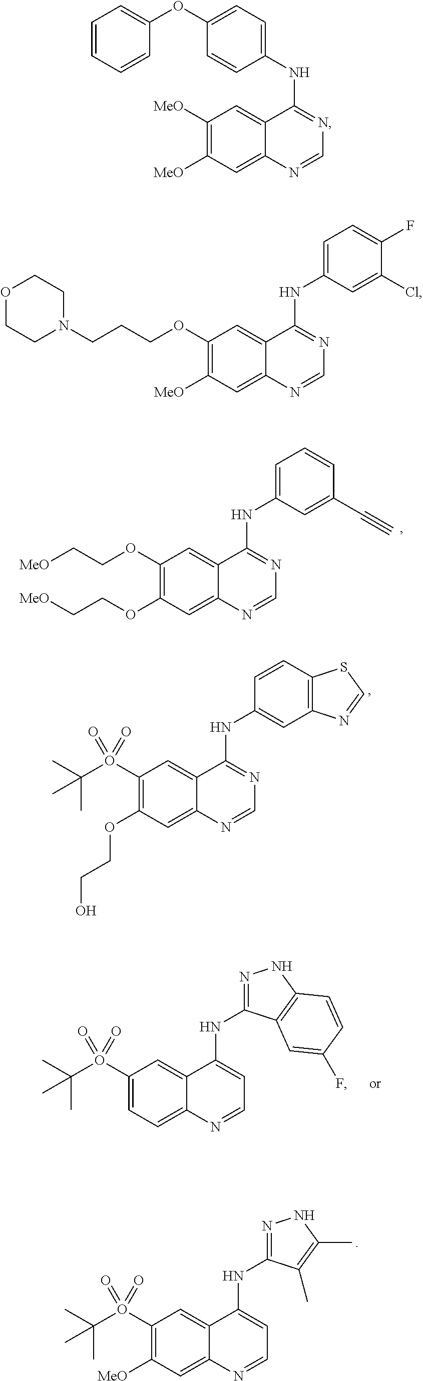

Disclosed herein, in some embodiments, are antagonists or partial antagonists of RIPK2 having a structure of Formula (II) or a pharmaceutically acceptable salt or isotopic variant thereof:

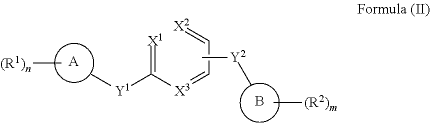

wherein
Rings A and B are independently C₃₋₈cycloalkyl, C₂₋₉heterocycloalkyl, C₂₋₉heteroaryl, or 6- to 10-membered aryl;
X¹, X², and X³ are independently N or CR⁴;
Y¹ and Y² are independently a bond, —O—, —S—, —C(R⁵)₂, —NR⁶—, —NR⁶C(O)—, —C(O)NR⁶—, or —NR⁶C(O)NR⁶—;
each R¹ and R² is independently —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —SCH₂C(O)OR⁵, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷;
each R⁴ is independently —H, halogen, —N(R⁶)₂, —NO₂, C₁₋₆alkyl, C₂₋₆alkenyl, C₂₋₆alkynyk C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl, wherein the alkyl, haloalkyl, cycloalkyl, phenyl, heteroaryl, and heterocycloalkyl are optionally substituted;
each R⁵ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ are taken together with the nitrogen atom to which they are attached to form a 5- or 6-membered heterocycle; and
R⁷ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl; and
m and n are each independently 0, 1, 2, 3, 4, or 5.

In some embodiments, for a compound of Formula (II), Rings A and B are independently C₃₋₈cycloalkyl, C₂₋₉heterocycloalkyl, C₂₋₉heteroaryl, or 6- to 10-membered aryl. In some embodiments, for a compound of Formula (II), Rings A and B are independently C₂₋₉heteroaryl or 6- to 10-membered aryl. In some embodiments, for a compound of Formula (II), Ring A is phenyl. In some embodiments, for a compound of Formula (II), Ring A is pyridyl. In some embodiments, for a compound of Formula (II), Ring A is furanyl. In some embodiments, for a compound of Formula (II), Ring B is phenyl. In some embodiments, for a compound of Formula (II), Ring B is pyrrazolyl. In some embodiments, for a compound of Formula (II), Ring B is pyridyl. In some embodiments, for a compound of Formula (II), Ring B is isoxazolyl. In some embodiments, for a compound of Formula (II), Ring A is phenyl and Ring B is pyrrazolyl. In some embodiments, for a compound of Formula (II), Ring A is phenyl and Ring B is phenyl. In some embodiments, for a compound of Formula (II), Ring A is phenyl and Ring B is pyridyl. In some embodiments, for a compound of Formula (II), Ring A is pyridyl and Ring B is phenyl. In some embodiments, for a compound of Formula (II), Ring A is pyridyl and Ring B is isoxazolyl. In some embodiments, for a compound of Formula (II), Ring A is isoxazoylyl and Ring B is pyridyl. In some embodiments, for a compound of Formula (II), Ring A is furanyl and Ring B is phenyl.

In some embodiments, for a compound of Formula (II), X¹, X², and X³ are independently N or CR⁴. In some embodiments, for a compound of Formula (II), X¹ is CH. In some embodiments, for a compound of Formula (II), X¹ is CF. In some embodiments, for a compound of Formula (II), X¹ is CCH₃. In some embodiments, for a compound of Formula (II), X¹ is CNH₂. In some embodiments, for a compound of Formula (II), X¹ is N. In some embodiments, for a compound of Formula (II), X² is CH. In some embodiments, for a compound of Formula (II), X² is CF. In some embodiments, for a compound of Formula (II), X² is N. In some embodiments, for a compound of Formula (II), X² is C—N-methylpyrazine. In some embodiments, for a compound of Formula (II), X³ is CH. In some embodiments, for a compound of Formula (II), X³ is N. In some embodiments, for a compound of Formula (II), X¹ is CF and X² and X³ are CH. In some embodiments, for a compound of Formula (II), X² is CF and X¹ and X³ are CH. In some embodiments, for a compound of Formula (II), X¹, X², and X³ are CH. In some embodiments, for a compound of Formula (II), X¹ is CCH₃ and X² and X³ are CH. In some embodiments, for a compound of Formula (II), X¹ is CNH₂, X² is N, and X³ is CH. In some embodiments, for a compound of Formula (II), X² is C—N-methylpyrazine and X¹ and X³ are N.

In some embodiments, for a compound of Formula (II), Y¹ and Y² are independently a bond, —O—, —S—, —C(R⁵₂, —NR⁶—, —NR⁶C(O)—, —C(O)NR⁶—, or —NR⁶C(O)NR⁶—. In some embodiments, for a compound of Formula (II), Y¹ is —NR⁶C(O)—. In some embodiments, for a compound of Formula (II), Y¹ is —O—. In some embodiments, for a compound of Formula (II), Y¹ is —NR⁶C(O)NR⁶—. In some embodiments, for a compound of Formula (II), Y¹ is a bond. In some embodiments, for a compound of Formula (II), Y¹ is —NR⁶—. In some embodiments, for a compound of Formula (II), Y² is —NR⁶C(O)—. In some embodiments, for a compound of Formula (II), Y² is —O—. In some embodiments, for a compound of Formula (II), Y² is —NR⁶C(O)NR⁶—. In some embodiments, for a compound of Formula (II), Y² is a bond. In some embodiments, for a compound of Formula (II), Y¹ is —S—. In some embodiments, for a compound of Formula (II), Y¹ and Y² are —NHC(O)—. In some embodiments, for a compound of Formula (II), Y¹ is —O— and Y² is —NHC(O)NH—. In some embodiments, for a compound of Formula (II), Y¹ is —NHC(O)NH— and Y² is —O—. In some embodiments, for a compound of Formula (II), Y¹ and Y² are bonds. In some embodiments, for a compound of Formula (II), Y¹ is —NH— and Y² is —S—.

In some embodiments, for a compound of Formula (II), R¹ is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (II), R¹ is —Cl. In some embodiments, for a compound of Formula (II), R¹ is —F. In some embodiments, for a compound of Formula (II), R¹ is —C(O)NHCH₃. In some embodiments, for a compound of Formula (II), R¹ is 2-methylpyrrazolyl. In some embodiments, for a compound of Formula (II), R¹ is N-methylimidazolyl. In some embodiments, for a compound of Formula (II), R¹ is tert-butyl. In some embodiments, for a compound of Formula (II), R¹ is —NHC(O)cyclopropyl. In some embodiments, for a compound of Formula (II), R¹ is —SCH₂C(O)OH. In some embodiments, for a compound of Formula (II), R¹ is —OCH₃. In some embodiments, for a compound of Formula (II), R¹ is —NHS(—O)₂CH₂CH₂CH₃.

In some embodiments, for a compound of Formula (II), R² is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (II), R² is —Cl. In some embodiments, for a compound of Formula (II), R² is —F. In some embodiments, for a compound of Formula (II), R² is —C(O)NHCH₃. In some embodiments, for a compound of Formula (II), R¹ is 2-methylpyrrazolyl. In some embodiments, for a compound of Formula (II), R¹ is N-methylimidazolyl. In some embodiments, for a compound of Formula (II), R² is —CH₂-(2-iso-propylimidazole). In some embodiments, for a compound of Formula (II), R² is tert-butyl. In some embodiments, for a compound of Formula (II), R² is —CH₃. In some embodiments, for a compound of Formula (II), R² is —C(O)NHCH3. In some embodiments, for a compound of Formula (II), R² is pyrazinyl.

In some embodiments, for a compound of Formula (II), m is 1 or 2. In some embodiments, for a compound of Formula (II), m is 1. In some embodiments, for a compound of Formula (II), m is 2. In some embodiments, for a compound of Formula (II), n is 1 or 2. In some embodiments, for a compound of Formula (II), n is 1. In some embodiments, for a compound of Formula (II), n is 2.

Disclosed herein, in some embodiments, are antagonists or partial antagonists of RIPK2 having a structure of Formula (IIa) or a pharmaceutically acceptable salt or isotopic variant thereof:

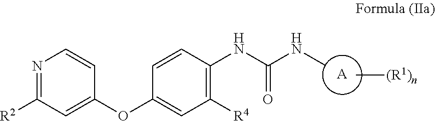

wherein

Ring A is phenyl or isoxazolyl;

each R¹ is independently C₁₋₆alkyl, halogen, —C₁₋₆fluoroalkyl, or —S—C₁₋₆alkyl-C(O)OH;

R² is —H or —C(O)NHCH₃;

R⁴ is —H or halogen; and

m is 1 or 2.

Disclosed herein, in some embodiments, are antagonists or partial antagonists of RIPK2 having a structure of Formula (IIa) or a pharmaceutically acceptable salt or isotopic variant thereof:

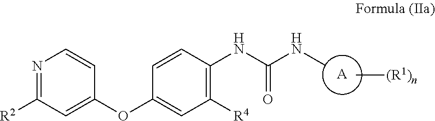

wherein

Ring A is phenyl or isoxazolyl;

each R¹ is independently tert-butyl, —Cl, —F, —CF₃, or —SCH₂C(O)OH;

R² is —H or —C(O)NHCH₃;

R⁴ is —H or halogen; and

m is 1 or 2.

Disclosed herein, in some embodiments, are antagonists or partial antagonists of RIPK2 having a structure of Formula (IIb) or a pharmaceutically acceptable salt or isotopic variant thereof:

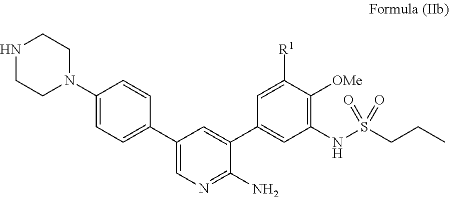

wherein

R¹ is halogen or —OR⁵.

In some embodiments a compound of Formula (II) or a pharmaceutically acceptable salt or isotopic variant thereof has the structure of:

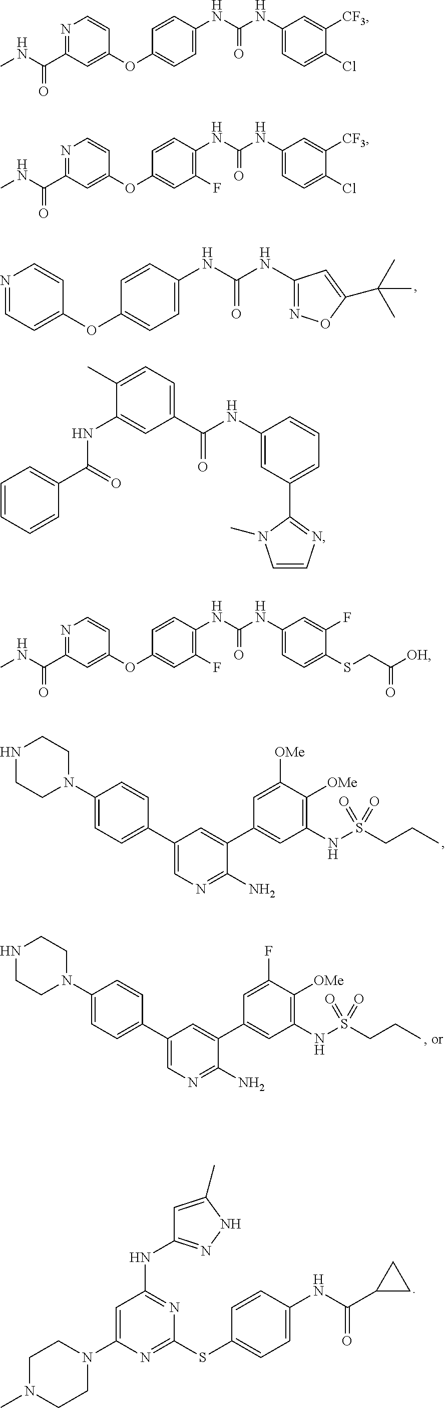

Disclosed herein, in some embodiments, are antagonists or partial antagonists of RIPK2 having a structure of Formula (III) or a pharmaceutically acceptable salt or isotopic variant thereof:

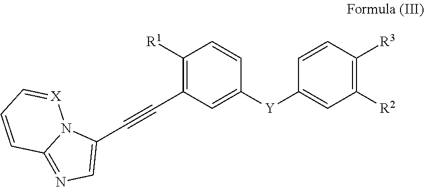

wherein
X is N or CR⁴;
Y is a bond, —O—, —S—, —C(R⁵₂, —NR⁶C(O)—, —C(O)NR⁶-, or —NR⁶C(O)NR⁶—;
R¹ is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-N(R⁶)₂, —O—C₁₋₆alkyl-N(R⁶)₂, or —S(═O)₂R⁵;
R² and R³ are independently —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷; or
   R² and R³ are taken together with the atoms to which they are attached to form an optionally substituted C₃₋₈cycloalkyl; and
R⁴ is hydrogen, halogen, —N(R⁶)₂, —NO₂, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl, wherein the alkyl, haloalkyl, cycloalkyl, phenyl, heteroaryl, and heterocycloalkyl are optionally substituted;
R⁵ is —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ substituents are taken together with the nitrogen atom to which they are attached to form a 5- or 6-membered heterocycle; and
R⁷ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl.

In some embodiments, for a compound of Formula (III), X is N or CR⁴. In some embodiments, for a compound of Formula (III), X is N and CH. In some embodiments, for a compound of Formula (III), X is N. In some embodiments, for a compound of Formula (III), X is CH.

In some embodiments, for a compound of Formula (III), Y is a bond, —O—, —S—, —C(R⁵)₂, —NR₆—, —NR₆C(O)—, —C(O)NR⁶—, or —NR⁶C(O)NR⁶—. In some embodiments, for a compound of Formula (III), Y is —NR⁶C(O)— or —C(O)NR⁶—. In some embodiments, for a compound of Formula (III), Y is —NHC(O)—. In some embodiments, for a compound of Formula (III), Y is —C(O)NH—.

In some embodiments, for a compound of Formula (III), R¹ is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-N(R⁶)₂, —O—C₁₋₆alkyl, —O—C₁₋₆alkyl-OH, —O—C₁₋₆alkyl-N(R⁶)₂, or —S(═O)₂R⁵. In some embodiments, for a compound of Formula (III), R¹ is —H, halogen, —OH, —CN, —N(R⁶)₂, C₁₋₆alkyl, C₂₋₆alkynyl, or C₃₋₈cycloalkyl. In some embodiments, for a compound of Formula (III), R¹ is C₁₋₆alkyl. In some embodiments, for a compound of Formula (III), R¹ is —CH₃. In some embodiments, for a compound of Formula (III), R¹ is tert-butyl.

In some embodiments, for a compound of Formula (III), R² is —H, halogen, —NO₂, —CN—, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷, or R² and R³ are taken together with the atoms to which they are attached to form an optionally substituted C₃₋₈cycloalkyl. In some embodiments, for a compound of Formula (III), R² is —H, halogen, —NO₂, —CN, —OH, —OR⁵, C₁₋₆alkyl, C₁₋₆haloalkyl, C₁₋₆heteroalkyl, C₃₋₈cycloalkyl, C₂₋₉heterocycloalkyl, C₂₋₉heteroaryl, or 6- to 10-membered aryl, or R² and R³ are taken together with the atoms to which they are attached to form an optionally substituted C₃₋₈cycloalkyl. In some embodiments, for a compound of Formula (III), R² is C₁₋₆alkyl, C₁₋₆haloalkyl, or C₃₋₈cycloalkyl, or R² and R³ are taken together with the atoms to which they are attached to form an optionally substituted C₃₋₈cycloalkyl. In some embodiments, for a compound of Formula (III), R² is —CH₃, —CF₃, or cyclopropyl, or R² and R³ are taken together with the atoms to which they are attached to form an optionally substituted C₃₋₈cycloalkyl. In some embodiments, for a compound of Formula (III), R² is —CH₃, —CF₃, or cyclopropyl. In some embodiments, for a compound of Formula (III), R² is —CH₃. In some embodiments, for a compound of Formula (III), R² is —CF₃. In some embodiments, for a compound of Formula (III), R² is cyclopropyl. In some embodiments, for a compound of Formula (III), R² and R³ are taken together with the atoms to which they are attached to form a C₅ cycloalkyl. In some embodiments, for a compound of Formula (III), R² and R³ are taken together with the atoms to which they are attached to form a C₅ cycloalkyl substituted with an N-methylpiperazine.

In some embodiments, for a compound of Formula (III), R³ is —H, halogen, —NO₂, —CN, —OH, —OR⁵, —SR⁵, —N(R⁶)₂, N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷, or R² and R³ are taken together with the atoms to which they are attached to form an optionally substituted C₃₋₈cycloalkyl. In some embodiments, for a compound of Formula (III), R³ is —H, halogen, —CN, —OR⁵, —N(R⁶)₂, —S(═O)₂R⁵, —C(O)R⁵, —C(O)OR⁵, —NR⁶C(O)N(R⁶)₂, —NR⁶C(O)R⁵, —NR⁶C(O)OR⁵, C₁₋₆alkyl, C₁₋₆heteroalkyl, C₂₋₉heterocycloalkyl, or C₂₋₉heteroaryl, wherein each alkyl, heteroalkyl, heterocycloalkyl, and heteroaryl is optionally substituted with one or more R⁷, or R² and R³ are taken together with the atoms to which they are attached to form an optionally substituted C₃₋₈cycloalkyl. In some embodiments, for a compound of Formula (III), R³ is C₁₋₆alkyl substituted with C₂₋₉heterocycloalkyl. In some embodiments, for a compound of Formula (III), R³ is CH₂—N-methylpiperazine. In some embodiments, for a compound of Formula (III), R² and R³ are taken together with the atoms to which they are attached to form a C₅ cycloalkyl. In some embodiments, for a compound of Formula (III), R² and R³ are taken together with the atoms to which they are attached to form a C₅ cycloalkyl substituted with an N-methylpiperazine.

Disclosed herein, in some embodiments, are antagonists or partial antagonists of RIPK2 having a structure of Formula (III) or a pharmaceutically acceptable salt or isotopic variant thereof:

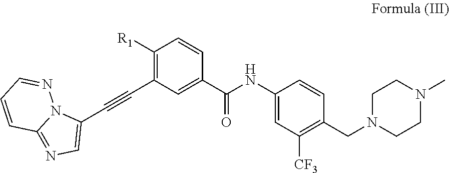

wherein

R¹ is C₁₋₆alkyl.

In some embodiments a compound of Formula (III) or a pharmaceutically acceptable salt or isotopic variant thereof has the structure of:

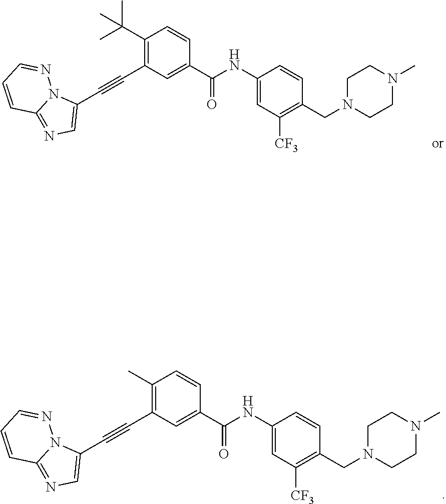 [SMILES: CN1CCN(Cc2ccc(NC(=O)c3ccc(C(C)(C)C)c(C#Cc4cnc5cccnn45)c3)cc2C(F)(F)F)CC1 ; Cc1ccc(C(=O)Nc2ccc(CN3CCN(C)CC3)c(C(F)(F)F)c2)cc1C#Cc1cnc2cccnn12]

Disclosed herein, in some embodiments, are antagonists or partial antagonists of RIPK2 having a structure of Formula (IV) or a pharmaceutically acceptable salt or isotopic variant thereof:

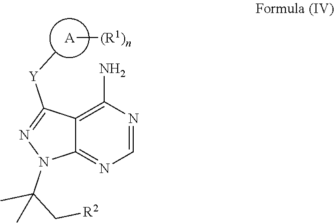

wherein
Ring A is C₃₋₈cycloalkyl, C₂₋₉heterocycloalkyl, C₂₋₉heteroaryl, or 6- to 10-membered aryl;
Y is a bond, —O—, —S—, —C(R⁵)₂—, —NR⁶—, —NR⁶C(O)—, —C(O)NR⁶—, or —NR⁶C(O)NR⁶—;
R¹ is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵;
R² is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶₂, —NR⁶C(O)R⁵, —NR⁶C(O)OR⁵, C₁₋₆alkyl, C_{2- 6}alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷;
R⁵ is —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ substituents are taken together with the nitrogen atom to which they are attached to form a 5- or 6-membered heterocycle;
R⁷ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl; and
n is 0, 1, 2, 3, 4, or 5.

In some embodiments, for a compound of Formula (IV), Ring A is C₃₋₈cycloalkyl, C₂₋₉heterocycloalkyl, C₂₋₉heteroaryl, or 6- to 10-membered aryl. In some embodiments, for a compound of Formula (IV), Ring A is 6- to 10-membered aryl. In some embodiments, for a compound of Formula (IV), Ring A is phenyl. In some embodiments, for a compound of Formula (IV), Ring A is naphthyl.

In some embodiments, for a compound of Formula (IV), Y is a bond, —O—, —S—, —C(R⁵)₂—, —NR⁶—, —NR⁶C(O)—, —C(O)NR⁶—, or —NR⁶C(O)NR⁶—. In some embodiments, for a compound of Formula (IV), Y is a bond or —C(R⁵)₂—. In some embodiments, for a compound of Formula (IV), Y is a bond. In some embodiments, for a compound of Formula (IV), Y is —CH₂—.

In some embodiments, for a compound of Formula (IV), R¹ is —H, halogen, —OH, —CN, —N(R⁶₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (IV), R¹ is —H, halogen, or C₁₋₆alkyl. In some embodiments, for a compound of Formula (IV), R¹ is —H, —Cl, or CH₃. In some embodiments, for a compound of Formula (IV), R¹ is —H. In some embodiments, for a compound of Formula (IV), R¹ is —Cl. In some embodiments, for a compound of Formula (IV), R¹ is —CH₃.

In some embodiments, for a compound of Formula (IV), R² is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (IV), R² is —H or —NR⁶C(O)R⁵. In some embodiments, for a compound of Formula (IV), R² is —H or —NR⁶C(O)C₂₋₉heteroaryl. In some embodiments, for a compound of Formula (IV), R² is —H. In some embodiments, for a compound of Formula (IV), R² is —NHC(O)pyridyl.

In some embodiments, for a compound of Formula (IV), n is 1, 2, or 3. In some embodiments, for a compound of Formula (IV), n is 1 or 2. In some embodiments, for a compound of Formula (IV), n is 1. In some embodiments, for a compound of Formula (IV), n is 2. In some embodiments, for a compound of Formula (IV), n is 3.

Disclosed herein, in some embodiments, are antagonists or partial antagonists of RIPK2 having a structure of Formula (IVa) or a pharmaceutically acceptable salt or isotopic variant thereof:

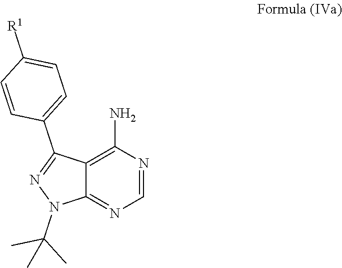

wherein

R¹ is halogen or C₁₋₆alkyl.

Disclosed herein, in some embodiments, are antagonists or partial antagonists of RIPK2 having a structure of Formula (IVb) or a pharmaceutically acceptable salt or isotopic variant thereof:

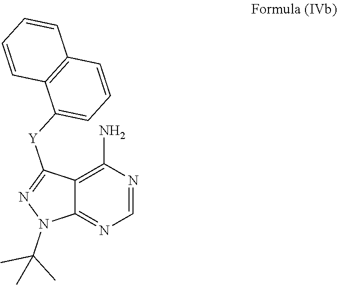

wherein Y is a bond or —C₁₋₃alkyl-.

Disclosed herein, in some embodiments, are antagonists or partial antagonists of RIPK2 having a structure of Formula (IVb) or a pharmaceutically acceptable salt or isotopic variant thereof:

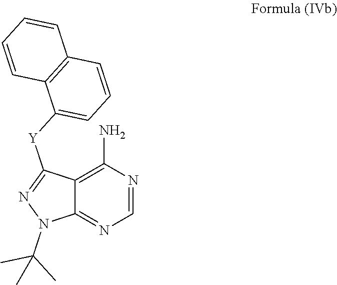

wherein

Y is a bond or —CH₂—.

In some embodiments a compound of Formula (IV) or a pharmaceutically acceptable salt or isotopic variant thereof has the structure of:

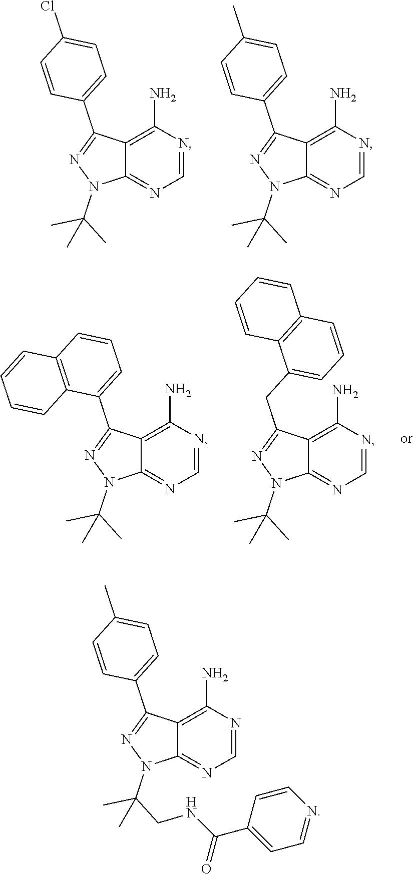

Disclosed herein, in some embodiments, are antagonists or partial antagonists of RIPK2 having a structure of Formula (V) or a pharmaceutically acceptable salt or isotopic variant thereof:

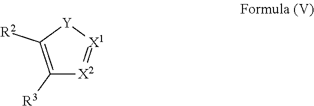

wherein
X¹ and X² are independently N or CR⁴;
Y is S, O, or NR¹;
R¹ is —H, —S(═O)₂R⁵, —S(═O)₂N(R⁶)₂, —C(O)R⁵, —C(O)OR⁵, —C(O)N(R⁶)₂, C₁₋₆alkyl, C₂₋₆alkenyl, C₂₋₆alkynyl, C₁₋₆haloalkyl, C₁₋₆heteroalkyl, C₃₋₈cycloalkyl, C₂₋₉heterocycloalkyl, C₂₋₉heteroaryl, or 6- to 10-membered aryl, wherein each alkyl, haloalkyl, heteroalkyl, cycloalkyl, heterocycloalkyl, aryl, and heteroaryl is optionally substituted with one or more R⁷;
R² is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷; or
   R¹ and R² are taken together with the atoms to which they are attached to form an optionally substituted C₃₋₈heterocycloalkyl; and
R³ and R⁴ are independently —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)2N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷;
R⁵ is —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈ cycloalkyl, —C₁₋₆alkyl-C₃₋₈cycloalkyl, phenyl, —C₁₋₆alkyl-phenyl, or C₂₋₉heteroaryl; or
   two R⁶ substituents are taken together with the nitrogen atom to which they are attached to form a 5- or 6-membered heterocycle; and
R⁷ is —H, halogen, —S(═O)CH₃, —N(R⁶)₂, —C(O)N(R⁶)₂, C₁₋₆alkyl, C₂₋₆alkenyl, C₂₋₆alkynyl, C₁₋₆haloalkyl, C₁₋₆heteroalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl.

In some embodiments, for a compound of Formula (V), X¹ and X² are independently N or CR⁴. In some embodiments, for a compound of Formula (V), X¹ is N. In some embodiments, for a compound of Formula (V), X¹ is CR⁴. In some embodiments, for a compound of Formula (V), X² is N. In some embodiments, for a compound of Formula (V), X² is CR⁴. In some embodiments, for a compound of Formula (V), X¹ is N and X² is CR⁴. In some embodiments, for a compound of Formula (V), X¹ is CR⁴ and X² is N.

In some embodiments, for a compound of Formula (V), Y is S, O, or NR¹. In some embodiments, for a compound of Formula (V), Y is S. In some embodiments, for a compound of Formula (V), Y is NH. In some embodiments, for a compound of Formula (V), Y is NR¹.

In some embodiments, for a compound of Formula (V), R¹ is —H, —S(═O)₂R⁵, —S(═O)₂N(R⁶)₂, —C(O)R⁵, —C(O)OR⁵, —C(O)N(R⁶)₂, C₁₋₆alkyl, C₂₋₆alkenyl, C₂₋₆alkynyl, C₁₋₆haloalkyl, C₁₋₆heteroalkyl, C₃₋₈cycloalkyl, C₂₋₉heterocycloalkyl, C₂₋₉heteroaryl, or 6- to 10-membered aryl, wherein each alkyl, haloalkyl, heteroalkyl, cycloalkyl, heterocycloalkyl, aryl, and heteroaryl is optionally substituted with one or more R⁷. In some embodiments, for a compound of Formula (V), R¹ is —H, C₁₋₆alkyl, C₃₋₈cycloalkyl, C₂₋₉heterocycloalkyl, C₂₋₉heteroaryl, or 6- to 10-membered aryl, wherein each cycloalkyl, heterocycloalkyl, aryl, and heteroaryl is optionally substituted with one or more R⁷. In some embodiments, for a compound of Formula (V), R¹ is aryl optionally substituted with one or more R⁷. In some embodiments, for a compound of Formula (V), R¹ is 2,4-dichlorophenyl.

In some embodiments, for a compound of Formula (V), R² is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷, or R¹ and R² are taken together with the atoms to which they are attached to form an optionally substituted C₃₋₈heterocycloalkyl. In some embodiments, for a compound of Formula (V), R² is —H, halogen, —S(═O)₂R⁵, —NR⁶S(═O)₂R⁵, —S(═O)₂N(R⁶)₂, —C(O)R⁵, —C(O)OR⁵, —C(O)N(R⁶)₂, C₁₋₆alkyl, C₂₋₆alkenyl, C₂₋₆alkynyl, C₃₋₈cycloalkyl, C₂₋₉heterocycloalkyl, C₂₋₉heteroaryl, 6- to 10-membered aryl, or —O— phenyl, wherein each alkyl, cycloalkyl, heterocycloalkyl, aryl, and heteroaryl is optionally substituted with one or more R⁷, or R¹ and R² are taken together with the atoms to which they are attached to form an optionally substituted C₃₋₈heterocycloalkyl. In some embodiments, for a compound of Formula (V), R² is —C(O)N(R⁶)₂ or 6-membered aryl optionally substituted with one or more R⁷. In some embodiments, for a compound of Formula (V), R² is 4-fluorophenyl. In some embodiments, for a compound of Formula (V), R² is 4-chlorophenyl. In some embodiments, for a compound of Formula (V), R² is 2-methylpyridinyl. In some embodiments, for a compound of Formula (V), R² is —C(O)NH—(2-methyl-6-chlorophenyl). In some embodiments, for a compound of Formula (V), R¹ and R² are taken together with the atoms to which they are attached to form an optionally substituted C₃₋₈heterocycloalkyl. In some embodiments, for a compound of Formula (V), R¹ and R² are taken together with the atoms to which they are attached to form a C₅ heterocycloalkyl.

In some embodiments, for a compound of Formula (V), R³ is —H, halogen, —NO₂, —CN, —OH, —OR⁵, —SR⁵, —N(R⁶)₂, N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (V), R³ is —H, C₁₋₆alkyl, C₂₋₆alkenyl, C₂₋₆alkynyl, C₁₋₆haloalkyl, C₁₋₆heteroalkyl, C3-8cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (V), R³ is —H or C₂₋₉heteroaryl optionally substituted with one or more R⁷. In some embodiments, for a compound of Formula (V), R³ is H. In some embodiments, for a compound of Formula (V), R³ is C₂₋₉heteroaryl optionally substituted with one or more R⁷. In some embodiments, for a compound of Formula (V), R³ is optionally substituted pyridinyl. In some embodiments, for a compound of Formula (V), R³ is optionally substituted quinolinyl. In some embodiments, for a compound of Formula (V), R³ is optionally substituted [1,2,4]triazolopyridinyl.

In some embodiments, for a compound of Formula (V), R⁴ is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (V), R⁴ is —H, —N(R⁶)₂, —C(O)R⁵, —C(O)OR⁵, —OC(O)R⁵, —C(O)N(R⁶)₂, —OC(O)N(R⁶)₂, C₁₋₆alkyl, C₂₋₆alkenyl, C₂₋₆alkynyl, C₁₋₆haloalkyl, C₁₋₆heteroalkyl, C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (V), R⁴ is —N(R⁶)₂, C₁₋₆alkyl, C₂₋₉heteroaryl, or 6- to 10-membered aryl, wherein each aryl and heteroaryl is optionally substituted with one or more R⁷. In some embodiments, for a compound of Formula (V), R⁴ is optionally substituted phenyl. In some embodiments, for a compound of Formula (V), R⁴ is optionally substituted pyridyl. In some embodiments, for a compound of Formula (V), R⁴ is —NH pyrimidine. In some embodiments, for a compound of Formula (V), R⁴ is —CH₂phenyl. In some embodiments, for a compound of Formula (V), R⁴ is CH2NHphenyl.

In some embodiments a compound of Formula (V) or a pharmaceutically acceptable salt or isotopic variant thereof has the structure of:

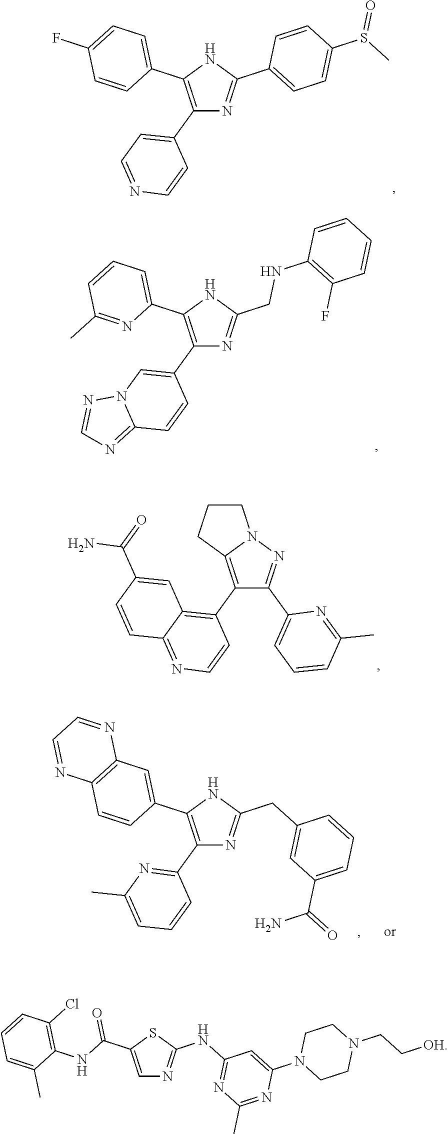

Disclosed herein, in some embodiments, are antagonists or partial antagonists of RIPK2 having a structure of Formula (VI) or a pharmaceutically acceptable salt or isotopic variant thereof:

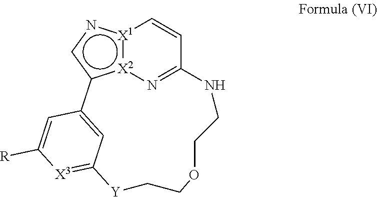

wherein
X¹ and X² are independently N or C;
X³ is N or CR⁴;
Y is a bond, —O—, —S—, —C(R⁵₂, —NR⁶—, —NR⁶C(O)—, —C(O)NR⁶—, or —NR⁶C(O)NR⁶—;
R is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷;
R⁴ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl, wherein the alkyl, haloalkyl, cycloalkyl, phenyl, heteroaryl, and heterocycloalkyl are optionally substituted;
R⁵ is —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ are taken together with the nitrogen atom to which they are attached to form a 5- or 6-membered heterocycle; and
R⁷ is —H, halogen, —S(═O)CH₃, —N(R⁶)₂, —C(O)N(R⁶)₂, C₁₋₆alkyl, C₂₋₆alkenyl, C₂₋₆alkynyl, C₁₋₆haloalkyl, C₁₋₆heteroalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or -C₁₋₆alkyl-C₂₋₉heteroaryl.

In some embodiments, for a compound of Formula (VII), X¹ and X² are independently N or C. In some embodiments, for a compound of Formula (VII), X¹ is N. In some embodiments, for a compound of Formula (VII), X¹ is C. In some embodiments, for a compound of Formula (VII), X² is N. In some embodiments, for a compound of Formula (VII), X² is C. In some embodiments, for a compound of Formula (VII), X¹ is N and X² is C. In some embodiments, for a compound of Formula (VII), X¹ is C and X² is N.

In some embodiments, for a compound of Formula (VII), X³ is N or CR⁴. In some embodiments, for a compound of Formula (VII), X³ is N or CH. In some embodiments, for a compound of Formula (VII), X³ is N. In some embodiments, for a compound of Formula (VII), X³ is CH.

In some embodiments, for a compound of Formula (VI), Y is a bond, —O—, —S—, —C(R⁵)₂, —NR⁶—, —NR⁶C(O)—, —C(O)NR⁶—, or —NR⁶C(O)NR⁶—. In some embodiments, for a compound of Formula (VI), Y is —O— or —NR⁶—. In some embodiments, for a compound of Formula (VI), Y is —O— or —NH—. In some embodiments, for a compound of Formula (VI), Y is —O—. In some embodiments, for a compound of Formula (VI), Y is —NH—.

In some embodiments, for a compound of Formula (VI), R is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶)₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (VI), R is —H, halogen, —S(═O)₂R⁵, —NR⁶S(═O)₂R⁵, —S(═O)2N(R⁶)₂, —C(O)R⁵, —C(O)OR⁵, —OC(O)R⁵, —C(O)N(R⁶)₂, —OC(O)N(R⁶)₂, C₁₋₆alkyl, C₂₋₆alkenyl, C₁₋₆heteroalkyl, C₃₋₈cycloalkyl, C₂₋₉heterocycloalkyl, C₂₋₉heteroaryl, 6- to 10-membered aryl, or —O-phenyl, wherein each alkyl, heteroalkyl, cycloalkyl, heterocycloalkyl, aryl, and heteroaryl is optionally substituted with one or more R⁷. In some embodiments, for a compound of Formula (VI), R is —H or halogen. In some embodiments, for a compound of Formula (VI), R is —H. In some embodiments, for a compound of Formula (VI), R is —Cl.

In some embodiments a compound of Formula (VI) or a pharmaceutically acceptable salt or isotopic variant thereof has the structure of:

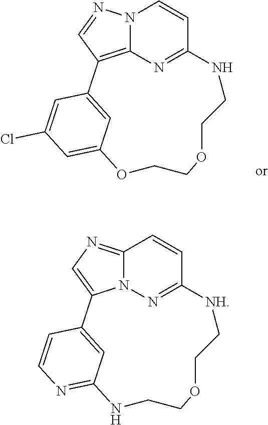 [SMILES: Clc1cc2cc(c1)-c1cnn3ccc(nc13)NCCOCCO2 ; c1cc2cc(n1)NCCOCCNc1ccc3ncc-2n3n1]

Disclosed herein, in some embodiments, are antagonists or partial antagonists of RIPK2 having a structure of Formula (VII) or a pharmaceutically acceptable salt or isotopic variant thereof:

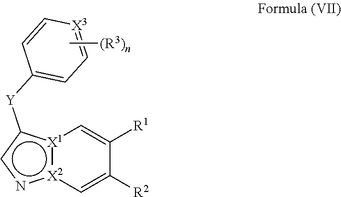

wherein
X¹ and X² are independently N or C;
X³ is N or CR⁴;
Y is a bond, —O—, —S—, —C(R⁵₂, —NR⁶—, —NR⁶C(O)—, —C(O)NR⁶—, or —NR⁶C(O)NR⁶—;
R¹ and R² are independently —H, halogen, —OH, —CN, —N(R⁶₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵;
R³ is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶)₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or —O-phenyl, wherein each alkyl, haloalkyl, heteroalkyl, cycloalkyl, heterocycloalkyl, aryl, and heteroaryl is optionally substituted with one or more R⁷;
R⁴ is —H, halogen, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl, wherein the alkyl, haloalkyl, cycloalkyl, phenyl, heteroaryl, and heterocycloalkyl are optionally substituted;
R⁵ is —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl;
each R⁶ is independently —H, C₁₋₆alkyl, C₂₋₆alkenyl, C₂₋₆alkynyl, C₁₋₆haloalkyl, C₃₋₈cycloalkyl, —C₁₋₆alkyl-C₃₋₈cycloalkyl, phenyl, —C₁₋₆alkyl-phenyl, or C₂₋₉heteroaryl; or
   two R⁶ substituents are taken together with the nitrogen atom to which they are attached to form a 5- or 6-membered heterocycle;
R⁷ is —H, halogen, —S(═O)CH₃, —N(R⁶)₂, —C(O)N(R⁶)₂, C₁₋₆alkyl, C₂₋₆alkenyl, C₂₋₆alkynyl, C₁₋₆haloalkyl, C₁₋₆heteroalkyl, C₃₋₈cycloalkyl, —C₁₋₆alkyl-C₃₋₈cycloalkyl, phenyl, —C₁₋₆alkyl-phenyl, C₂₋₉heterocycloalkyl, —C₁₋₆alkyl-C₂₋₉heterocycloalkyl, C₂₋₉heteroaryl, or —C₁₋₆alkyl-C₂₋₉heteroaryl; and
n is 0, 1, 2, 3, 4, or 5.

In some embodiments, for a compound of Formula (VII), X¹ and X² are independently N or C. In some embodiments, for a compound of Formula (VII), X¹ is N. In some embodiments, for a compound of Formula (VII), X¹ is C. In some embodiments, for a compound of Formula (VII), X² is N. In some embodiments, for a compound of Formula (VII), X² is C. In some embodiments, for a compound of Formula (VII), X¹ is N and X² is C. In some embodiments, for a compound of Formula (VII), X¹ is C and X² is N.

In some embodiments, for a compound of Formula (VII), X³ is N or CR⁴. In some embodiments, for a compound of Formula (VII), X³ is N or CH. In some embodiments, for a compound of Formula (VII), X³ is N. In some embodiments, for a compound of Formula (VII), X³ is CH.

In some embodiments, for a compound of Formula (VII), Y is a bond, —O—, —S—, —C(R⁵)₂, —NR⁶—, —NR⁶C(O)—, —C(O)NR⁶—, or —NR⁶C(O)NR⁶—. In some embodiments, for a compound of Formula (VII), Y is a bond, —NR⁶C(O)—, or —C(O)NR⁶—. In some embodiments, for a compound of Formula (VII), Y is a bond, —NHC(O)—, or —C(O)NH—. In some embodiments, for a compound of Formula (VII), Y is a bond. In some embodiments, for a compound of Formula (VII), Y is —NHC(O)—. In some embodiments, for a compound of Formula (VII), Y is —C(O)NH—.

In some embodiments, for a compound of Formula (VII), R¹ is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (VII), R¹ is —H, halogen, —N(R⁶₂, —NR⁶C(O)R⁵, C₁₋₆alkyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl-N(R⁶)₂, or —S(═O)₂R⁵. In some embodiments, for a compound of Formula (VII), R¹ is —H or —S(═O)₂R⁵. In some embodiments, for a compound of Formula (VII), R¹ is —H. In some embodiments, for a compound of Formula (VII), R¹ is —S(═O)₂iso-propyl. In some embodiments, for a compound of Formula (VII), R¹ is —S(═O)₂tert-butyl.

In some embodiments, for a compound of Formula (VII), R² is —H, halogen, —OH, —CN, —N(R⁶)₂, —NR⁶C(O)R⁵, —C(O)OR⁵, —C(O)N(R⁶)₂, C₁₋₆alkyl, C₂₋₆alkenyl, C₂₋₆alkynyl, C₃₋₈cycloalkyl, —C₁₋₆alkyl-OH, —C₁₋₆alkyl-OR⁵, —C₁₋₆alkyl-N(R⁶)₂, —O—C₁₋₆alkyl, —O—C₁₋₆alkyl-OH, —O—C₁₋₆alkyl-OR⁵, —O—C₁₋₆alkyl-N(R⁶)₂, or —S(═O)₂R⁵. In some embodiments, for a compound of Formula (VII), R² is —H, C₁₋₆alkyl, C₂₋₆alkenyl, C₂₋₆alkynyl, C₃₋₈cycloalkyl, —O—C₁₋₆alkyl, —O—C₁₋₆alkyl-OH, or —O—C₁₋₆alkyl-OR⁵. In some embodiments, for a compound of Formula (VII), R² is —H or —O—C₁₋₆alkyl. In some embodiments, for a compound of Formula (VII), R² is —H. In some embodiments, for a compound of Formula (VII), R² is —OCH₃. In some embodiments, for a compound of Formula (VII), R² is —OCH₂CH₃.

In some embodiments, for a compound of Formula (VII), R³ is —H, halogen, —NO₂, —CN, —OH, —OR⁵, —SR⁵, —N(R⁶)₂, —S(O)R⁵, —S(═O)₂R⁵, —NR⁶S(═O)₂R⁵, —S(═O)₂N(R⁶)₂, —C(O)R⁵, —C(O)OR⁵, —OC(O)R⁵, —C(O)N(R⁶)₂, —OC(O)N(R⁶₂, —NR⁶C(O)N(R⁶₂, —NR⁶C(O)R⁵, —NR⁶C(O)OR⁵, C₁₋₆alkyl, C₂₋₆alkenyl, C₂₋₆alkynyl, C₁₋₆haloalkyl, C₁₋₆heteroalkyl, —O—C₁₋₆alkyl, C₃₋₈cycloalkyl, C₂₋₉heterocycloalkyl, C₂₋₉heteroaryl, 6- to 10-membered aryl, or 13 O— phenyl, wherein each alkyl, haloalkyl, heteroalkyl, cycloalkyl, heterocycloalkyl, aryl, and heteroaryl is optionally substituted with one or more R⁷. In some embodiments, for a compound of Formula (VII), R³ is —H, halogen, —N(R⁶)₂, —S(═O)₂R⁵, —NR⁶S(═O)₂R⁵, —S(═O)₂N(R⁶)₂, —NR⁶C(O)N(R⁶)₂, —NR⁶C(O)R⁵, —NR⁶C(O)OR⁵, C₁₋₆alkyl, C₁₋₆heteroalkyl, —O—C₁₋₆alkyl, C₃₋₈cycloalkyl, C₂₋₉heterocycloalkyl, C₂₋₉heteroaryl, or 6- to 10-membered aryl, wherein each alkyl, heteroalkyl, cycloalkyl, heterocycloalkyl, aryl, and heteroaryl is optionally substituted with one or more R⁷. In some embodiments, for a compound of Formula (VII), R³ is —H, halogen, —N(R⁶)₂, or C₁₋₆alkyl. In some embodiments, for a compound of Formula (VII), R³ is —H. In some embodiments, for a compound of Formula (VII), R³ is —Cl. In some embodiments, for a compound of Formula (VII), R³ is —F. In some embodiments, for a compound of Formula (VII), R³ is —CH₃.

In some embodiments a compound of Formula (VII) or a pharmaceutically acceptable salt or isotopic variant thereof has the structure of:

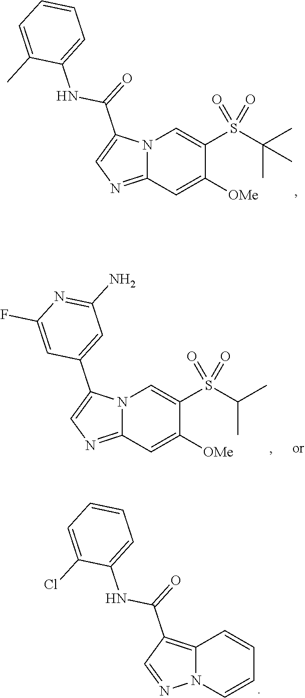 [SMILES: COc1cc2ncc(C(=O)Nc3ccccc3C)n2cc1S(=O)(=O)C(C)(C)C ; COc1cc2ncc(-c3cc(N)nc(F)c3)n2cc1S(=O)(=O)C(C)C ; O=C(Nc1ccccc1Cl)c1cnn2ccccc12]

Disclosed herein, in some embodiments, are antagonists or partial antagonists of RIPK2 having a structure of Formula (VIII) or a pharmaceutically acceptable salt or isotopic variant thereof:

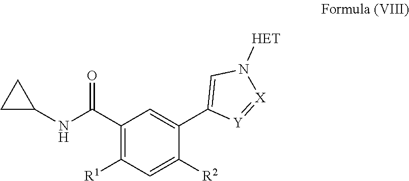

wherein:
HET is

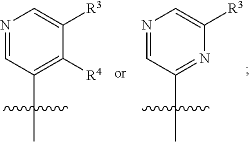

X is N and Y is CH; or
   X is CH and Y is N;
R¹ is —H, or —F;
R² is C₁₋₃alkyl, —Cl, or —F;
R³ and R⁴ are each independently —H; —OR⁵; —O—C₁₋₆alkyl-O—C₁₋₃alkyl; —O—C₃₋₆cycloalkyl; —C(O)R⁵, C₁₋₆alkyl optionally substituted with one to three —OH, —F, C₃₋₈heterocycloalkyl optionally substituted with oxo, C₃₋₆cycloalkyl, —C(O)OR⁵, —O—C₁₋₆alkyl, aryl, —N(R⁵)(R⁶), —CN, or —C(O)N(R⁵)(R⁶); C₃₋₆cycloalkyl optionally substituted with one to three —OH, one to three —F, C₁₋₆alkyl, —O—C₁₋₆alkyl, C₁₋₆alkyl-OC₁₋₆alkyl, C₁₋₆alkyl-OH, —CF₃, —CN, —OC₃₋₆cycloalkyl, —C(O)OH, —C(O)OR⁵, C₃₋₆cycloalkyl, 5-6 membered heteroaryl, C₃₋₆ heterocycloalkyl, N(R⁵)(R⁶), or —C(O)N(R⁵)(R⁶); —C(O)OR⁵; —C(O)N(R⁵)(R⁶); —S(═O)2N(R⁵)(R⁶); —S(O)ₙ—R⁵; a 4-10 membered monocyclic, bicyclic, or spirocyclic heterocyclyl group containing nitrogen, sulfur, or oxygen and optionally substituted with one to three —N(R⁵)(R⁶), halogen, —C₁₋₆alkyl, —O—C₁₋₆alkyl, or —C₁₋₆haloalkyl; aryl; —N(R⁵)(R⁶); or halogen;
R⁵ and R⁶ are each independently —H; -C₁₋₆alkyl-C₃₋₈heterocycloalkyl; a 4-6 membered heterocycloalkyl wherein the heterocycloalkyl ring is optionally substituted with one to three C₁₋₆alkyl, —OC₁₋₆alkyl, —C₁₋₆haloalkyl, C₁₋₆cycloalkyl, halogen, acyl, heterocycloalkyl, heterocycloalkyl-C₁₋₆alkyl, heterocycloalkyl-O—C₁₋₆alkyl, heterocycloalkyl-OH, heterocycloalkyl-C(O)CH₃, heterocycloalkyl-C(O)OC₁₋₃alkyl, —C₁₋₆alkyl-heterocyclyoakyl, —C₁₋₆alkyl-heterocycloalkyl-C₁₋₆alkyl, —C₁₋₆alkyl-OH, —C₁₋₆alkyl-O—C₁₋₆alkyl, C₃₋₆cycloalkyl, —C₁₋₆alkyl-cycloalkyl, C₃₋₆cycloalkyl-C₁₋₆alkyl, C₃₋₆cycloalkyl-O—C₁₋₆alkyl, or C₃₋₆cycloalkyl-O—C₁₋₆alkyl-OH; acyl; C₃₋₆cycloalkyl-C(O)—C₁₋₃alkyl; —C(O)—C₁₋₃alkyl-O—CH₃; —C(O)—C₁₋₃alkyl; —C(O)—C₃₋₆cycloalkyl; —C(O)—NH—C₁₋₃alkyl; —C(O)—NH—C₁₋₃alkyl; —C(O)—NH—C₃₋₆cycloalkyl optionally monosubstituted or disubstituted with —C₁₋₃alkyl-OH, —C(O)—NH—C₃₋₆heterocyclyl, —C(O)-aryl, —C(O)-heteroaryl, or —S(O)ₙ—C₁₋₃alkyl; and C₁₋₆alkyl optionally substituted with —OH, —O—C₁₋₃alkyl, C₃₋₆cycloalkyl, heterocyclyl, aryl, —NH—C₁₋₃alkyl, or —N—(C₁₋₃alkyl)₂; or
   R⁵ and R⁶ together with the nitrogen atom to which they are attached form a 5-6 membered heterocyclic ring optionally substituted with methyl; and
n is 0, 1, or 2.

In some embodiments, for a compound of Formula (VIII), HET is

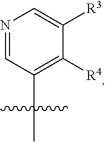

X is N, Y is CH, and n is 1 or 2. In some embodiments, for a compound of Formula (VIII), HET is

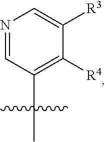

X is N, Y is CH, R² is —CH₃ or —Cl, R⁴ is H, and n is 2. In some embodiments, for a compound of Formula (VIII), HET is

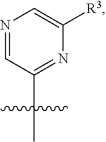

X is N, Y is CH, R² is —CH₃ or —Cl, and n is 2. In some embodiments, for a compound of Formula (VIII), HET is

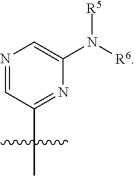

In some embodiments, for a compound of Formula (VIII), HET is

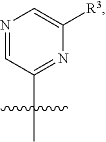

X is CH, Y is N, R² is —CH₃ or —Cl, and n is 2. In some embodiments, for a compound of Formula (VIII), HET is

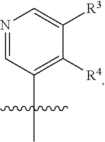

X is CH, Y is N, R² is —CH₃ or —Cl, R⁴ is —H, and n is 2. In some embodiments, for a compound of Formula (VIII), HET is

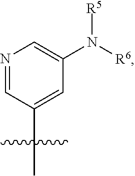

X is CH, Y is N, R² is —CH₃ or —Cl, R⁴ is —H, and n is 2.

In some embodiments, for a compound of Formula (VIII), HET is

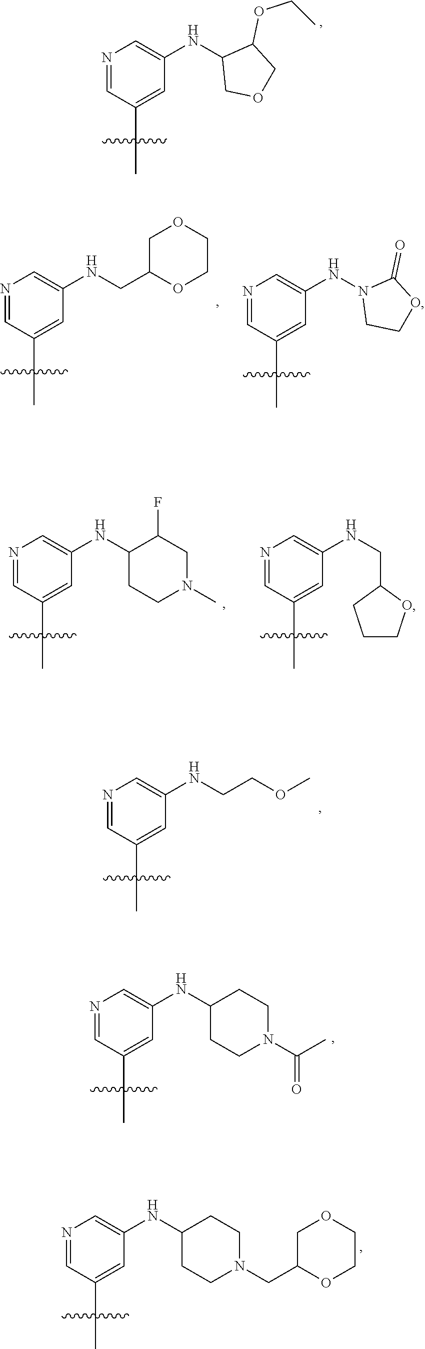

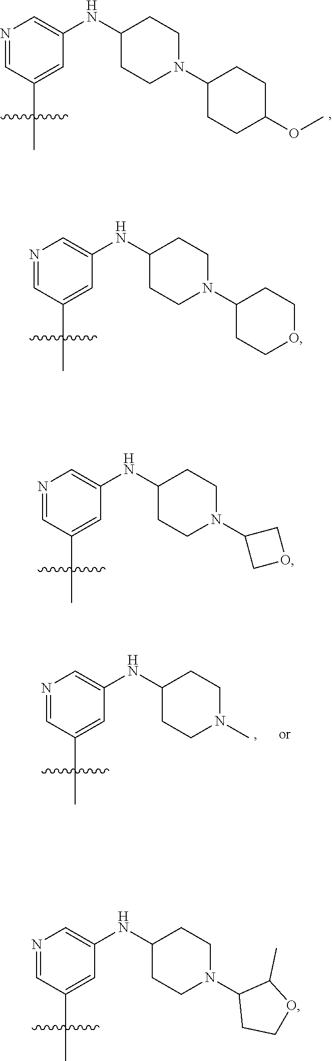 [SMILES: *c1cncc(NC2CCN(C3CCC(OC)CC3)CC2)c1 ; *c1cncc(NC2CCN(C3CCOCC3)CC2)c1 ; *c1cncc(NC2CCN(C3COC3)CC2)c1 ; *c1cncc(NC2CCN(C)CC2)c1 ; *c1cncc(NC2CCN(C3CCOC3C)CC2)c1]

X is N, Y is CH, R¹ is —F, and R² is —CH₃. In some embodiments, for a compound of Formula (VIII), HET is

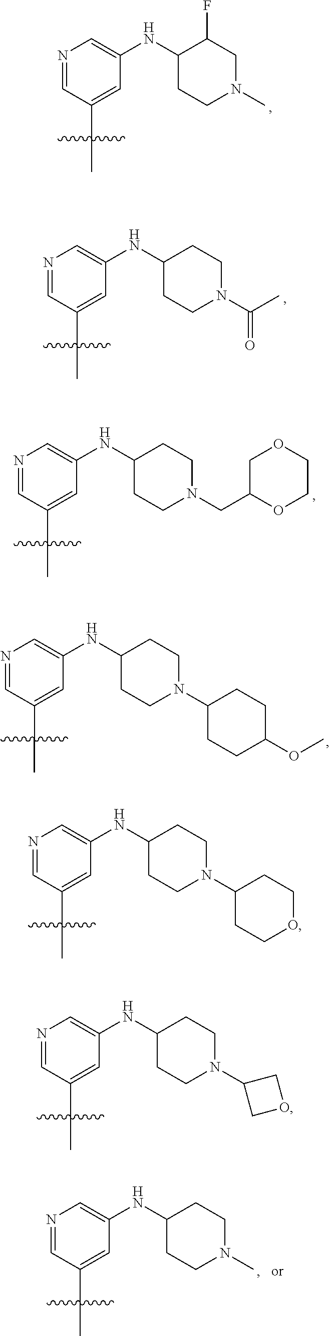

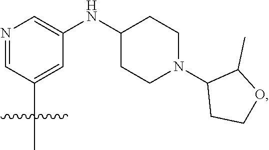 [SMILES: *c1cncc(NC2CCN(C3CCOC3C)CC2)c1]

X is N, Y is CH, R¹ is —F, and R² is —CH₃. In some embodiments, for a compound of Formula (VIII), HET is

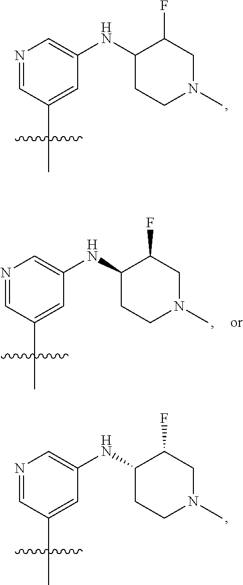 [SMILES: *c1cncc(NC2CCN(C)CC2F)c1 ; *c1cncc(N[C@@H]2CCN(C)C[C@@H]2F)c1 ; *c1cncc(N[C@H]2CCN(C)C[C@H]2F)c1]

X is N, Y is CH, R¹ is —F, and R² is —CH₃.

In some embodiments, for a compound of Formula (VIII), HET is

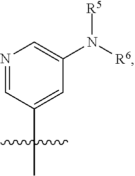

X is N, Y is CH, R² is —CH₃ or —Cl, R⁴ is H, and n is 2. In some embodiments, for a compound of Formula (VIII), HET is

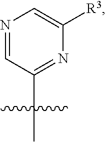

X is N, and Y is CH. In some embodiments, for a compound of Formula (VIII), HET is

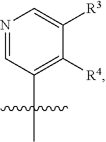

X is CH, and Y is N. In some embodiments, for a compound of Formula (VIII), HET is

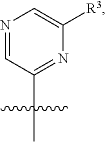

### X is CH, and Y is N.

In some embodiments, for a compound of Formula (VIII), HET is

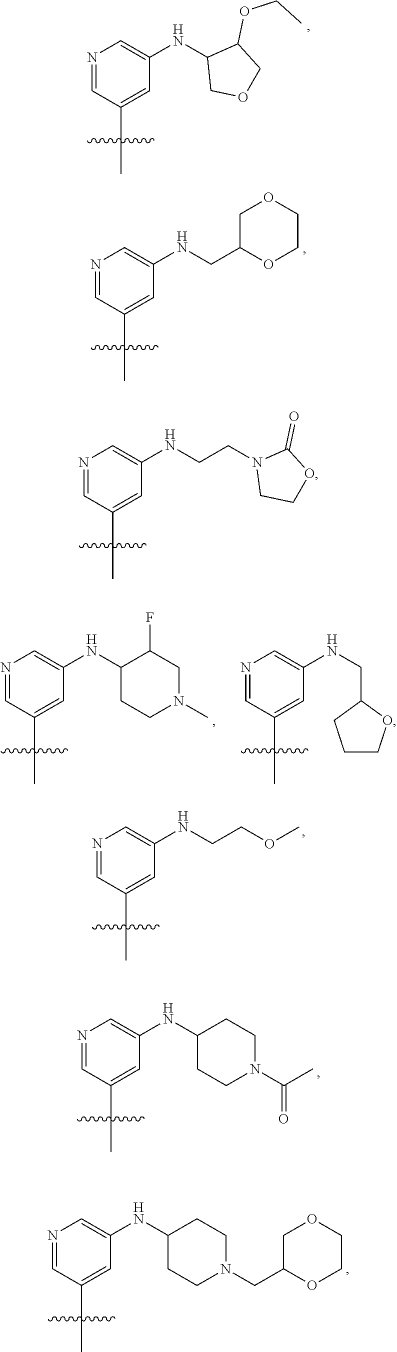

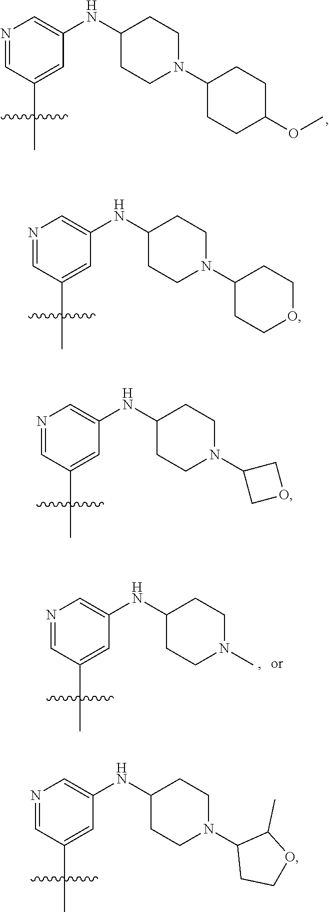 [SMILES: *c1cncc(NC2CCN(C3CCC(OC)CC3)CC2)c1 ; *c1cncc(NC2CCN(C3CCOCC3)CC2)c1 ; *c1cncc(NC2CCN(C3COC3)CC2)c1 ; *c1cncc(NC2CCN(C)CC2)c1 ; *c1cncc(NC2CCN(C3CCOC3C)CC2)c1]

X is N, Y is CH, R² is —CH₃ or —Cl, R⁴ is H, and n is 2.

In some embodiments, a compound of Formula (VIII), or a pharmaceutically acceptable salt or isotopic variant thereof, has the structure of:

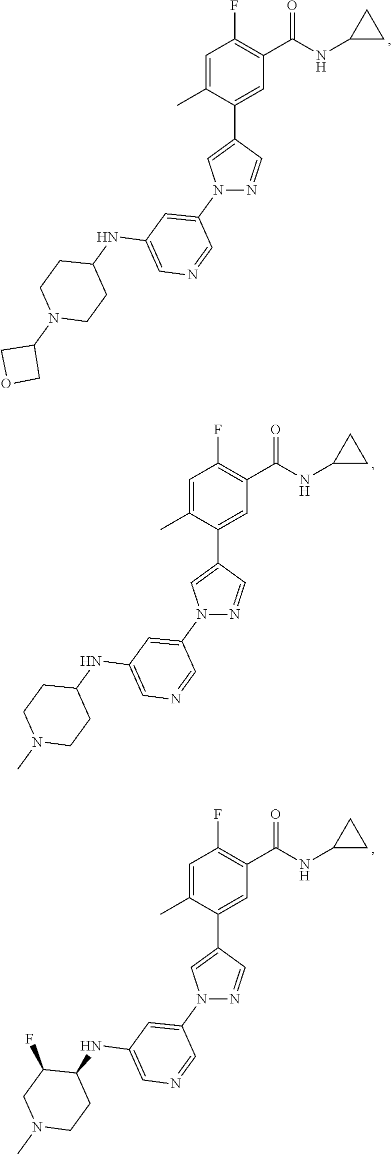 [SMILES: Cc1cc(F)c(C(=O)NC2CC2)cc1-c1cnn(-c2cncc(NC3CCN(C4COC4)CC3)c2)c1 ; Cc1cc(F)c(C(=O)NC2CC2)cc1-c1cnn(-c2cncc(NC3CCN(C)CC3)c2)c1 ; Cc1cc(F)c(C(=O)NC2CC2)cc1-c1cnn(-c2cncc(N[C@H]3CCN(C)C[C@H]3F)c2)c1]

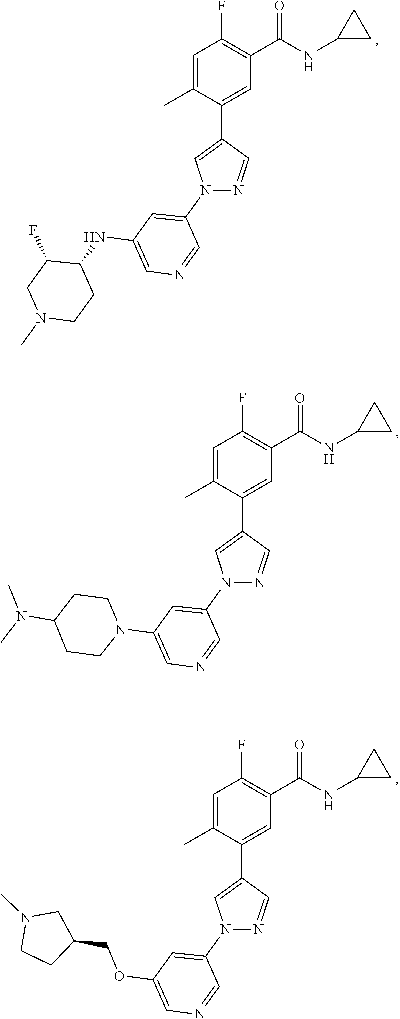 [SMILES: Cc1cc(F)c(C(=O)NC2CC2)cc1-c1cnn(-c2cncc(N[C@@H]3CCN(C)C[C@@H]3F)c2)c1 ; Cc1cc(F)c(C(=O)NC2CC2)cc1-c1cnn(-c2cncc(N3CCC(N(C)C)CC3)c2)c1 ; Cc1cc(F)c(C(=O)NC2CC2)cc1-c1cnn(-c2cncc(OC[C@H]3CCN(C)C3)c2)c1]

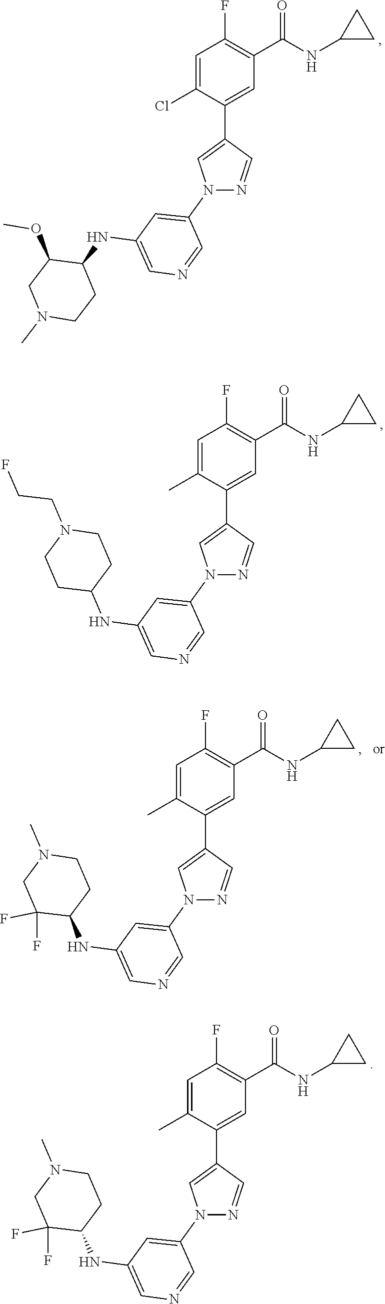 [SMILES: CO[C@@H]1CN(C)CC[C@@H]1Nc1cncc(-n2cc(-c3cc(C(=O)NC4CC4)c(F)cc3Cl)cn2)c1 ; Cc1cc(F)c(C(=O)NC2CC2)cc1-c1cnn(-c2cncc(NC3CCN(CCF)CC3)c2)c1 ; Cc1cc(F)c(C(=O)NC2CC2)cc1-c1cnn(-c2cncc(N[C@@H]3CCN(C)CC3(F)F)c2)c1 ; Cc1cc(F)c(C(=O)NC2CC2)cc1-c1cnn(-c2cncc(N[C@H]3CCN(C)CC3(F)F)c2)c1]

Disclosed herein, in some embodiments, are antagonists or partial antagonists of RIPK2 having a structure of Formula (IX), or a pharmaceutically acceptable salt or isotopic variant thereof:

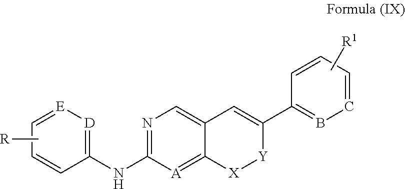

wherein
R is —H; or
R is

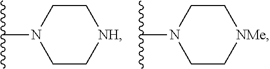

S(═O)₂CH₃,

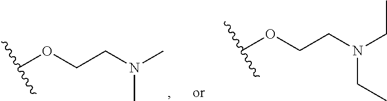

at one available ring position;
A and D are independently N or CH;
E is N, CH, or CR;
B and C are independently N, CH, or C—Cl;
R¹ is H; or
R¹ is C—Cl, C—F, C—OCH₃, C—C(CH₃)₃, or C—OH at one available ring position; and
X—Y are C═C or

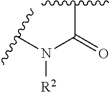

wherein R² is —H, C₁₋₆alkyl, C₁₋₆alkyl-OH, C₁₋₆alkyl-OC₁₋₆alkyl, or C₁₋₆alkyl-aryl.

In some embodiments, for a compound of Formula (IX), R² is methyl, ethyl, isobutyl, 2-hydroxyethyl, 2-methoxyethyl, benzyl, or phenethyl. In some embodiments, for a compound of Formula (IX), R² is methyl. In some embodiments, for a compound of Formula (IX), R² is ethyl. In some embodiments, for a compound of Formula (IX), R² is isobutyl. In some embodiments, for a compound of Formula (IX), R² is 2-hydroxyethyl. In some embodiments, for a compound of Formula (IX), R² is 2-methoxyethyl. In some embodiments, for a compound of Formula (IX), R² is benzyl. In some embodiments, for a compound of Formula (IX), R² is phenethyl.

In some embodiments, a compound of Formula (IX), or a pharmaceutically acceptable salt and isotopic variant thereof, has the structure of:

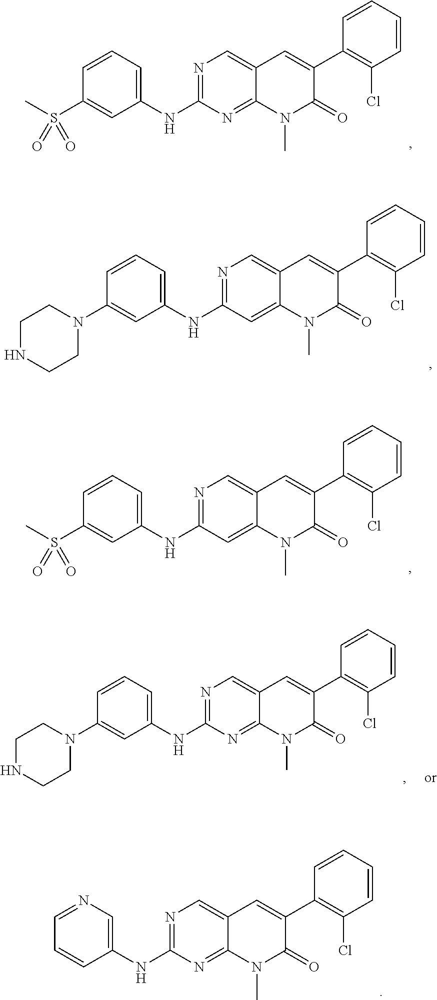 [SMILES: Cn1c(=O)c(-c2ccccc2Cl)cc2cnc(Nc3cccc(S(C)(=O)=O)c3)nc21 ; Cn1c(=O)c(-c2ccccc2Cl)cc2cnc(Nc3cccc(N4CCNCC4)c3)cc21 ; Cn1c(=O)c(-c2ccccc2Cl)cc2cnc(Nc3cccc(S(C)(=O)=O)c3)cc21 ; Cn1c(=O)c(-c2ccccc2Cl)cc2cnc(Nc3cccc(N4CCNCC4)c3)nc21 ; Cn1c(=O)c(-c2ccccc2Cl)cc2cnc(Nc3cccnc3)nc21]

Disclosed herein, in some embodiments, are antagonists or partial antagonists of RIPK2 having a structure of Formula (II(a) or a pharmaceutically acceptable salt and isotopic variant thereof:

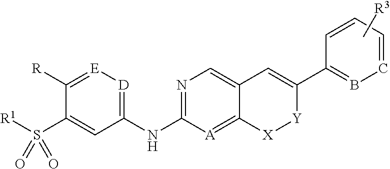

wherein
R is —H,

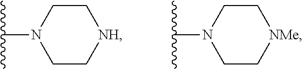 [SMILES: *N1CCNCC1 ; *N1CCN(C)CC1]

S(═O)₂CH₃,

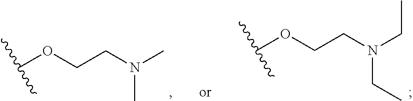

R¹ is C₁₋₆alkyl or 6- to 10-membered aryl;
A and D are independently N or CH;
E is N, CH, or CR;
B and C are independently N, CH, or C—Cl;
R³ is H; or
R³ is C—Cl, C—F, C—OCH₃, C—C(CH₃)₃, or C—OH at one available ring position; and X—Y are C═C or

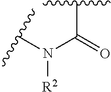

wherein R² is —H, C₁₋₆alkyl, C₁₋₆alkyl-OH, C₁₋₆alkyl-OC₁₋₆alkyl, or C₁₋₆alkyl-aryl.

In some embodiments, for a compound of Formula (IXa), R¹ is methyl, ethyl, or propyl. In some embodiments, for a compound of Formula (IXa), R¹ is methyl. In some embodiments, for a compound of Formula (IXa), R¹ is ethyl. In some embodiments, for a compound of Formula (IXa), R¹ is propyl.

In some embodiments, for a compound of Formula (IXa), R² is methyl, ethyl, isobutyl, 2-hydroxyethyl, 2-methoxyethyl, benzyl, or phenethyl. In some embodiments, for a compound of Formula (IXa), R² is methyl. In some embodiments, for a compound of Formula (IXa), R² is ethyl. In some embodiments, for a compound of Formula (IXa), R² is isobutyl. In some embodiments, for a compound of Formula (IXa), R² is 2-hydroxyethyl. In some embodiments, for a compound of Formula (IXa), R² is 2-methoxyethyl. In some embodiments, for a compound of Formula (IXa), R² is benzyl. In some embodiments, for a compound of Formula (IXa), R² is phenethyl.

Disclosed herein, in some embodiments, are antagonists or partial antagonists of RIPK2 having a structure of Formula (X), or a pharmaceutically acceptable salt or isotopic variant thereof:

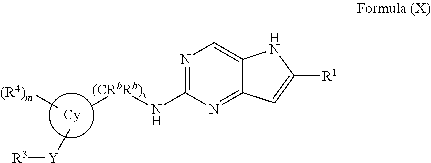

wherein
Cy is C₃₋₈cycloalkyl, C₂₋₉heterocycloalkyl, 6- to 10-membered aryl, or C₂₋₉heteroaryl;
Y is absent, —CR^{b}R^{b}—, —O—, or —S(O)ₙ—;
R¹ is C₃₋₈cycloalkyl, C₂₋₉heterocycloalkyl, 6- to 10-membered aryl, or C₂₋₉heteroaryl, each of which is optionally substituted with one to three R^{a};
R³ is —H, C₂₋₉heterocycloalkyl, or C₂₋₉heteroaryl, wherein the heterocycloalkyl and heteroaryl are optionally substituted with one to three —F, —Cl, —Br, I, —CN, —NO₂, —OR^{b}, C₁₋₄alkyl, —₁₋₃alkyl-OR^{b}, —C₁₋₃alkyl-NR^{b}R^{b}, C₁₋₄haloalkyl, C₁₋₄haloalkoxy, C₃₋₈cycloalkyl, —NR^{b}R^{b}, —C(O)NR^{b}R^{b}, —NR^{b}C(O)NR^{b}R^{b}, —S(O)ₙNR^{b}R^{b}, C(O)OR^{b}, —OC(O)OR^{b}, —S(O)ₙR^{b}, —NR^{b}S(O)ₙR^{b}, —C(S)OR^{b}, —OC(S)R^{b}, —NR^{b}C(O)R^{b}, —C(S)NR^{b}R^{b}, —NR^{b}C(S)R^{b}, —NR^{b}C(O)OR^{b}, —OC(O)NR^{b}R^{b}, —NR^{b}C(S)OR^{b}, —OC(S)NR^{b}R^{b}, —NRC(S)NR^{b}R^{b}, —C(S)R^{b}, or —C(O)R^{b};
each R⁴ is independently halogen, —CN, —NR^{b}R^{b}, —OR^{b}, C₁₋₄alkyl, —C₁₋₃alkyl—NR^{b}R^{b}, C₁₋₄haloalkyl, or C₁₋₄haloalkoxy;
   each R^{a} is independently —F, —Cl, —Br, I, —CN, OR^{b}, C₂₋₆alkenyl, C₂₋₆alkynyl, C₁₋₄haloalkyl, C₁₋₄haloalkoxy, —C₁₋₃alkyl-OR^{b}, or —C₁₋₃alkyl—NR^{b}R^{b};
   each R^{b} is independently —H or C₁₋₄alkyl;
x is 0, 1, 2, 3, or 4;
each m is independently 0, 1, 2, or 3; and
each n is independently 0, 1, or 2.

Disclosed herein, in some embodiments, are antagonists or partial antagonists of RIPK2 having a structure of Formula (Xa) or a pharmaceutically acceptable salt or isotopic variant thereof:

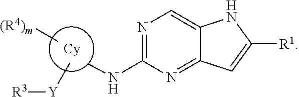

Disclosed herein, in some embodiments, are antagonists or partial antagonists of RIPK2 having a structure of Formula (Xb) or a pharmaceutically acceptable salt or isotopic variant thereof:

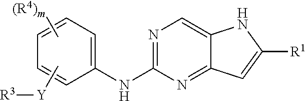

In some embodiments, for a compound of Formula (X), R¹ is optionally substituted phenyl, optionally substituted cyclopentyl, optionally substituted cyclohexyl, optionally substituted thienyl, optionally substituted pyridinyl, optionally substituted thiazolyl, optionally substituted pyrrolyl, optionally substituted imidazolyl, optionally substituted furanyl, optionally substituted oxazolyl, optionally substituted isoxazolyl, optionally substituted pyrazolyl, optionally substituted isothiazolyl, optionally substituted pyrmidinyl, optionally substituted pyrazinyl, optionally substituted pyridazinyl, optionally substituted oxadiazolyl, optionally substituted tetrahydropyranyl, optionally substituted triazolyl, or optionally substituted thiadiazolyl. In some embodiments, for a compound of Formula (X), R¹ is optionally substituted phenyl, optionally substituted cyclopentyl, optionally substiuted thienyl, or optionally substituted tetrahydropyranyl. In some embodiments, for a compound of Formula (X), R¹ is optionally substituted phenyl. In some embodiments, for a compound of Formula (X), R¹ is optionally substituted cyclopentyl. In some embodiments, for a compound of Formula (X), R¹ is optionally substituted thienyl. In some embodiments, for a compound of Formula (X), R¹ is optionally substituted tetrahydropyranyl.

In some embodiments, for a compound of Formula (X), R³ is optionally substituted monocyclic heterocycloalkyl or optionally substituted monocyclic heteroaryl. In some embodiments, for a compound of Formula (X), R³ is optionally substituted monocyclic heterocycloalkyl. In some embodiments, for a compound of Formula (X), R³ is optionally substituted monocyclic heterocycloaryl.

In some embodiments, for a compound of Formula (X), m is 0 to 3. In some embodiments, for a compound of Formula (X), m is 0. In some embodiments, for a compound of Formula (X), m is 1. In some embodiments, for a compound of Formula (X), m is 2. In some embodiments, for a compound of Formula (X), m is 3.

In some embodiments, for a compound of Formula (X), R³ is optionally substituted azetidinyl, optionally substituted morpholinyl, optionally substituted piperazinyl, optionally substituted piperidinyl, optionally substituted tetrahydropyranyl, optionally substituted pyrrolidinyl, optionally substituted thiomorpholinyl, optionally substituted tetrahydrofuryanyl, optionally substituted homomorpholinyl, optionally substituted homopiperazinyl, optionally substituted thiomorpholine dioxide, or optionally substituted thienomorpholine oxide. In some embodiments, for a compound of Formula (X), R³ is optionally substituted morpholinyl, optionally substituted piperazinyl, optionally substituted piperidinyl, or optionally substituted thiomorpholinyl. In some embodiments, for a compound of Formula (X), R³ is optionally substituted morpholinyl. In some embodiments, for a compound of Formula (X), R³ is optionally substituted piperazinyl. In some embodiments, for a compound of Formula (X), R³ is optionally substituted piperidinyl. In some embodiments, for a compound of Formula (X), R³ is optionally substituted thiomorpholinyl.

Disclosed herein, in some embodiments, are antagonists or partial antagonists of RIPK2 having a structure of Formula (Xc) or a pharmaceutically acceptable salt or isotopic variant thereof:

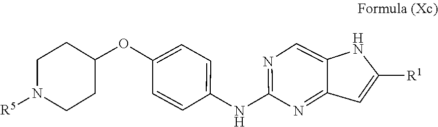

wherein

R⁵ is C₁₋₄alkyl or —C₁₋₃alkyl-OR^{b}.

Disclosed herein, in some embodiments, are antagonists or partial antagonists of RIPK2 having a structure of Formula (Xd) or a pharmaceutically acceptable salt or isotopic variant thereof:

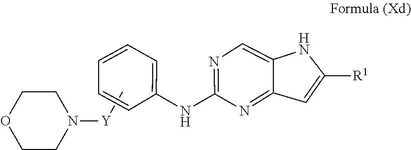

wherein:

Y is absent or —CH₂—; and

Y is attached to the meta or para position of the phenyl ring.

Disclosed herein, in some embodiments, are antagonists or partial antagonists of RIPK2 having a structure of Formula (Xe) or a pharmaceutically acceptable salt or isotopic variant thereof:

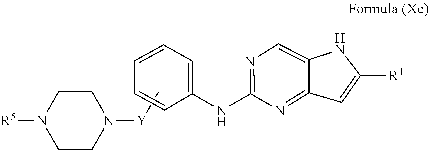

wherein

R⁵ is —H, C₁₋₄alkyl, or —C₁₋₃alkyl-OR^{b};

Y is absent or —CH₂—; and

Y is attached to the meta or para position of the phenyl ring.

In some embodiments, for a compound of Formula (X), R¹ is

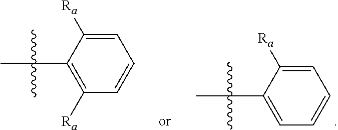

In some embodiments, for a compound of Formula (X), R¹ is

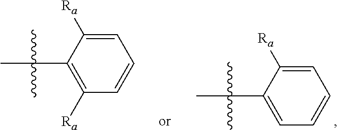

wherein each R^{a} is independently —F, —Cl, or —CH₃.

Dosages and Routes of Administration

In general, methods disclosed herein comprise administering a therapeutic agent by oral administration. However, in some instances, methods comprise administering a therapeutic agent by intraperitoneal injection. In some instances, methods comprise administering a therapeutic agent in the form of an anal suppository. In some instances, methods comprise administering a therapeutic agent by intravenous (“i.v.”) administration. It is conceivable that one may also administer therapeutic agents disclosed herein by other routes, such as subcutaneous injection, intramuscular injection, intradermal injection, trasdsermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., step-wise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 pmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

Additional Therapeutic

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. In some embodiments, the NK cells and CAR-NK cells comprise a cytokine receptor and a suicide gene. In some embodiments, the cell-based therapy comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the additional therapeutic agent comprises an agonist of TL1A, JAK1, GPR35, ADCY7, IFNG, TNFSF8, PFKFB3, SKAP2 GPR65, SPRED2, IL18R1, GSDMB, and gene expression products from genes implicated in the pathogenesis of inflammatory, fibrotic, or fibrostenotic disease. The therapeutic agent may be an allosteric modulator of TL1A, JAK1, GPR35, ADCY7, IFNG, TNFSF8, PFKFB3, SKAP2 GPR65, SPRED2, IL18R1, GSDMB, and gene expression products from genes implicated in the pathogenesis of inflammatory, fibrotic, or fibrostenotic disease.

In some embodiments, the additional therapeutic agent comprises an antagonist. The antagonist may comprise an inhibitor of the activity or expression of TL1A, JAK1, GPR35, ADCY7, IFNG, TNFSF8, PFKFB3, SKAP2 GPR65, SPRED2, IL18R1, GSDMB, and gene expression products from genes implicated in the pathogenesis of inflammatory, fibrotic, or fibrostenotic disease. Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HC1, Tofacitinib (CP-690550,Tasocitinib), XL019, Pacritinib (SB1518), or ZM 39923 HCl.

In some embodiments the additional therapeutic agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti- TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the TL1A antibody is an anti-TL1A antibody that specifically binds to TL1A.

In some embodiments the additional therapeutic agent comprises an inhibitor of CD30L expression or activity. The inhibitor of CD30L expression or activity may be a direct inhibitor or indirect inhibitor. Non-limiting examples of an inhibitor of CD30L expression include RNA to protein TL1A translation inhibitors, antisense oligonucleotides targeting the mRNA (such as miRNAs, or siRNA), epigenetic editing (such as targeting the DNA-binding domain of CD30L, or post-translational modifications of histone tails and/or DNA molecules). In some embodiments, the CD30L inhibitor is an anti-CD30L antibody. The anti-CD30L antibody may be monoclonal or polyclonal. The anti-CD30L antibody may be humanized or chimeric.

In some instances, the additional therapeutic agent comprises administering to the subject a active agent that modulates CARD9 activity or expression. In various embodiments, the inhibitor of CARD9 activity or expression comprises a CARD9 antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-ripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9 a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction, or a combination thereof. In some other embodiments, the inhibitor of CARD9 activity or expression comprises the small molecule inhibitor BRD5529, BRD4203, BRD8991, BRD4098 or a combination thereof In some embodiments, the CARD9 antibody recognizes the total CARD9 protein. In other embodiments, the CARD9 antibody recognizes 90%, 80%, 70%, 60%, 50%, 40%, 30%, 20%, or 10% of the total CARD9 protein. In some embodiments, the modulator of CARD9 comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

In some embodiments, the additional therapeutic agent comprises administering to the subject an antibody or antibody fragment, a small molecule, an allosteric modulator, an agonist, an antagonist, a direct modulator of Dectin-1A, an indirect modulator of Dectin-1A, or a combination thereof. In other embodiments, the treatment is an inhibitor of C-type lectin-like receptors. In various embodiments, the agonist is soluble β-glucan antagonist laminarin. In various other embodiments, the antagonist is soluble β-glucan antagonist laminarin. In some embodiments, the antibody binds to the C-type lectin-like receptors. In some embodiments, the Dectin-1 antibody recognizes the total Dectin-1 protein. In other embodiments, the Dectin-1 antibody recognizes 90%, 80%, 70%, 60%, 50%, 40%, 30%, 20%, or 10% of the total Dectine-1A protein. In some embodiments, the modulator of Dectin-1A comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

In some instances, the additional therapeutic agent comprises administering to the subject an antimycotic agent. In some instances, the antimycotic agent comprises an active agent that inhibits growth of a fungus. In some instances, the antimycotic agent comprises an active agent that kills a fungus. In some embodiments, the antimycotic agent comprises polyene, an azole, an echinocandin, an flucytosine, an allylamine, a tolnaftate, or griseofulvin, or a combination thereof. In other embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In some other embodiments, the polyene comprises amphotericin B, nystatin, or natamycin. In yet other embodiments, the echinocandin comprises caspofungin, anidulafungin, or micafungin. In various other embodiments, the allylamine comprises naftifine or terbinafine.

Pharmaceutical Composition

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, intravenous, intraarterial, oral, parenteral, buccal, topical, transdermal, rectal, intramuscular , subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethyl cellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits and Compositions

Compositions

Disclosed herein, in some embodiments, are compositions useful for the detection of a genotype or biomarker in a sample obtained from a subject according to the methods described herein. Aspects disclosed herein provide compositions comprising a polynucleotide sequence comprising at least 10 but less than 50 contiguous nucleotides of any one of SEQ ID NOS: 12-22, or reverse complements thereof, wherein the contiguous polynucleotide sequence comprises a detectable molecule. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 12. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 13. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 3. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 14. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 15. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 16. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 17. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 18. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 19. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 20. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 21. In some embodiments, the polynucleotide sequence comprises at least about 70%, about 75%, about 80%, about 85%, about 90%, about 91%, about 92%, about 93%, about 94%, about 95%, about 96%, about 97%, about 98%, about 99%, or about 100% sequence identity to the polynucleotide sequence provided in SEQ ID NO: 22.

Aspects disclosed herein also provide compositions comprising a contiguous polynucleotide sequence specific to the risk allele in a single nucleotide polymorphism (SNP), wherein the contiguous polynucleotide sequence comprises a detectable molecule. In some embodiments, the SNP comprises rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination therewith. In some embodiments, the SNP at rs11564258 comprises an “A” or a “G” risk allele. In some embodiments, the SNP at rs2357623 comprises an “A” or a “G” risk allele. In some embodiments, the SNP at rs2066845 comprises a “G” or a “C” risk allele. In some embodiments, the SNP at rs5743289 comprises an “A” or a “G” risk allele. In some embodiments, the SNP at rs72796367 comprises a “G” or an “A” risk allele. In some embodiments, the SNP at rs6752107 comprises a “G” or an “A” risk allele. In some embodiments, the SNP at rs12994997 comprises a “G” or an “A” risk allele. In some embodiments, the SNP at rs11741861 comprises a “G” or an “A” In some embodiments, the SNP at rs9494844 comprises an “A” or a “C” risk allele. In some embodiments, the SNP at rs6918329 comprises a “G” or an “A” risk allele. In some embodiments, the SNP at rs7404095 comprises an “A” or a “G” risk allele. In various embodiments, the detectable molecule comprises a fluorophore. In other embodiments, the polynucleotide sequences further comprise a quencher.

Also disclosed herein, in some embodiments, are compositions comprising an antibody or antigen-binding fragment that specifically binds to RIPK2, wherein the antibody or antigen-binding fragment comprises a detectable molecule. In various embodiments, the antibody comprises a monoclonal antibody, a chimeric antibody, a CDR-grafted antibody, a a Fab, a Fab′, a F(ab′)₂, a Fv, a disulfide linked Fv, a scFv, a single domain antibody, a diabody, a multispecific antibody, a dual specific antibody, an anti-idiotypic antibody, or a bispecific antibody. In some embodiments, the antibody or antigen-binding fragment comprises an IgG antibody, an IgM antibody, and/or an IgE antibody. In some embodiments, the detectable molecule comprises a fluorophore. In some embodiments, the antibody or antigen-binding fragment is conjugated to a paramagnetic particle (e.g., bead).

Kits

Disclosed herein, in some embodiments, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Aspects disclosed herein provide kits comprising (i) a composition comprising a contiguous polynucleotide sequence specific to the risk allele at rs11564258, rs2357623, rs2066845, rs5743289, rs72796367, rs6752107, rs12994997, rs11741861, rs9494844, rs6918329, rs7404095, or a SNP in linkage disequilibrium (LD) therewith, or any combination thereof, wherein the contiguous polynucleotide sequence comprises a detectable molecule; and (ii) a primer pair capable of amplifying at least about 10 contiguous nucleobases comprising the risk allele within SEQ ID NOS 1-11. In some embodiments, the SNP at rs11564258 comprises an “A” or a “G” risk allele. In some embodiments, the SNP at rs2357623 comprises an “A” or a “G” risk allele. In some embodiments, the SNP at rs2066845 comprises a “G” or a “C” risk allele. In some embodiments, the SNP at rs5743289 comprises an “A” or a “G” risk allele. In some embodiments, the SNP at rs72796367 comprises a “G” or an “A” risk allele. In some embodiments, the SNP at rs6752107 comprises a “G” or an “A” risk allele. In some embodiments, the SNP at rs12994997 comprises a “G” or an “A” risk allele. In some embodiments, the SNP at rs11741861 comprises a “G” or an “A” In some embodiments, the SNP at rs9494844 comprises an “A” or a “C” risk allele. In some embodiments, the SNP at rs6918329 comprises a “G” or an “A” risk allele. In some embodiments, the SNP at rs7404095 comprises an “A” or a “G” risk allele. In various embodiments, the detectable molecule comprises a fluorophore. In other embodiments, the polynucleotide sequences further comprise a quencher. In some embodiments, methods are provided for contacting DNA from a subject with the composition described herein, or using the kit described herein under conditions configured to hybridize the composition to the DNA if the DNA comprises a sequence complementary to the composition. In further embodiments, provided herein are methods of treating the subject with antagonist of RIPK2 activity or expression, provided that the DNA from the subject comprises the sequence complementary to the composition. The therapy may include an inhibitor of RIPK2 activity or expression. The inhibitor of RIPK2 activity or expression may comprise one or more molecules from any one of Formulas I-X.

Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., IBD, CD, UC) in a subject. In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with an antagonist of RIPK2, such as those disclosed herein.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

Disclosed herein, in some embodiments, is a system for detecting a particular genotype in a subject. The system is configured to implement the methods described in this disclosure, including, but not limited to, treating a disease or condition in a subject with an antagonist o RIPK2 activity or expression, determine whether the subject is suitable for treatment with an antagonist of RIPK2 activity or expression, and/or diagnosing or prognosing a subject with the disease or condition, or a subtype of the disease or condition. In some instances, the disease or condition comprises inflammatory bowel disease (IBD), Crohn's disease (CD), and/or ulcerative colitis (UC).

In some embodiments, disclosed herein is a system for detecting a genotype in a subject, comprising: (a) a computer processing device, optionally connected to a computer network; and (b) a software module executed by the computer processing device to analyze a target nucleic acid sequence of the genotype in a sample from a subject. In some instances, the genotype comprises one or more single nucleotide polymorphisms (SNPs). In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

In some embodiments, the genotype of the subject comprises one or more single nucleotide polymorphisms (SNPs). In some embodiments, systems described herein utilize at least 1 SNP, about 2 SNPs, at least about 3 SNPs, at least about 4 SNPs, at least about 5 SNPs, at least about 6 SNPs, at least about 7 SNPs, at least about 8 SNPs, at least about 9 SNPs, at least about 10 SNPs, at least about 11 SNPs, at least about 12 SNPs, at least about 13 SNPs, at least about 14 SNPs, at least about 15 SNPs, at least about 20 SNPs, at least about 25 SNPs, at least about 30 SNPs, at least about 40 SNPs, or at least about 50 SNPs. In some embodiments, the one or more SNPs are located at a gene comprising LRRK, NOD2, ATG16L1, SNF300, OLIG3, and/or PRKCB1. Non-limiting examples of SNPs located at the gene comprising LRRK, NOD2, ATG16L1, SNF300, OLIG3, and/or PRKCB1 LRRK, NOD2, ATG16L1, SNF300, OLIG3, and/or PRKCB include SNPs listed in Tables 2-4. In some embodiments, the genotype comprises one or more SNPs selected from Table 1. In some embodiments, the SNPs located at the genes described herein are associated with a likelihood that a subject is suitable for treatment of a disease or disorder with an antagonist of RIPK2 activity of expression, such as those described herein.

In some embodiments, the one or more SNPs is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰. In some embodiments, the one or more SNPs is associated with a risk that the subject has, or will develop, a subclinical phenotype (e.g., stricturing and/or penetrating disease) of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the P value is between about 1.0×10⁻⁶ and about 1.0×10⁻¹⁰⁰. In some embodiments, the P value is above about 1.0×10⁻¹⁰⁰.

In some embodiments, the genotype comprises one or more SNPs in linkage disequilibrium with rs5743289 as determined by an r² value of at least about 0.80, about 0.85, about 0.90, about 0.95, or about 1.0. In some instances, the r² value is 0.80. In some instances, the r² value is 0.85. In some instances, the r² value is 0.90. In some instances, the r² value is 0.95.

In some instances, the r² value is 1.0. In some embodiments, the one or more SNPs comprises rs11564258, rs2357623, rs2066845, rs5743289, rs72796367, rs6752107, rs12994997, rs11741861, rs9494844, rs6918329, rs7404095, a SNP in linkage disequilibrium (LD) therewith, or any combination thereof. In some embodiments, the SNP at rs11564258 comprises an “A” or a “G” allele. In some embodiments, the SNP at rs2357623 comprises an “A” or a “G” allele. In some embodiments, the SNP at rs2066845 comprises a “G” or a “C” allele. In some embodiments, the SNP at rs5743289 comprises an “A” or a “G” allele. In some embodiments, the SNP at rs72796367 comprises a “G” or an “A” allele. In some embodiments, the SNP at rs6752107 comprises a “G” or an “A” allele. In some embodiments, the SNP at rs12994997 comprises a “G” or an “A” allele. In some embodiments, the SNP at rs11741861 comprises a “G” or an “A” In some embodiments, the SNP at rs9494844 comprises an “A” or a “C” allele. In some embodiments, the SNP at rs6918329 comprises a “G” or an “A” allele. In some embodiments, the SNP at rs7404095 comprises an “A” or a “G” allele. In some instances, the SNP is within a polynucleotide sequence provided in SEQ ID NOS: 1-11.

One feature of a computer program includes a sequence of instructions, executable in the digital processing device's CPU, written to perform a specified task. In some embodiments, ccomputer readable instructions are implemented as program modules, such as functions, features, Application Programming Interfaces (APIs), data structures, and the like, that perform particular tasks or implement particular abstract data types. In some embodiments, the computer program is configured to (a) receive data corresponding to a presence or an absence of a genotype of a subject; (b) determine whether the subject is at risk of developing a disease or disorder and/or is suitable for treatment with a modulator of RIPK2 activity or expression. In some embodiments, the computer program is trained with plurality of training samples, and wherein the sample from the subject is independent from the plurality of training samples. In some embodiments, the training samples are derived from a reference population of individuals diagnosed with the disease or disorder, and a reference population of individual who are normal (e.g., not diagnosed with, and do not have, the disease or disorder). In some embodiments, (b) comprises calculating a polygenic risk score (PRS). In some embodiments, the PRS comprises a normalized weighted sum of a number of risk alleles within the genotype present in the subject with weights proportional to a beta value of association between the genotype with the disease or condition. To the extent an absence of a genotype is detected, the systems disclosed herein further comprises utilize data corresponding to a presence or an absence of a surrogate genotype to calculate the PRS. In some embodiments, a surrogate genotype is selected if it is linkage disequilibrium (LD) with the absence genotype, as determined by an r² value of at least about, 0.8, about 0.85, about 0.90, about 0.95, or about 1.0.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™ Lisp, Python™, Visual Basic, and VB .NET, or combinations thereof. Compilation may be often performed, at least in part, to create an executable program. In some instances, a computer program includes one or more executable complied applications.

Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML, databases. In some embodiments, a database is internet-based. In some embodiments, a database is web-based. In some embodiments, a database is cloud computing-based. A database may be based on one or more local computer storage devices.

Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

Business Methods Utilizing a Computer

The methods described herein may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a genotype may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### EXAMPLES

### Example 1. SNPs Associated with Inflammatory Bowel Disease, Crohn's Disease, or Ulcerative Colitis Located at IBD Risk Gene Loci

Patients in MIRMAID cohort with IBD were recruited at the Cedars-Sinai Inflammatory Bowel Disease Centers. The diagnosis of each patient was based on standard endoscopic, histologic, and radiographic features. Blood samples were collected from patients at the time of enrollment. Blood samples were also collected from individuals without IBD. Genotyping was performed at Cedars-Sinai Medical Center using Illumina ImmunoChip on all samples collected. Markers were excluded from analysis based on: Hardy-Weinberg Equilibrium p≤1.0E-5; genotyping rate <95%; minor allele frequency <1%. Related individuals (Pi-hat scores >0.25) were identified using identity-by-descent and excluded from analysis (PLINK). Admixture was used to generate ethnicity proportion estimations for all individuals. Only subjects identified by admixture as Caucasian (proportion <0.75) were included in the analysis.

Multiple large-scale case-control association studies involving Inflammatory Bowel Disease (IBD) Crohn's disease (CD), and ulcerative colitis (UC) in Caucasian populations using gene-based single nucleotide polymorphism (SNP) markers were performed. The studies included patients recruited at the Cedars-Sinai Inflammatory Bowel Disease Centers with Inflammatory Bowel Disease (IBD), Crohn's disease (CD)(n=2291), and ulcerative colitis (UC). The studies also included GWAS data derived from the IIBDGC, and Wellcome Trust databases. Table 1 provides meta-analysis of SNPs associated with CD (P value cutoff is 1.0×10⁻⁶). SNPs considered predictive of disease, and/or suitability to treatment with an antagonist of RIPK2 activity or expression were selected based on being located at a gene or gene loci involved in the NOD2/CARD15 or autophagy pathways, including the RIPK2 pathway with a demonstrable strong association with the disease (see Tables 2-4). Table 2 provides SNPs associated with IBD. Table 3 provides SNPs associated with CD. Table 4 provides SNPs associated with UC.

TABLE 1
SNPs Associated with Crohn's Disease (Metadata Analysis)

| | | | Minor | Major | | | P |
|---|---|---|---|---|---|---|---|
| SNP | CHR | Basepair | Allele | Allele | Beta | SE | value |
| imm_16_49302700 | 16 | 50745199.00 | A | G | 0.351704953 | 0.010558797 | 2.84E−243 |
| imm_16_49302125 | 16 | 50744624.00 | A | G | 0.363089391 | 0.010928012 | 4.60E−242 |
| imm_16_49305205 | 16 | 50747704.00 | A | G | 0.351321005 | 0.010574751 | 5.07E−242 |
| imm_16_49300439 | 16 | 50742938.00 | A | G | 0.329669011 | 0.01044575 | 1.30E−218 |
| imm_16_49296331 | 16 | 50738830.00 | A | G | 0.330360207 | 0.010468749 | 1.45E−218 |
| imm_16_49296951 | 16 | 50739450.00 | A | G | 0.330015869 | 0.010469358 | 4.33E−218 |
| imm_16_49300182 | 16 | 50742681.00 | A | G | 0.329209766 | 0.01044582 | 5.26E−218 |
| imm_16_49287730 | 16 | 50730229.00 | A | G | 0.336714707 | 0.010908022 | 3.16E−209 |
| imm_16_49288597 | 16 | 50731096.00 | A | G | 0.321389696 | 0.010487419 | 3.06E−206 |
| imm_16_49290875 | 16 | 50733374.00 | A | C | 0.316204557 | 0.010556927 | 4.10E−197 |
| imm_16_49314382 | 16 | 50756881.00 | G | A | 0.362089426 | 0.014841607 | 1.84E−131 |
| imm_16_49309665 | 16 | 50752164.00 | A | G | 0.360512482 | 0.014846469 | 2.99E−130 |
| imm_16_49314275 | 16 | 50756774.00 | A | G | 0.396201009 | 0.016605787 | 8.13E−126 |
| imm_2_233813122 | 2 | 234148383.00 | A | G | −0.241089795 | 0.010256718 | 3.58E−122 |
| rs2066844 | 16 | 50745926.00 | A | G | 0.671650344 | 0.028943454 | 4.00E−119 |
| imm_16_49303427 | 16 | 50745926.00 | A | G | 0.671362923 | 0.028944046 | 5.09E−119 |
| imm_2_233826322 | 2 | 234161583.00 | A | G | −0.230384063 | 0.009950269 | 1.34E−118 |
| imm_2_233832620 | 2 | 234167881.00 | T | A | −0.229984408 | 0.00994232 | 2.21E−118 |
| imm_2_233831973 | 2 | 234167234.00 | A | G | −0.229803784 | 0.009945637 | 4.03E−118 |
| imm_2_233838242 | 2 | 234173503.00 | G | A | −0.229550418 | 0.0099419 | 5.95E−118 |
| imm_2_233826187 | 2 | 234161448.00 | G | A | −0.229578892 | 0.009946004 | 6.94E−118 |
| imm_2_233826508 | 2 | 234161769.00 | G | A | −0.229493717 | 0.009946738 | 8.79E−118 |
| imm_2_233845149 | 2 | 234180410.00 | A | G | −0.229260596 | 0.009941326 | 1.13E−117 |
| imm_2_233848107 | 2 | 234183368.00 | A | G | −0.229302274 | 0.009945931 | 1.31E−117 |
| imm_2_233846587 | 2 | 234181848.00 | G | A | −0.229270471 | 0.009946215 | 1.44E−117 |
| imm_2_233822427 | 2 | 234157688.00 | G | A | −0.229095706 | 0.009945449 | 2.07E−117 |
| imm_2_233846979 | 2 | 234182240.00 | G | A | −0.229024402 | 0.009946215 | 2.54E−117 |
| imm_2_233840445 | 2 | 234175706.00 | C | A | −0.228763371 | 0.00994106 | 3.54E−117 |
| imm_2_233849156 | 2 | 234184417.00 | G | A | −0.228784231 | 0.00994457 | 4.06E−117 |
| imm_2_233823578 | 2 | 234158839.00 | G | A | −0.228810377 | 0.009946046 | 4.14E−117 |
| imm_2_233843192 | 2 | 234178453.00 | G | A | −0.228565137 | 0.009945124 | 6.95E−117 |
| imm_2_233848207 | 2 | 234183468.00 | G | A | −0.228792141 | 0.009956836 | 7.67E−117 |
| imm_2_233850738 | 2 | 234185999.00 | G | A | −0.228423218 | 0.00994595 | 1.01E−116 |
| imm_2_233841348 | 2 | 234176609.00 | G | A | −0.228075285 | 0.009945329 | 2.18E−116 |
| imm_2_233837585 | 2 | 234172846.00 | A | G | −0.235068613 | 0.010266322 | 4.97E−116 |
| imm_2_233837654 | NA | NA | NA | NA | −0.244093096 | 0.010703864 | 4.17E−115 |
| imm_2_233825084 | 2 | 234160345.00 | A | C | −0.233672073 | 0.010258716 | 7.59E−115 |
| imm_16_49267224 | 16 | 50709723.00 | A | G | −0.39216235 | 0.017235827 | 1.35E−114 |
| imm_2_233848457 | 2 | 234183718.00 | A | G | −0.233413517 | 0.010267442 | 2.10E−114 |
| imm_2_233849691 | NA | NA | NA | NA | −0.243017931 | 0.010691159 | 2.23E−114 |
| imm_2_233844429 | 2 | 234179690.00 | G | A | −0.228267236 | 0.010059807 | 5.50E−114 |
| imm_2_233821275 | 2 | 234156536.00 | C | A | −0.225250862 | 0.009975752 | 6.84E−113 |
| imm_2_233811914 | 2 | 234147175.00 | A | G | −0.225121655 | 0.00999609 | 2.59E−112 |
| imm_2_233812669 | 2 | 234147930.00 | G | A | −0.224280132 | 0.009990664 | 1.31E−111 |
| imm_2_233815770 | 2 | 234151031.00 | G | A | −0.223389186 | 0.009970908 | 3.59E−111 |
| imm_2_233814441 | 2 | 234149702.00 | G | A | −0.22291964 | 0.00996887 | 9.32E−111 |
| imm_2_233811289 | 2 | 234146550.00 | G | A | −0.229828383 | 0.010321715 | 7.81E−110 |
| imm_2_233810683 | 2 | 234145944.00 | A | C | −0.220568906 | 0.009946862 | 6.02E−109 |
| imm_16_49385310 | 16 | 50827809.00 | G | A | 0.621665973 | 0.028060772 | 9.49E−109 |
| imm_2_233871489 | 2 | 234206750.00 | A | C | −0.217516146 | 0.009877476 | 1.80E−107 |
| imm_2_233871349 | 2 | 234206610.00 | G | A | −0.216849842 | 0.009880788 | 9.33E−107 |
| imm_2_233871213 | 2 | 234206474.00 | G | A | −0.216607728 | 0.00987392 | 1.14E−106 |
| imm_2_233872997 | 2 | 234208258.00 | G | A | −0.216710492 | 0.009900173 | 3.27E−106 |
| imm_16_49347659 | 16 | 50790158.00 | C | A | 0.628932562 | 0.028861083 | 2.78E−105 |
| imm_2_233810357 | 2 | 234145618.00 | G | A | −0.217764278 | 0.010056432 | 5.55E−104 |
| imm_16_49282694 | 16 | 50725193.00 | A | G | −0.216153165 | 0.010066839 | 2.86E−102 |
| imm_16_49277244 | 16 | 50719743.00 | A | G | 0.607981054 | 0.028826484 | 9.61E−99 |
| imm_16_49272686 | 16 | 50715185.00 | A | G | −0.20713212 | 0.010092816 | 1.35E−93 |
| imm_16_49323628 | 16 | 50766127.00 | C | A | −0.204988362 | 0.010085716 | 7.79E−92 |
| imm_16_49339303 | 16 | 50781802.00 | G | A | −0.204011012 | 0.010051022 | 1.35E−91 |
| imm_16_49327064 | 16 | 50769563.00 | G | A | -0.20347979 | 0.01004165 | 2.70E−91 |
| imm_16_49356430 | 16 | 50798929.00 | G | A | −0.202669632 | 0.010031707 | 9.23E−91 |
| imm_16_49374720 | 16 | 50817219.00 | G | A | −0.203555663 | 0.010086788 | 1.45E−90 |
| imm_16_49262456 | 16 | 50704955.00 | C | A | 0.591607913 | 0.029325586 | 1.66E−90 |
| imm_16_49294157 | 16 | 50736656.00 | G | A | −0.203226123 | 0.01011763 | 9.72E−90 |
| imm_16_49313210 | 16 | 50755709.00 | C | A | −0.202556823 | 0.010088555 | 1.15E−89 |
| imm_16_49298963 | 16 | 50741462.00 | C | A | −0.202799091 | 0.010135679 | 4.65E−89 |
| imm_16_49377764 | 16 | 50820263.00 | G | A | −0.200755997 | 0.010084592 | 3.52E−88 |
| imm_16_49377579 | 16 | 50820078.00 | A | G | −0.200454973 | 0.010088787 | 7.54E−88 |
| imm_16_49365280 | 16 | 50807779.00 | A | G | −0.19967051 | 0.010061797 | 1.23E−87 |
| imm_16_49301420 | 16 | 50743919.00 | A | G | −0.200045254 | 0.010142459 | 1.35E−86 |
| imm_16_49308899 | 16 | 50751398.00 | G | A | −0.199628638 | 0.01015257 | 4.49E−86 |
| imm_2_233855479 | 2 | 234190740.00 | A | G | −0.19958871 | 0.01015618 | 5.56E−86 |
| imm_16_49310925 | 16 | 50753424.00 | A | G | −0.199570853 | 0.010174028 | 1.14E−85 |
| imm_16_49310316 | 16 | 50752815.00 | G | A | −0.199482238 | 0.010179739 | 1.67E−85 |
| imm_16_49309288 | 16 | 50751787.00 | A | G | −0.199312894 | 0.010179421 | 2.29E−85 |
| imm_16_49300832 | 16 | 50743331.00 | A | G | −0.196593773 | 0.010232319 | 2.88E−82 |
| imm_16_49398985 | 16 | 50841484.00 | A | G | −0.193446861 | 0.010069062 | 2.94E−82 |
| imm_16_49400462 | 16 | 50842961.00 | G | A | −0.192318409 | 0.010089094 | 5.22E−81 |
| imm_16_49303084 | 16 | 50745583.00 | C | A | −0.192837568 | 0.010161297 | 2.61E−80 |
| imm_16_49326763 | 16 | 50769262.00 | C | G | 0.187301648 | 0.009947946 | 4.44E−79 |
| imm_16_49322337 | 16 | 50764836.00 | A | G | −0.191080448 | 0.010193419 | 2.11E−78 |
| imm_16_49251512 | 16 | 50694011.00 | A | G | 0.206419294 | 0.01106061 | 9.98E−78 |
| imm_16_49399296 | 16 | 50841795.00 | A | G | −0.19447062 | 0.010445174 | 2.29E−77 |
| imm_16_49349769 | 16 | 50792268.00 | A | G | −0.187834305 | 0.01014299 | 1.46E−76 |
| imm_16_49284323 | 16 | 50726822.00 | A | G | 0.580450247 | 0.031379771 | 2.16E−76 |
| imm_2_233810786 | 2 | 234146047.00 | C | A | −0.188480358 | 0.010199629 | 3.04E−76 |
| imm_2_233810710 | 2 | 234145971.00 | A | G | −0.188195969 | 0.01020064 | 5.27E−76 |
| imm_16_49348200 | 16 | 50790699.00 | A | G | 0.180272168 | 0.009816232 | 2.52E−75 |
| rs9673419 | 16 | 50661273.00 | C | A | 0.206149785 | 0.011267574 | 8.93E−75 |
| imm_16_49400014 | 16 | 50842513.00 | A | T | −0.197300063 | 0.01081255 | 2.17E−74 |
| imm_16_49361788 | 16 | 50804287.00 | A | G | 0.178815574 | 0.009839662 | 8.45E−74 |
| imm_16_49338053 | 16 | 50780552.00 | A | G | −0.188471247 | 0.010402336 | 2.29E−73 |
| imm_16_49366227 | 16 | 50808726.00 | G | A | 0.175059722 | 0.009839162 | 8.13E−71 |
| imm_16_49397149 | 16 | 50839648.00 | A | G | 0.171975789 | 0.009896608 | 1.23E−67 |
| rs1109863 | 16 | 50692364.00 | G | A | 0.187527918 | 0.010830543 | 3.64E−67 |
| imm_16_49325723 | 16 | 50768222.00 | A | G | 0.168747415 | 0.009848582 | 8.25E−66 |
| imm_16_49325148 | 16 | 50767647.00 | A | G | 0.168891575 | 0.009871633 | 1.28E−65 |
| ccc0501502093290A0G | 5 | 150229136.00 | G | A | 0.291125596 | 0.017306877 | 1.70E−63 |
| imm_5_150226732 | 5 | 150246539.00 | G | C | 0.283065834 | 0.016852222 | 2.57E−63 |
| imm_5_150238675 | 5 | 150258482.00 | G | A | 0.284701447 | 0.017076097 | 2.08E−62 |
| imm_2_233902126 | 2 | 234237387.00 | A | G | −0.164608881 | 0.009874804 | 2.18E−62 |
| imm_16_49344648 | 16 | 50787147.00 | G | A | 0.162278915 | 0.009834695 | 3.63E−61 |
| imm_2_233892432 | 2 | 234227693.00 | G | A | −0.163298642 | 0.009902213 | 4.25E−61 |
| imm_16_49380465 | 16 | 50822964.00 | A | C | 0.161198504 | 0.009888505 | 9.61E−60 |
| imm_16_49377067 | 16 | 50819566.00 | G | A | 0.162285534 | 0.009965671 | 1.27E−59 |
| imm_16_49377411 | 16 | 50819910.00 | G | A | 0.159402951 | 0.009871419 | 1.17E−58 |
| imm_2_233833690 | 2 | 234168951.00 | A | G | −0.210867897 | 0.013085349 | 2.01E−58 |
| imm_2_233809681 | 2 | 234144942.00 | A | G | 0.173839675 | 0.010881844 | 1.90E−57 |
| imm_2_233899567 | NA | NA | NA | NA | −0.174583785 | 0.010992581 | 8.45E−57 |
| imm_2_233815784 | 2 | 234151045.00 | A | C | −0.236388874 | 0.014914648 | 1.42E−56 |
| imm_2_233907086 | 2 | 234242347.00 | G | A | −0.155881118 | 0.009885871 | 5.16E−56 |
| imm_2_233898230 | 2 | 234233491.00 | G | A | −0.160443565 | 0.010207918 | 1.15E−55 |
| imm_16_49399578 | 16 | 50842077.00 | A | G | 0.154225823 | 0.009860261 | 3.82E−55 |
| imm_2_233899497 | 2 | 234234758.00 | G | A | −0.15963384 | 0.010206564 | 3.87E−55 |
| imm_16_49399698 | 16 | 50842197.00 | A | C | 0.154151195 | 0.009857407 | 4.00E−55 |
| imm_16_49403663 | 16 | 50846162.00 | A | C | −0.165713154 | 0.010659505 | 1.69E−54 |
| rs1990623 | 16 | 50565970.00 | G | A | 0.202135957 | 0.013136578 | 1.99E−53 |
| imm_5_150157290 | 5 | 150177097.00 | A | C | 0.221427508 | 0.014473379 | 7.77E−53 |
| imm_5_150225225 | 5 | 150245032.00 | G | A | 0.265026612 | 0.017391806 | 1.96E−52 |
| imm_16_49329618 | 16 | 50772117.00 | A | G | −0.161690971 | 0.01071962 | 2.07E−51 |
| imm_5_150225322 | 5 | 150245129.00 | A | G | 0.244608412 | 0.016399739 | 2.62E−50 |
| imm_5_150209259 | 5 | 150229066.00 | G | A | 0.245540592 | 0.016477063 | 3.20E−50 |
| imm_5_150225917 | 5 | 150245724.00 | G | A | 0.246379007 | 0.016534858 | 3.27E−50 |
| imm_5_150225452 | 5 | 150245259.00 | A | G | 0.24547666 | 0.016482121 | 3.63E−50 |
| imm_5_150164746 | 5 | 150184553.00 | G | A | 0.251112724 | 0.016871055 | 4.17E−50 |
| imm_5_150239113 | 5 | 150258920.00 | A | C | 0.245460698 | 0.016493933 | 4.32E−50 |
| imm_5_150226046 | 5 | 150245853.00 | A | G | 0.245166512 | 0.016482925 | 4.86E−50 |
| imm_5_150206779 | 5 | 150226586.00 | G | A | 0.244710355 | 0.016453652 | 4.96E−50 |
| imm_5_150204572 | 5 | 150224379.00 | A | G | 0.245045259 | 0.016478998 | 5.15E−50 |
| imm_5_150258539 | 5 | 150278346.00 | C | A | 0.252825375 | 0.017006385 | 5.44E−50 |
| imm_5_150155003 | 5 | 150174810.00 | G | A | 0.252322293 | 0.016976692 | 5.74E−50 |
| imm_5_150208511 | 5 | 150228318.00 | A | G | 0.244930853 | 0.016480309 | 5.81E−50 |
| imm_5_150239835 | 5 | 150259642.00 | G | A | 0.245066905 | 0.01649914 | 6.62E−50 |
| imm_5_150206832 | 5 | 150226639.00 | G | A | 0.244664832 | 0.016476983 | 7.07E−50 |
| imm_5_150207092 | 5 | 150226899.00 | G | A | 0.244575333 | 0.016475468 | 7.52E−50 |
| imm_5_150237584 | 5 | 150257391.00 | A | G | 0.244595125 | 0.016482498 | 8.11E−50 |
| imm_5_150241500 | 5 | 150261307.00 | A | G | 0.24469205 | 0.016498664 | 9.23E−50 |
| imm_5_150225903 | 5 | 150245710.00 | A | G | 0.244251972 | 0.016471717 | 9.58E−50 |
| imm_5_150169480 | 5 | 150189287.00 | A | G | 0.250179022 | 0.016872779 | 9.75E−50 |
| imm_5_150166118 | 5 | 150185925.00 | C | A | 0.250192276 | 0.016875686 | 1.00E−49 |
| imm_5_150250244 | 5 | 150270051.00 | G | A | 0.244983439 | 0.016527487 | 1.04E−49 |
| imm_5_150250613 | 5 | 150270420.00 | G | A | 0.24491206 | 0.01652388 | 1.06E−49 |
| imm_5_150252410 | 5 | 150272217.00 | C | A | 0.245465066 | 0.016563076 | 1.09E−49 |
| imm_5_150208191 | 5 | 150227998.00 | A | G | 0.244230928 | 0.016484006 | 1.15E−49 |
| imm_5_150242335 | 5 | 150262142.00 | G | A | 0.244392381 | 0.016497641 | 1.19E−49 |
| imm_5_150220269 | 5 | 150240076.00 | A | G | 0.249707155 | 0.016856995 | 1.20E−49 |
| imm_2_233831759 | 2 | 234167020.00 | A | G | −0.157349388 | 0.010623164 | 1.23E−49 |
| imm_5_150154488 | 5 | 150174295.00 | C | A | 0.250807499 | 0.016934576 | 1.26E−49 |
| imm_5_150258102 | 5 | 150277909.00 | G | A | 0.251723092 | 0.017002492 | 1.36E−49 |
| imm_5_150246279 | 5 | 150266086.00 | G | A | 0.244139891 | 0.016491329 | 1.38E−49 |
| imm_5_150241557 | 5 | 150261364.00 | G | A | 0.249843141 | 0.016888136 | 1.60E−49 |
| imm_5_150238300 | 5 | 150258107.00 | G | A | 0.244055725 | 0.016498857 | 1.64E−49 |
| imm_5_150203915 | 5 | 150223722.00 | A | G | 0.244683754 | 0.016542524 | 1.67E−49 |
| imm_5_150253046 | 5 | 150272853.00 | G | A | 0.245358509 | 0.016589364 | 1.70E−49 |
| imm_5_150251978 | 5 | 150271785.00 | G | A | 0.243713736 | 0.016485219 | 1.86E−49 |
| imm_5_150211143 | 5 | 150230950.00 | A | G | 0.249157423 | 0.016862478 | 2.10E−49 |
| imm_5_150224466 | 5 | 150244273.00 | A | C | 0.243351211 | 0.016478935 | 2.38E−49 |
| imm_5_150250485 | 5 | 150270292.00 | G | A | 0.243570258 | 0.016500682 | 2.61E−49 |
| imm_5_150203930 | 5 | 150223737.00 | A | G | 0.243286355 | 0.016483874 | 2.69E−49 |
| imm_2_233838009 | 2 | 234173270.00 | C | G | −0.156706099 | 0.010619182 | 2.78E−49 |
| imm_12_39078464 | 12 | 40792197.00 | C | G | 0.446308691 | 0.030250389 | 2.91E−49 |
| imm_5_150155152 | 5 | 150174959.00 | A | G | 0.250290722 | 0.016973729 | 3.28E−49 |
| imm_5_150259411 | 5 | 150279218.00 | G | A | 0.253788138 | 0.01721171 | 3.31E−49 |
| imm_5_150219664 | 5 | 150239471.00 | A | C | 0.248585898 | 0.016868266 | 3.74E−49 |
| imm_2_233831061 | NA | NA | NA | NA | −0.16809129 | 0.011418698 | 4.75E−49 |
| imm_5_150166927 | 5 | 150186734.00 | C | A | 0.248250264 | 0.016870259 | 5.14E−49 |
| imm_5_150241098 | 5 | 150260905.00 | A | G | 0.242816654 | 0.016506622 | 5.54E−49 |
| imm_5_150231573 | 5 | 150251380.00 | G | A | 0.248169793 | 0.016878893 | 6.17E−49 |
| imm_5_150150076 | 5 | 150169883.00 | T | A | 0.252939601 | 0.017217248 | 7.36E−49 |
| imm_2_233809622 | 2 | 234144883.00 | G | A | −0.159568087 | 0.010861746 | 7.38E−49 |
| imm_5_150249525 | 5 | 150269332.00 | G | A | 0.241905016 | 0.016481291 | 8.98E−49 |
| imm_5_150237809 | 5 | 150257616.00 | G | A | 0.242465229 | 0.016524619 | 9.60E−49 |
| imm_5_150154751 | 5 | 150174558.00 | A | T | 0.249691231 | 0.017021791 | 1.02E−48 |
| imm_5_150240025 | 5 | 150259832.00 | A | G | 0.245043031 | 0.016733715 | 1.48E−48 |
| imm_5_150151752 | 5 | 150171559.00 | G | A | 0.24882095 | 0.017007825 | 1.81E−48 |
| imm_5_150150036 | 5 | 150169843.00 | A | C | 0.25192752 | 0.017220968 | 1.83E−48 |
| imm_5_150248802 | 5 | 150268609.00 | G | A | 0.241754066 | 0.016526478 | 1.85E−48 |
| imm_12_39100082 | 12 | 40813815.00 | T | A | 0.441444829 | 0.03037132 | 7.28E−48 |
| imm_2_233812782 | 2 | 234148043.00 | G | A | −0.159330352 | 0.010971358 | 8.74E−48 |
| imm_12_39094143 | 12 | 40807876.00 | C | A | 0.439383084 | 0.030418423 | 2.71E−47 |
| imm_12_39089461 | 12 | 40803194.00 | G | A | 0.43890233 | 0.030459088 | 4.51E−47 |
| imm_5_150303621 | 5 | 150323428.00 | G | A | 0.23612414 | 0.01653398 | 2.87E−46 |
| imm_5_150304815 | 5 | 150324622.00 | G | A | 0.233754238 | 0.016483158 | 1.19E−45 |
| imm_2_233835136 | 2 | 234170397.00 | G | A | −0.155415525 | 0.011003153 | 2.68E−45 |
| imm_16_49317481 | 16 | 50759980.00 | A | C | −0.154787225 | 0.011022416 | 8.51E−45 |
| imm_16_49308676 | 16 | 50751175.00 | A | C | −0.149378045 | 0.010660076 | 1.30E−44 |
| imm_5_150286725 | 5 | 150306532.00 | G | C | 0.233957116 | 0.016696593 | 1.31E−44 |
| imm_5_150275076 | 5 | 150294883.00 | G | A | 0.233919093 | 0.016697219 | 1.36E−44 |
| imm_5_150292065 | 5 | 150311872.00 | G | A | 0.233666641 | 0.016713498 | 2.04E−44 |
| imm_5_150280612 | 5 | 150300419.00 | A | G | 0.233902008 | 0.016743617 | 2.39E−44 |
| imm_2_233914124 | 2 | 234249385.00 | G | A | −0.1375258 | 0.009860318 | 3.26E−44 |
| imm_2_233911003 | 2 | 234246264.00 | G | A | −0.137123338 | 0.009856013 | 5.31E−44 |
| imm_2_233897461 | 2 | 234232722.00 | A | C | −0.146966885 | 0.010578657 | 7.01E−44 |
| imm_2_233864200 | 2 | 234199461.00 | G | A | −0.14306605 | 0.010310315 | 8.85E−44 |
| imm_5_150280471 | NA | NA | NA | NA | 0.253248882 | 0.018279944 | 1.20E−43 |
| imm_5_150252645 | 5 | 150272452.00 | G | A | 0.233777935 | 0.017051895 | 8.87E−43 |
| imm_5_150239060 | 5 | 150258867.00 | G | A | 0.232107486 | 0.016947814 | 1.08E−42 |
| imm_5_150252814 | 5 | 150272621.00 | A | C | 0.230880185 | 0.016861738 | 1.12E−42 |
| imm_2_233807983 | 2 | 234143244.00 | A | G | −0.144662453 | 0.010575392 | 1.35E−42 |
| imm_2_233828903 | NA | NA | NA | NA | −0.217203631 | 0.015884297 | 1.45E−42 |
| imm_5_150203708 | 5 | 150223515.00 | A | T | 0.231546701 | 0.016942737 | 1.61E−42 |
| imm_5_150151159 | 5 | 150170966.00 | A | G | 0.238782868 | 0.017476425 | 1.69E−42 |
| imm_12_38909591 | 12 | 40623324.00 | C | A | 0.44643886 | 0.032697344 | 1.92E−42 |
| imm_5_150152585 | NA | NA | NA | NA | 0.210789314 | 0.015484456 | 3.35E−42 |
| imm_2_233829896 | 2 | 234165157.00 | T | A | −0.202025357 | 0.014884212 | 5.78E−42 |
| imm_5_150309059 | 5 | 150328866.00 | G | A | 0.232352145 | 0.017123206 | 6.08E−42 |
| imm_5_150162903 | 5 | 150182710.00 | A | G | 0.242175266 | 0.017888968 | 9.37E−42 |
| imm_12_39027140 | 12 | 40740873.00 | G | A | 0.399930091 | 0.030119129 | 3.09E−40 |
| imm_2_233875711 | 2 | 234210972.00 | G | A | −0.133514336 | 0.010079249 | 4.73E−40 |
| imm_5_150155085 | 5 | 150174892.00 | C | A | 0.188289841 | 0.014215864 | 4.82E−40 |
| imm_12_39036462 | 12 | 40750195.00 | C | A | 0.399294182 | 0.030173119 | 5.63E−40 |
| imm_12_39037641 | 12 | 40751374.00 | A | G | 0.398939173 | 0.030151988 | 5.82E−40 |
| imm_12_38893976 | 12 | 40607709.00 | G | A | 0.438656236 | 0.033234199 | 8.90E−40 |
| imm_12_39017280 | 12 | 40731013.00 | G | A | 0.397503385 | 0.030141854 | 1.03E−39 |
| imm_12_39061394 | 12 | 40775127.00 | C | G | 0.386376197 | 0.02933239 | 1.27E−39 |
| imm_5_150213363 | 5 | 150233170.00 | G | A | 0.18530248 | 0.014071089 | 1.32E−39 |
| imm_5_150168265 | 5 | 150188072.00 | A | G | 0.182403254 | 0.013855369 | 1.40E−39 |
| imm_5_150209994 | 5 | 150229801.00 | C | A | 0.184485035 | 0.014016599 | 1.45E−39 |
| imm_12_39087563 | 12 | 40801296.00 | G | A | 0.41719381 | 0.031701857 | 1.49E−39 |
| imm_5_150310824 | 5 | 150330631.00 | A | G | 0.228576009 | 0.017371912 | 1.54E−39 |
| imm_5_150312674 | 5 | 150332481.00 | A | G | 0.228105145 | 0.017346377 | 1.70E−39 |
| imm_12_39055172 | 12 | 40768905.00 | G | A | 0.38550114 | 0.029323747 | 1.79E−39 |
| imm_12_39080166 | 12 | 40793899.00 | G | A | 0.386986999 | 0.029439201 | 1.81E−39 |
| imm_5_150224764 | 5 | 150244571.00 | A | G | 0.184786497 | 0.014058758 | 1.85E−39 |
| imm_12_39084680 | 12 | 40798413.00 | A | G | 0.386909768 | 0.029443571 | 1.92E−39 |
| imm_12_39082553 | 12 | 40796286.00 | G | A | 0.387253164 | 0.029476916 | 2.01E−39 |
| imm_12_39059207 | 12 | 40772940.00 | A | C | 0.38535556 | 0.029336217 | 2.05E−39 |
| imm_12_39079727 | 12 | 40793460.00 | A | G | 0.387383315 | 0.029499573 | 2.16E−39 |
| imm_12_39078567 | 12 | 40792300.00 | A | G | 0.389141609 | 0.029634732 | 2.18E−39 |
| imm_5_150219780 | 5 | 150239587.00 | A | G | 0.184449199 | 0.014054174 | 2.39E−39 |
| imm_5_150165294 | 5 | 150185101.00 | G | A | 0.181623395 | 0.013853186 | 2.86E−39 |
| imm_5_150224234 | 5 | 150244041.00 | A | C | 0.184248204 | 0.014058021 | 3.03E−39 |
| imm_12_39098525 | 12 | 40812258.00 | A | T | 0.38724281 | 0.029553302 | 3.16E−39 |
| imm_12_39078119 | 12 | 40791852.00 | G | A | 0.385285763 | 0.02940908 | 3.25E−39 |
| imm_12_39070841 | 12 | 40784574.00 | G | A | 0.384804407 | 0.029386437 | 3.53E−39 |
| imm_12_39078197 | 12 | 40791930.00 | A | G | 0.385249296 | 0.029428207 | 3.70E−39 |
| imm_12_39041556 | 12 | 40755289.00 | G | A | 0.395276414 | 0.030197902 | 3.78E−39 |
| imm_5_150154715 | 5 | 150174522.00 | G | C | 0.186909853 | 0.014280407 | 3.83E−39 |
| imm_12_39083849 | 12 | 40797582.00 | C | A | 0.385797484 | 0.029478874 | 3.89E−39 |
| imm_5_150165151 | 5 | 150184958.00 | A | G | 0.181271222 | 0.013853209 | 4.00E−39 |
| imm_12_39082632 | 12 | 40796365.00 | G | A | 0.385778857 | 0.029485407 | 4.08E−39 |
| imm_5_150165638 | 5 | 150185445.00 | A | G | 0.181179282 | 0.013848048 | 4.10E−39 |
| imm_16_49255572 | 16 | 50698071.00 | C | G | 0.176447108 | 0.013490938 | 4.34E−39 |
| imm_5_150164764 | 5 | 150184571.00 | G | A | 0.181213436 | 0.013856611 | 4.41E−39 |
| imm_12_39079123 | 12 | 40792856.00 | C | A | 0.384871558 | 0.029443571 | 4.79E−39 |
| imm_5_150315475 | 5 | 150335282.00 | A | G | 0.230700532 | 0.017649496 | 4.81E−39 |
| imm_12_39106670 | 12 | 40820403.00 | A | G | 0.387518432 | 0.02965007 | 4.90E−39 |
| imm_12_39083305 | 12 | 40797038.00 | G | A | 0.384872718 | 0.029466365 | 5.47E−39 |
| imm_5_150229730 | 5 | 150249537.00 | G | A | 0.18360432 | 0.014060896 | 5.74E−39 |
| imm_12_39112589 | 12 | 40826322.00 | A | G | 0.388093484 | 0.0297441 | 6.55E−39 |
| imm_12_39026953 | 12 | 40740686.00 | G | A | 0.410643264 | 0.031488745 | 7.16E−39 |
| imm_12_39078970 | 12 | 40792703.00 | A | G | 0.383389056 | 0.02943723 | 8.94E−39 |
| imm_12_39084129 | 12 | 40797862.00 | A | C | 0.384422048 | 0.029517689 | 9.00E−39 |
| imm_12_39113348 | 12 | 40827081.00 | G | A | 0.385739556 | 0.029650469 | 1.08E−38 |
| imm_12_39111215 | 12 | 40824948.00 | G | A | 0.385828408 | 0.029663931 | 1.08E−38 |
| imm_12_39111982 | 12 | 40825715.00 | A | C | 0.386146601 | 0.029700952 | 1.21E−38 |
| imm_5_150167998 | 5 | 150187805.00 | C | A | 0.179884816 | 0.013848437 | 1.40E−38 |
| imm_12_39112468 | 12 | 40826201.00 | A | G | 0.379480355 | 0.029222559 | 1.47E−38 |
| imm_12_39089396 | 12 | 40803129.00 | A | G | 0.384011778 | 0.029581627 | 1.56E−38 |
| imm_12_39114286 | 12 | 40828019.00 | G | A | 0.384814744 | 0.029652155 | 1.64E−38 |
| imm_12_39105991 | 12 | 40819724.00 | G | A | 0.384766076 | 0.029657344 | 1.72E−38 |
| imm_5_150233297 | 5 | 150253104.00 | A | G | 0.183358645 | 0.014135659 | 1.78E−38 |
| imm_12_39111484 | 12 | 40825217.00 | G | A | 0.385466822 | 0.029727733 | 1.89E−38 |
| imm_12_39113485 | 12 | 40827218.00 | A | C | 0.383815043 | 0.029609128 | 1.99E−38 |
| imm_12_39106268 | 12 | 40820001.00 | G | A | 0.384746828 | 0.029686293 | 2.05E−38 |
| imm_12_39109741 | 12 | 40823474.00 | A | G | 0.384898129 | 0.029703168 | 2.11E−38 |
| imm_12_39083472 | 12 | 40797205.00 | G | A | 0.382402475 | 0.029511488 | 2.12E−38 |
| imm_12_39079782 | 12 | 40793515.00 | A | G | 0.380764875 | 0.029389663 | 2.18E−38 |
| imm_12_39114089 | 12 | 40827822.00 | C | A | 0.384119095 | 0.029652487 | 2.23E−38 |
| imm_12_39111375 | 12 | 40825108.00 | G | A | 0.38448284 | 0.029681059 | 2.24E−38 |
| imm_12_39101827 | 12 | 40815560.00 | A | G | 0.382239667 | 0.029515179 | 2.33E−38 |
| imm_12_39104262 | 12 | 40817995.00 | C | A | 0.383786885 | 0.02963743 | 2.37E−38 |
| imm_5_150252255 | 5 | 150272062.00 | G | A | 0.223558182 | 0.017271717 | 2.55E−38 |
| imm_12_39099208 | 12 | 40812941.00 | G | A | 0.383340215 | 0.029629797 | 2.76E−38 |
| imm_12_39111967 | 12 | 40825700.00 | G | A | 0.384126111 | 0.02970331 | 2.97E−38 |
| imm_12_39096017 | 12 | 40809750.00 | G | A | 0.382438026 | 0.029591069 | 3.29E−38 |
| imm_12_39104435 | 12 | 40818168.00 | A | G | 0.382733076 | 0.029619173 | 3.39E−38 |
| imm_12_39095066 | 12 | 40808799.00 | A | G | 0.382267823 | 0.029588092 | 3.49E−38 |
| imm_12_39046898 | 12 | 40760631.00 | G | A | 0.385220224 | 0.029839904 | 3.98E−38 |
| imm_12_39100851 | 12 | 40814584.00 | A | G | 0.383108627 | 0.029681279 | 4.09E−38 |
| imm_12_39109497 | 12 | 40823230.00 | G | A | 0.383665609 | 0.029735544 | 4.35E−38 |
| imm_12_39100464 | 12 | 40814197.00 | A | G | 0.381594849 | 0.029576542 | 4.39E−38 |
| imm_12_39108613 | 12 | 40822346.00 | G | A | 0.383815681 | 0.029749531 | 4.41E−38 |
| imm_5_150162627 | 5 | 150182434.00 | G | A | 0.179214791 | 0.013892329 | 4.49E−38 |
| imm_12_39092861 | 12 | 40806594.00 | G | A | 0.381551686 | 0.029586193 | 4.72E−38 |
| imm_12_39091944 | 12 | 40805677.00 | A | G | 0.381714669 | 0.02960115 | 4.79E−38 |
| imm_12_39099163 | 12 | 40812896.00 | G | A | 0.382794536 | 0.029685504 | 4.80E−38 |
| imm_12_39099993 | 12 | 40813726.00 | A | G | 0.381941432 | 0.029621295 | 4.86E−38 |
| imm_12_39109269 | 12 | 40823002.00 | A | G | 0.384297374 | 0.029840739 | 5.97E−38 |
| imm_12_39100534 | 12 | 40814267.00 | G | A | 0.383859677 | 0.02982889 | 6.75E−38 |
| imm_12_39106216 | 12 | 40819949.00 | A | G | 0.380684954 | 0.029588463 | 6.99E−38 |
| imm_12_39090980 | 12 | 40804713.00 | A | G | 0.380352971 | 0.029571617 | 7.36E−38 |
| imm_12_39093178 | 12 | 40806911.00 | G | A | 0.380622327 | 0.029594258 | 7.43E−38 |
| imm_12_39098577 | 12 | 40812310.00 | A | G | 0.380878472 | 0.029617653 | 7.57E−38 |
| imm_12_39090800 | 12 | 40804533.00 | A | G | 0.380390688 | 0.029580032 | 7.59E−38 |
| imm_12_39108138 | 12 | 40821871.00 | A | C | 0.381199082 | 0.029652646 | 8.01E−38 |
| imm_12_39091414 | 12 | 40805147.00 | G | A | 0.380278973 | 0.02958704 | 8.29E−38 |
| imm_12_39109485 | 12 | 40823218.00 | A | G | 0.382271308 | 0.029750903 | 8.71E−38 |
| imm_12_39096428 | 12 | 40810161.00 | A | G | 0.381197769 | 0.029667862 | 8.73E−38 |
| imm_12_39098659 | 12 | 40812392.00 | A | G | 0.380953511 | 0.029660071 | 9.30E−38 |
| imm_12_39087797 | 12 | 40801530.00 | A | G | 0.380472039 | 0.029629919 | 9.69E−38 |
| imm_12_39099716 | 12 | 40813449.00 | C | A | 0.380428705 | 0.029631272 | 9.95E−38 |
| imm_12_39098686 | 12 | 40812419.00 | C | A | 0.380812357 | 0.029667442 | 1.03E−37 |
| imm_12_39093396 | 12 | 40807129.00 | A | G | 0.380551153 | 0.029681275 | 1.25E−37 |
| imm_12_39094939 | 12 | 40808672.00 | A | G | 0.378144023 | 0.02955516 | 1.76E−37 |
| imm_12_39098415 | 12 | 40812148.00 | A | G | 0.379094829 | 0.029629515 | 1.76E−37 |
| imm_12_39040575 | 12 | 40754308.00 | G | A | 0.378893316 | 0.02963113 | 1.94E−37 |
| imm_5_150203580 | 5 | 150223387.00 | G | A | 0.223100621 | 0.0174641 | 2.27E−37 |
| imm_12_39112288 | 12 | 40826021.00 | A | C | 0.378793768 | 0.02965177 | 2.27E−37 |
| imm_12_39098130 | 12 | 40811863.00 | A | C | 0.379471344 | 0.029706945 | 2.30E−37 |
| imm_12_39090360 | 12 | 40804093.00 | G | A | 0.378405107 | 0.029626992 | 2.34E−37 |
| imm_12_39093364 | 12 | 40807097.00 | A | G | 0.378671063 | 0.029674547 | 2.71E−37 |
| imm_12_39104678 | 12 | 40818411.00 | A | G | 0.38426436 | 0.030126012 | 2.91E−37 |
| imm_12_38933912 | 12 | 40647645.00 | A | C | 0.383192092 | 0.030043673 | 2.94E−37 |
| imm_12_39094197 | 12 | 40807930.00 | A | G | 0.378408814 | 0.029691446 | 3.34E−37 |
| imm_12_39007426 | 12 | 40721159.00 | G | A | 0.399519662 | 0.031350686 | 3.39E−37 |
| imm_12_39013412 | 12 | 40727145.00 | A | G | 0.386230496 | 0.03032033 | 3.62E−37 |
| imm_12_38936984 | 12 | 40650717.00 | A | G | 0.378981352 | 0.029756446 | 3.73E−37 |
| imm_12_39101528 | 12 | 40815261.00 | G | A | 0.378921042 | 0.029761264 | 3.93E−37 |
| imm_12_39091934 | 12 | 40805667.00 | A | C | 0.376405742 | 0.029613621 | 5.17E−37 |
| imm_12_39107745 | 12 | 40821478.00 | G | A | 0.374313324 | 0.029467996 | 5.74E−37 |
| imm_12_39110421 | 12 | 40824154.00 | A | G | 0.381407932 | 0.030075975 | 7.49E−37 |
| imm_12_38941140 | 12 | 40654873.00 | G | A | 0.377390673 | 0.029818773 | 1.03E−36 |
| imm_12_39100374 | 12 | 40814107.00 | G | A | 0.373635831 | 0.029538417 | 1.13E−36 |
| imm_12_39099837 | 12 | 40813570.00 | A | G | 0.373520393 | 0.029557657 | 1.32E−36 |
| imm_5_150150802 | 5 | 150170609.00 | A | G | 0.184305015 | 0.01461975 | 1.94E−36 |
| imm_12_39105263 | NA | NA | NA | NA | 0.404524175 | 0.032094664 | 2.00E−36 |
| imm_12_38921086 | 12 | 40634819.00 | A | G | 0.379285062 | 0.030112175 | 2.23E−36 |
| imm_12_38921258 | 12 | 40634991.00 | A | G | 0.378934323 | 0.030091673 | 2.32E−36 |
| imm_12_38938239 | 12 | 40651972.00 | A | C | 0.374511849 | 0.029865186 | 4.51E−36 |
| imm_5_150267038 | 5 | 150286845.00 | A | G | 0.17689627 | 0.014118641 | 5.16E−36 |
| imm_12_39077191 | 12 | 40790924.00 | A | G | 0.37626938 | 0.03014581 | 9.40E−36 |
| imm_12_38953004 | 12 | 40666737.00 | A | G | 0.372544822 | 0.029853267 | 9.70E−36 |
| imm_12_38963866 | 12 | 40677599.00 | G | A | 0.371967917 | 0.029827271 | 1.08E−35 |
| imm_12_38956323 | 12 | 40670056.00 | G | A | 0.371826931 | 0.029845137 | 1.26E−35 |
| imm_12_38963922 | 12 | 40677655.00 | A | G | 0.371493956 | 0.029828416 | 1.32E−35 |
| imm_12_38953930 | 12 | 40667663.00 | A | G | 0.371530105 | 0.029838144 | 1.37E−35 |
| imm_12_38997714 | 12 | 40711447.00 | A | C | 0.37006133 | 0.029742024 | 1.54E−35 |
| imm_12_39001350 | 12 | 40715083.00 | A | G | 0.369883703 | 0.029742024 | 1.66E−35 |
| imm_12_38994589 | 12 | 40708322.00 | A | G | 0.38263494 | 0.030812952 | 2.09E−35 |
| imm_12_39093004 | 12 | 40806737.00 | A | G | 0.364209279 | 0.029346482 | 2.29E−35 |
| imm_2_233875787 | 2 | 234211048.00 | G | A | −0.132728889 | 0.010729628 | 3.78E−35 |
| imm_12_39002701 | 12 | 40716434.00 | G | A | 0.367984739 | 0.029750564 | 3.85E−35 |
| imm_2_233916061 | 2 | 234251322.00 | A | C | −0.122189519 | 0.009907408 | 6.01E−35 |
| imm_12_38932004 | 12 | 40645737.00 | A | G | 0.377728067 | 0.03063141 | 6.14E−35 |
| imm_2_233908061 | 2 | 234243322.00 | A | G | −0.129471806 | 0.010539686 | 1.10E−34 |
| imm_16_49291534 | 16 | 50734033.00 | G | A | −0.132902085 | 0.010873631 | 2.36E−34 |
| imm_12_38822848 | 12 | 40536581.00 | G | A | 0.377487491 | 0.031074943 | 5.90E−34 |
| imm_12_39112802 | 12 | 40826535.00 | G | A | 0.379147203 | 0.031220772 | 6.17E−34 |
| imm_12_38916014 | 12 | 40629747.00 | A | G | 0.378055465 | 0.031176478 | 7.66E−34 |
| imm_16_49296606 | 16 | 50739105.00 | G | A | −0.131240194 | 0.010841568 | 9.90E−34 |
| imm_12_38855065 | 12 | 40568798.00 | G | A | 0.375613042 | 0.031218845 | 2.42E−33 |
| imm_12_39079244 | 12 | 40792977.00 | A | C | 0.360049738 | 0.029979293 | 3.15E−33 |
| imm_16_49291470 | 16 | 50733969.00 | A | G | −0.130714714 | 0.010901795 | 4.00E−33 |
| imm_12_39109264 | NA | NA | NA | NA | 0.392449448 | 0.032775022 | 4.86E−33 |
| imm_16_49295481 | 16 | 50737980.00 | A | G | −0.129958126 | 0.010862718 | 5.51E−33 |
| imm_12_38878895 | 12 | 40592628.00 | G | A | 0.378847312 | 0.031726897 | 7.24E−33 |
| imm_2_233910964 | 2 | 234246225.00 | A | G | −0.126148899 | 0.010564801 | 7.28E−33 |
| imm_2_233912663 | 2 | 234247924.00 | G | A | −0.125963338 | 0.010556575 | 8.04E−33 |
| imm_2_233912366 | 2 | 234247627.00 | A | G | −0.125854557 | 0.010560684 | 9.62E−33 |
| imm_2_233827154 | 2 | 234162415.00 | A | G | −0.131916823 | 0.011076605 | 1.06E−32 |
| imm_2_233846332 | 2 | 234181593.00 | G | C | −0.131319242 | 0.011123167 | 3.64E−32 |
| imm_12_39107545 | 12 | 40821278.00 | C | G | 0.364274002 | 0.031032858 | 8.11E−32 |
| imm_2_233851473 | 2 | 234186734.00 | A | C | −0.135548062 | 0.011566633 | 1.02E−31 |
| imm_12_39094260 | 12 | 40807993.00 | G | A | 0.355053311 | 0.030366227 | 1.39E−31 |
| imm_12_39077674 | 12 | 40791407.00 | A | G | 0.342907581 | 0.029402489 | 1.98E−31 |
| imm_2_233851228 | 2 | 234186489.00 | A | G | −0.129904699 | 0.011189769 | 3.70E−31 |
| rs6500315 | 16 | 50508101.00 | A | G | −0.139876648 | 0.012066385 | 4.51E−31 |
| imm_12_38776732 | 12 | 40490465.00 | C | A | 0.533145893 | 0.046106618 | 6.32E−31 |
| imm_12_39088649 | 12 | 40802382.00 | C | A | 0.358874296 | 0.031039555 | 6.43E−31 |
| imm_12_39085544 | 12 | 40799277.00 | A | C | 0.348389556 | 0.030135149 | 6.50E−31 |
| imm_16_49317232 | 16 | 50759731.00 | T | A | −0.378110432 | 0.032828913 | 1.08E−30 |
| imm_2_233911109 | 2 | 234246370.00 | A | G | −0.114443819 | 0.00993685 | 1.08E−30 |
| imm_5_150258780 | 5 | 150278587.00 | A | G | 0.165424259 | 0.014600569 | 9.32E−30 |
| imm_12_38877879 | 12 | 40591612.00 | A | C | 0.374859022 | 0.033846429 | 1.65E−28 |
| imm_2_233829231 | 2 | 234164492.00 | A | G | −0.240357308 | 0.022127197 | 1.74E−27 |
| imm_2_233830746 | 2 | 234166007.00 | G | A | −0.240235683 | 0.022124165 | 1.82E−27 |
| imm_12_38944422 | 12 | 40658155.00 | A | G | 0.34092985 | 0.0314867 | 2.54E−27 |
| imm_2_233909057 | 2 | 234244318.00 | G | A | −0.107999016 | 0.010000211 | 3.45E−27 |
| imm_12_39095605 | 12 | 40809338.00 | A | G | 0.334419888 | 0.031080936 | 5.34E−27 |
| imm_2_233850498 | 2 | 234185759.00 | A | G | −0.237223353 | 0.022112467 | 7.52E−27 |
| imm_2_233858402 | 2 | 234193663.00 | T | A | −0.237509905 | 0.022168193 | 8.75E−27 |
| imm_2_233836198 | 2 | 234171459.00 | A | G | −0.236975851 | 0.022134066 | 9.50E−27 |
| imm_2_233817248 | 2 | 234152509.00 | A | G | −0.233985421 | 0.021897696 | 1.19E−26 |
| imm_2_233822511 | 2 | 234157772.00 | A | G | −0.235225089 | 0.022106917 | 1.93E−26 |
| imm_2_233868852 | 2 | 234204113.00 | A | G | −0.117830969 | 0.011152275 | 4.30E−26 |
| imm_2_233875420 | 2 | 234210681.00 | A | G | −0.249237255 | 0.023758691 | 9.57E−26 |
| imm_2_233867060 | 2 | 234202321.00 | G | A | −0.113109276 | 0.010801104 | 1.16E−25 |
| imm_2_233875068 | 2 | 234210329.00 | A | G | −0.24763671 | 0.023788326 | 2.23E−25 |
| imm_2_233864276 | 2 | 234199537.00 | C | A | −0.112379041 | 0.010796501 | 2.26E−25 |
| imm_2_233872567 | 2 | 234207828.00 | G | A | −0.112426287 | 0.010820348 | 2.75E−25 |
| imm_2_233869338 | 2 | 234204599.00 | A | G | −0.111410917 | 0.010806854 | 6.40E−25 |
| imm_2_233869266 | 2 | 234204527.00 | A | G | −0.110646802 | 0.01080988 | 1.37E−24 |
| imm_12_39080560 | 12 | 40794293.00 | A | G | 0.407906302 | 0.040720971 | 1.28E−23 |
| imm_12_39042739 | 12 | 40756472.00 | C | A | 0.105510302 | 0.010543811 | 1.42E−23 |
| rs12149258 | 16 | 50536826.00 | G | A | −0.136655169 | 0.01372962 | 2.44E−23 |
| imm_12_39059018 | 12 | 40772751.00 | A | G | 0.401660317 | 0.040414091 | 2.83E−23 |
| imm_12_39044919 | 12 | 40758652.00 | A | G | 0.100936985 | 0.010277907 | 9.16E−23 |
| imm_2_233921844 | 2 | 234257105.00 | A | G | 0.096866471 | 0.009992969 | 3.21E−22 |
| imm_12_39046247 | 12 | 40759980.00 | A | G | 0.099552833 | 0.010275042 | 3.36E−22 |
| imm_12_39042954 | 12 | 40756687.00 | A | G | 0.099491458 | 0.010278565 | 3.68E−22 |
| imm_12_39047274 | 12 | 40761007.00 | A | G | 0.099412229 | 0.010279072 | 3.99E−22 |
| imm_2_233922792 | 2 | 234258053.00 | C | A | 0.100576309 | 0.010403774 | 4.15E−22 |
| imm_12_39048813 | 12 | 40762546.00 | A | G | 0.09890773 | 0.010271664 | 6.02E−22 |
| imm_12_38671385 | 12 | 40385118.00 | A | G | 0.243247485 | 0.025341437 | 8.09E−22 |
| imm_12_39048218 | 12 | 40761951.00 | G | A | 0.098536263 | 0.010274935 | 8.81E−22 |
| imm_16_49279294 | 16 | 50721793.00 | G | A | −0.107017507 | 0.011225878 | 1.53E−21 |
| imm_16_49271530 | 16 | 50714029.00 | G | A | −0.106623336 | 0.011187484 | 1.56E−21 |
| imm_2_233922191 | 2 | 234257452.00 | A | G | 0.094999414 | 0.009982046 | 1.78E−21 |
| imm_2_233923740 | 2 | 234259001.00 | A | C | 0.094755142 | 0.00997317 | 2.08E−21 |
| imm_2_233903639 | 2 | 234238900.00 | A | G | −0.105358377 | 0.01110401 | 2.35E−21 |
| imm_12_38699572 | 12 | 40413305.00 | A | C | 0.404936227 | 0.042690428 | 2.41E−21 |
| imm_12_38715963 | 12 | 40429696.00 | A | G | 0.404437752 | 0.04265065 | 2.48E−21 |
| imm_2_233902504 | 2 | 234237765.00 | A | C | −0.105294165 | 0.011108163 | 2.57E−21 |
| imm_2_233903523 | 2 | 234238784.00 | A | G | −0.10523335 | 0.01110401 | 2.62E−21 |
| imm_2_233922193 | 2 | 234257454.00 | G | A | 0.094089387 | 0.009948615 | 3.15E−21 |
| imm_2_233897974 | 2 | 234233235.00 | C | G | −0.104922767 | 0.011100615 | 3.32E−21 |
| imm_12_39043800 | 12 | 40757533.00 | A | G | 0.096843583 | 0.010247964 | 3.39E−21 |
| imm_2_233902993 | 2 | 234238254.00 | A | C | −0.104823714 | 0.011104003 | 3.72E−21 |
| imm_12_38767750 | 12 | 40481483.00 | A | G | 0.408238217 | 0.043253903 | 3.79E−21 |
| imm_12_38682716 | 12 | 40396449.00 | A | G | 0.403796812 | 0.042786042 | 3.81E−21 |
| imm_2_233899365 | 2 | 234234626.00 | A | G | −0.104645092 | 0.011097353 | 4.11E−21 |
| imm_5_150361647 | 5 | 150381454.00 | A | G | 0.177938966 | 0.018877132 | 4.25E−21 |
| imm_12_38766294 | 12 | 40480027.00 | A | C | 0.407382087 | 0.043253903 | 4.58E−21 |
| imm_12_38776917 | 12 | 40490650.00 | A | G | 0.407140745 | 0.043265789 | 4.95E−21 |
| imm_16_49271836 | 16 | 50714335.00 | G | A | −0.10559248 | 0.011264383 | 6.98E−21 |
| imm_16_49279543 | 16 | 50722042.00 | G | A | −0.105092867 | 0.011214471 | 7.17E−21 |
| rs128602 | 16 | 50999201.00 | G | A | 0.105659387 | 0.011281177 | 7.53E−21 |
| imm_12_38670265 | 12 | 40383998.00 | G | A | 0.232015876 | 0.024861645 | 1.04E−20 |
| imm_2_233922840 | 2 | 234258101.00 | A | G | 0.09305615 | 0.009972019 | 1.04E−20 |
| imm_12_38711871 | 12 | 40425604.00 | G | A | 0.399292972 | 0.042792004 | 1.05E−20 |
| imm_12_38896017 | 12 | 40609750.00 | G | A | 0.403650685 | 0.043366676 | 1.30E−20 |
| imm_12_38786462 | 12 | 40500195.00 | A | C | 0.402565838 | 0.043372342 | 1.67E−20 |
| imm_2_233905837 | 2 | 234241098.00 | A | G | 0.103317824 | 0.011139255 | 1.77E−20 |
| imm_12_38791734 | 12 | 40505467.00 | A | T | 0.401991564 | 0.04340975 | 2.04E−20 |
| imm_2_233892262 | 2 | 234227523.00 | A | G | 0.103331564 | 0.011171599 | 2.26E−20 |
| imm_2_233905423 | 2 | 234240684.00 | G | A | 0.102805225 | 0.0111379 | 2.70E−20 |
| imm_16_49314777 | 16 | 50757276.00 | A | G | 0.159111504 | 0.017250849 | 2.88E−20 |
| imm_12_38904364 | 12 | 40618097.00 | G | A | 0.400477315 | 0.043435781 | 2.97E−20 |
| imm_12_38669409 | 12 | 40383142.00 | A | G | 0.228984201 | 0.024910643 | 3.85E−20 |
| imm_12_38787389 | 12 | 40501122.00 | A | G | 0.398432618 | 0.043401519 | 4.30E−20 |
| imm_16_49271834 | 16 | 50714333.00 | C | G | 0.105583765 | 0.011560157 | 6.64E−20 |
| imm_12_38817958 | 12 | 40531691.00 | G | A | 0.395469301 | 0.04338003 | 7.77E−20 |
| imm_16_49275542 | 16 | 50718041.00 | G | A | 0.10234856 | 0.011230881 | 8.00E−20 |
| imm_16_49273899 | 16 | 50716398.00 | A | C | 0.10269197 | 0.011268861 | 8.02E−20 |
| imm_12_38792849 | 12 | 40506582.00 | G | A | 0.387472215 | 0.042643149 | 1.02E−19 |
| imm_16_49282290 | 16 | 50724789.00 | G | A | 0.102418806 | 0.011284091 | 1.12E−19 |
| imm_12_38657778 | 12 | 40371511.00 | A | C | 0.226368727 | 0.025029431 | 1.51E−19 |
| imm_12_38900701 | 12 | 40614434.00 | A | G | 0.124692308 | 0.013823667 | 1.88E−19 |
| imm_12_38658001 | 12 | 40371734.00 | A | C | 0.22542621 | 0.025046454 | 2.25E−19 |
| imm_12_38829030 | 12 | 40542763.00 | G | A | 0.3897868 | 0.043342087 | 2.40E−19 |
| imm_16_49277175 | 16 | 50719674.00 | A | G | 0.101403112 | 0.011288966 | 2.65E−19 |
| imm_2_233899595 | 2 | 234234856.00 | A | G | 0.198134331 | 0.022065849 | 2.73E−19 |
| imm_12_38814699 | 12 | 40528432.00 | G | A | 0.391373421 | 0.043586626 | 2.73E−19 |
| imm_12_38888207 | 12 | 40601940.00 | A | G | 0.385958987 | 0.043047955 | 3.08E−19 |
| rs6500336 | 16 | 50865549.00 | C | A | 0.133049272 | 0.014924154 | 4.88E−19 |
| imm_2_233921588 | 2 | 234256849.00 | A | G | 0.088369483 | 0.009922632 | 5.30E−19 |
| imm_12_38885665 | 12 | 40599398.00 | G | A | 0.383663529 | 0.043166189 | 6.22E−19 |
| imm_12_38685416 | 12 | 40399149.00 | G | A | 0.391938619 | 0.044149823 | 6.84E−19 |
| imm_2_233921265 | 2 | 234256526.00 | A | G | 0.087923476 | 0.009916016 | 7.53E−19 |
| imm_16_49272480 | 16 | 50714979.00 | A | G | 0.100211756 | 0.011302863 | 7.58E−19 |
| imm_2_233918336 | 2 | 234253597.00 | C | A | 0.087434977 | 0.009914956 | 1.16E−18 |
| imm_12_39104527 | 12 | 40818260.00 | A | C | 0.088970932 | 0.010100226 | 1.26E−18 |
| imm_16_49282115 | 16 | 50724614.00 | G | A | 0.099252326 | 0.011324735 | 1.88E−18 |
| imm_12_39106475 | 12 | 40820208.00 | A | G | 0.087298423 | 0.009998722 | 2.53E−18 |
| imm_12_39095099 | 12 | 40808832.00 | G | A | 0.087888371 | 0.010097186 | 3.20E−18 |
| imm_16_49399660 | 16 | 50842159.00 | G | A | 0.16535233 | 0.019025816 | 3.59E−18 |
| imm_12_38872374 | 12 | 40586107.00 | A | G | 0.38548377 | 0.044437371 | 4.14E−18 |
| imm_12_38873788 | 12 | 40587521.00 | G | A | 0.37434824 | 0.043222964 | 4.68E−18 |
| imm_16_49280872 | 16 | 50723371.00 | A | T | 0.098130401 | 0.01133704 | 4.90E−18 |
| imm_16_49280575 | 16 | 50723074.00 | G | A | 0.097362158 | 0.011316844 | 7.75E−18 |
| imm_12_39070775 | 12 | 40784508.00 | A | G | 0.088542584 | 0.010363587 | 1.30E−17 |
| imm_12_39072369 | 12 | 40786102.00 | G | A | 0.088663781 | 0.010386163 | 1.38E−17 |
| imm_16_49282363 | 16 | 50724862.00 | C | A | −0.096477793 | 0.011319088 | 1.55E−17 |
| imm_12_39072482 | 12 | 40786215.00 | A | G | 0.0882464 | 0.010359812 | 1.62E−17 |
| imm_16_49385102 | 16 | 50827601.00 | A | G | −0.11276466 | 0.013265108 | 1.88E−17 |
| imm_12_39072370 | 12 | 40786103.00 | A | G | 0.087985485 | 0.010386163 | 2.42E−17 |
| imm_5_150038606 | 5 | 150058413.00 | G | A | 0.138172906 | 0.016363499 | 3.07E−17 |
| imm_16_49281588 | 16 | 50724087.00 | G | C | −0.095694004 | 0.011354376 | 3.52E−17 |
| imm_12_39071800 | 12 | 40785533.00 | A | G | 0.086860719 | 0.010362883 | 5.21E−17 |
| imm_12_39073652 | 12 | 40787385.00 | C | A | 0.086719742 | 0.010366431 | 5.99E−17 |
| imm_2_233937223 | 2 | 234272484.00 | A | G | 0.093982495 | 0.011264387 | 7.22E−17 |
| imm_12_39072986 | 12 | 40786719.00 | A | G | 0.086129211 | 0.010385935 | 1.11E−16 |
| imm_12_39071775 | 12 | 40785508.00 | G | A | 0.085863191 | 0.010359797 | 1.15E−16 |
| imm_12_39083040 | 12 | 40796773.00 | G | C | −0.084145857 | 0.010171543 | 1.31E−16 |
| imm_2_233910123 | 2 | 234245384.00 | A | G | −0.180142422 | 0.0217872 | 1.36E−16 |
| imm_2_233811578 | NA | NA | NA | NA | −0.180552693 | 0.021862353 | 1.47E−16 |
| imm_12_39092494 | 12 | 40806227.00 | A | G | −0.083957961 | 0.010170515 | 1.52E−16 |
| imm_2_233899661 | 2 | 234234922.00 | A | G | −0.179854234 | 0.021793043 | 1.55E−16 |
| imm_2_233900193 | 2 | 234235454.00 | G | A | −0.178939155 | 0.021733413 | 1.82E−16 |
| imm_16_49278418 | 16 | 50720917.00 | G | A | −0.095672538 | 0.011631578 | 1.95E−16 |
| imm_12_39083115 | 12 | 40796848.00 | T | A | −0.083232491 | 0.010171616 | 2.77E−16 |
| imm_12_39106899 | NA | NA | NA | NA | −0.08756646 | 0.010782395 | 4.61E−16 |
| imm_12_39076229 | 12 | 40789962.00 | A | G | 0.345835019 | 0.042857642 | 7.07E−16 |
| imm_16_49404333 | 16 | 50846832.00 | G | A | −0.096955549 | 0.012040189 | 8.10E−16 |
| imm_2_233848477 | 2 | 234183738.00 | G | A | −0.161664323 | 0.020202437 | 1.22E−15 |
| ccc0501502612490G0A | 5 | 150281056.00 | A | G | 0.471446703 | 0.058925751 | 1.24E−15 |
| imm_16_49402777 | 16 | 50845276.00 | A | G | 0.149650788 | 0.018710881 | 1.26E−15 |
| rs1477172 | 16 | 50881186.00 | A | C | −0.114769021 | 0.014356724 | 1.31E−15 |
| imm_2_233834041 | 2 | 234169302.00 | A | G | 0.100541908 | 0.012589812 | 1.39E−15 |
| imm_12_39044558 | 12 | 40758291.00 | A | G | 0.093890351 | 0.011765774 | 1.46E−15 |
| imm_2_233860812 | 2 | 234196073.00 | G | A | −0.162750905 | 0.020413143 | 1.55E−15 |
| imm_2_233814123 | 2 | 234149384.00 | A | C | −0.163155171 | 0.020480705 | 1.64E−15 |
| imm_2_233854098 | 2 | 234189359.00 | G | A | −0.184396328 | 0.023147392 | 1.64E−15 |
| imm_12_39080260 | 12 | 40793993.00 | A | G | −0.082011082 | 0.01029597 | 1.65E−15 |
| imm_2_233868282 | 2 | 234203543.00 | G | A | −0.17020461 | 0.021449473 | 2.10E−15 |
| imm_2_233868479 | 2 | 234203740.00 | G | A | −0.170842663 | 0.021569057 | 2.36E−15 |
| imm_2_233868615 | 2 | 234203876.00 | A | G | −0.171446958 | 0.021661575 | 2.48E−15 |
| imm_2_233864992 | 2 | 234200253.00 | G | A | −0.161984087 | 0.020470362 | 2.51E−15 |
| imm_2_233866506 | 2 | 234201767.00 | G | A | −0.160282469 | 0.020307206 | 2.95E−15 |
| imm_2_233845718 | 2 | 234180979.00 | A | G | −0.171671027 | 0.021764027 | 3.07E−15 |
| imm_2_233846295 | 2 | 234181556.00 | A | C | −0.171781778 | 0.02178076 | 3.10E−15 |
| imm_2_233872221 | 2 | 234207482.00 | A | T | −0.170672663 | 0.02170553 | 3.75E−15 |
| imm_2_233834784 | 2 | 234170045.00 | A | G | −0.160057583 | 0.020361105 | 3.81E−15 |
| imm_2_233840728 | 2 | 234175989.00 | G | A | −0.160038658 | 0.02037593 | 4.02E−15 |
| imm_16_49401763 | 16 | 50844262.00 | G | A | 0.145556725 | 0.018587786 | 4.85E−15 |
| imm_2_234051542 | 2 | 234386803.00 | C | A | 0.084408509 | 0.010795484 | 5.33E−15 |
| imm_16_49402160 | 16 | 50844659.00 | A | G | 0.145067056 | 0.01860406 | 6.31E−15 |
| imm_16_49400987 | 16 | 50843486.00 | G | A | 0.144796789 | 0.018572425 | 6.37E−15 |
| imm_16_49401923 | 16 | 50844422.00 | C | A | 0.144477686 | 0.018600614 | 8.01E−15 |
| imm_2_233866929 | 2 | 234202190.00 | G | A | −0.182745964 | 0.023614495 | 1.00E−14 |
| imm_2_233820501 | 2 | 234155762.00 | C | A | −0.157076641 | 0.020299977 | 1.01E−14 |
| imm_2_233822899 | 2 | 234158160.00 | A | G | −0.157260549 | 0.020346871 | 1.08E−14 |
| rs10094579 | 8 | 90849305.00 | A | C | −0.103915422 | 0.013474435 | 1.24E−14 |
| imm_2_233809819 | 2 | 234145080.00 | G | A | −0.157635398 | 0.020515101 | 1.54E−14 |
| imm_5_150342312 | 5 | 150362119.00 | C | G | 0.113780833 | 0.014830139 | 1.69E−14 |
| imm_2_233871306 | 2 | 234206567.00 | A | G | −0.180046114 | 0.023497699 | 1.83E−14 |
| imm_2_233817912 | 2 | 234153173.00 | A | G | −0.156432903 | 0.020530359 | 2.54E−14 |
| imm_2_233866396 | 2 | 234201657.00 | G | A | −0.17959324 | 0.023581024 | 2.62E−14 |
| imm_2_233872216 | 2 | 234207477.00 | G | A | −0.179594533 | 0.023590998 | 2.68E−14 |
| imm_2_233865738 | 2 | 234200999.00 | A | T | −0.178907605 | 0.023640135 | 3.79E−14 |
| imm_16_49397860 | 16 | 50840359.00 | A | G | 0.138780645 | 0.018378993 | 4.32E−14 |
| imm_12_38851428 | 12 | 40565161.00 | A | C | 0.129621062 | 0.01720415 | 4.91E−14 |
| imm_6_91032711 | 6 | 90975990.00 | A | G | −0.079696106 | 0.010604484 | 5.68E−14 |
| imm_6_91040756 | 6 | 90984035.00 | A | G | −0.077964896 | 0.010396661 | 6.43E−14 |
| imm_2_233850278 | 2 | 234185539.00 | A | G | −0.175579129 | 0.023425094 | 6.61E−14 |
| imm_2_233867505 | 2 | 234202766.00 | A | G | −0.178094409 | 0.023777524 | 6.89E−14 |
| imm_16_49400910 | 16 | 50843409.00 | G | A | 0.138820377 | 0.018534271 | 6.89E−14 |
| imm_6_91043280 | 6 | 90986559.00 | G | A | −0.077689602 | 0.010394707 | 7.78E−14 |
| imm_2_233894219 | 2 | 234229480.00 | A | G | −0.168247178 | 0.022539509 | 8.36E−14 |
| imm_2_233864140 | 2 | 234199401.00 | A | G | −0.175824521 | 0.023560952 | 8.49E−14 |
| imm_2_233861341 | 2 | 234196602.00 | A | G | −0.175080205 | 0.023523185 | 9.85E−14 |
| imm_6_91043407 | 6 | 90986686.00 | A | G | −0.077301125 | 0.010397021 | 1.05E−13 |
| imm_6_91043470 | 6 | 90986749.00 | G | A | −0.077191661 | 0.010392874 | 1.11E−13 |
| rs7015630 | 8 | 90875918.00 | G | A | −0.089250119 | 0.012027329 | 1.17E−13 |
| imm_6_91040571 | 6 | 90983850.00 | T | A | −0.076941169 | 0.010393128 | 1.33E−13 |
| imm_6_91038378 | 6 | 90981657.00 | A | G | −0.076930321 | 0.010397065 | 1.37E−13 |
| imm_6_91039108 | 6 | 90982387.00 | G | A | −0.076902854 | 0.010409863 | 1.50E−13 |
| imm_12_39086236 | NA | NA | NA | NA | −0.083023018 | 0.011251811 | 1.60E−13 |
| imm_12_38858065 | 12 | 40571798.00 | A | G | 0.36515723 | 0.04964652 | 1.91E−13 |
| imm_16_49284576 | 16 | 50727075.00 | A | G | −0.090256193 | 0.012289058 | 2.07E−13 |
| imm_6_91035104 | 6 | 90978383.00 | G | A | −0.076278259 | 0.010409966 | 2.35E−13 |
| imm_2_233899418 | 2 | 234234679.00 | G | A | −0.177196729 | 0.024186924 | 2.37E−13 |
| imm_6_91064748 | 6 | 91008027.00 | A | C | −0.076818178 | 0.01048964 | 2.42E−13 |
| imm_2_233961389 | 2 | 234296650.00 | A | G | −0.071657481 | 0.009825113 | 3.02E−13 |
| rs13262484 | 8 | 90774723.00 | G | A | −0.141561657 | 0.019441444 | 3.30E−13 |
| imm_2_233807358 | 2 | 234142619.00 | A | G | −0.197090572 | 0.027158213 | 3.95E−13 |
| imm_2_233888625 | 2 | 234223886.00 | A | G | −0.17203806 | 0.023777102 | 4.64E−13 |
| imm_6_91068394 | 6 | 91011673.00 | A | G | −0.0758213 | 0.010481821 | 4.70E−13 |
| rs28370650 | 12 | 40399948.00 | A | T | 0.35788359 | 0.049646016 | 5.65E−13 |
| imm_2_233901600 | 2 | 234236861.00 | G | A | −0.098434895 | 0.013668914 | 5.96E−13 |
| imm_2_233894059 | 2 | 234229320.00 | G | A | −0.170558352 | 0.023703399 | 6.22E−13 |
| imm_2_233889420 | 2 | 234224681.00 | A | G | −0.170452716 | 0.023706539 | 6.47E−13 |
| imm_2_233893685 | 2 | 234228946.00 | G | A | −0.169859377 | 0.023667676 | 7.13E−13 |
| imm_2_233879965 | 2 | 234215226.00 | A | C | −0.170831654 | 0.023860277 | 8.09E−13 |
| imm_12_38941974 | 12 | 40655707.00 | A | G | −0.144168041 | 0.020137918 | 8.12E−13 |
| imm_2_233892438 | 2 | 234227699.00 | A | G | −0.175086611 | 0.024493244 | 8.78E−13 |
| imm_2_233900041 | NA | NA | NA | NA | −0.182424473 | 0.025539435 | 9.14E−13 |
| imm_6_91062948 | 6 | 91006227.00 | A | G | −0.074932942 | 0.010494417 | 9.31E−13 |
| imm_6_91044233 | 6 | 90987512.00 | G | C | −0.078507998 | 0.011092174 | 1.46E−12 |
| imm_6_91043074 | 6 | 90986353.00 | G | A | −0.076657086 | 0.010858343 | 1.67E−12 |
| imm_2_233819008 | 2 | 234154269.00 | A | G | −0.186312086 | 0.026427237 | 1.79E−12 |
| imm_12_38943967 | 12 | 40657700.00 | G | C | −0.140991197 | 0.020066715 | 2.12E−12 |
| imm_12_38989038 | 12 | 40702771.00 | A | G | −0.14080219 | 0.020070158 | 2.29E−12 |
| imm_16_49259458 | 16 | 50701957.00 | A | G | −0.221933006 | 0.031643532 | 2.32E−12 |
| imm_2_233899170 | 2 | 234234431.00 | A | G | −0.169288087 | 0.024273686 | 3.08E−12 |
| imm_12_39052519 | 12 | 40766252.00 | G | A | −0.134451054 | 0.019348553 | 3.68E−12 |
| imm_16_49320272 | 16 | 50762771.00 | G | A | 0.226038595 | 0.032692635 | 4.71E−12 |
| imm_6_91034882 | 6 | 90978161.00 | A | G | −0.094115755 | 0.013623566 | 4.90E−12 |
| imm_12_38989254 | 12 | 40702987.00 | A | G | −0.138424531 | 0.020049438 | 5.05E−12 |
| imm_12_38972113 | 12 | 40685846.00 | A | G | −0.138850658 | 0.020113334 | 5.08E−12 |
| imm_16_49400857 | 16 | 50843356.00 | C | G | 0.13164186 | 0.019070743 | 5.10E−12 |
| imm_12_38977211 | 12 | 40690944.00 | A | G | −0.137996885 | 0.019995072 | 5.14E−12 |
| imm_6_138028843 | 6 | 137987150.00 | A | C | −0.09124935 | 0.013267667 | 6.09E−12 |
| imm_12_38963275 | 12 | 40677008.00 | A | G | −0.136971477 | 0.019921894 | 6.18E−12 |
| imm_12_38976249 | 12 | 40689982.00 | G | A | −0.137140138 | 0.019957226 | 6.34E−12 |
| imm_2_233903688 | 2 | 234238949.00 | A | G | −0.146188372 | 0.02128551 | 6.51E−12 |
| imm_12_38947548 | 12 | 40661281.00 | G | A | −0.136938196 | 0.01996183 | 6.89E−12 |
| rs7758080 | NA | NA | NA | NA | 0.080006919 | 0.011673614 | 7.20E−12 |
| imm_2_233980133 | NA | NA | NA | NA | −0.171264094 | 0.02502705 | 7.75E−12 |
| imm_2_233961183 | NA | NA | NA | NA | 0.073496898 | 0.010771037 | 8.88E−12 |
| imm_12_38963851 | 12 | 40677584.00 | A | C | −0.139526181 | 0.020452057 | 8.97E−12 |
| imm_12_38975188 | 12 | 40688921.00 | G | A | −0.136205764 | 0.01996916 | 9.05E−12 |
| rs2029923 | 16 | 50916464.00 | C | A | 0.071231755 | 0.01044895 | 9.29E−12 |
| imm_16_49297083 | 16 | 50739582.00 | C | G | 0.071208672 | 0.010447398 | 9.37E−12 |
| imm_12_39012195 | 12 | 40725928.00 | G | A | −0.131167462 | 0.019258089 | 9.69E−12 |
| imm_12_38979492 | 12 | 40693225.00 | C | A | −0.134360189 | 0.019735759 | 9.90E−12 |
| imm_12_38948306 | 12 | 40662039.00 | A | G | −0.136100844 | 0.019995548 | 1.00E−11 |
| imm_12_39024275 | 12 | 40738008.00 | A | G | −0.130918933 | 0.019261602 | 1.07E−11 |
| imm_12_39002741 | 12 | 40716474.00 | G | A | −0.136256857 | 0.0200593 | 1.10E−11 |
| imm_12_39042474 | 12 | 40756207.00 | G | A | −0.131090034 | 0.019303136 | 1.11E−11 |
| imm_5_150331722 | 5 | 150351529.00 | G | A | 0.096775126 | 0.014277673 | 1.22E−11 |
| imm_2_234045727 | 2 | 234380988.00 | A | C | 0.082633195 | 0.012191282 | 1.22E−11 |
| imm_12_39000168 | 12 | 40713901.00 | A | T | −0.074444992 | 0.01100205 | 1.32E−11 |
| imm_12_38973302 | 12 | 40687035.00 | A | G | −0.134462533 | 0.01996573 | 1.64E−11 |
| imm_16_49314041 | 16 | 50756540.00 | G | C | 0.211421604 | 0.031415468 | 1.70E−11 |
| imm_12_38967208 | 12 | 40680941.00 | G | A | −0.134334856 | 0.019995388 | 1.84E−11 |
| imm_12_39019167 | 12 | 40732900.00 | C | A | 0.065807941 | 0.009807341 | 1.95E−11 |
| imm_5_150331059 | 5 | 150350866.00 | A | G | 0.096938007 | 0.014471549 | 2.11E−11 |
| imm_5_150331183 | 5 | 150350990.00 | A | G | 0.09682709 | 0.014471549 | 2.22E−11 |
| imm_12_39024570 | 12 | 40738303.00 | G | A | 0.065557792 | 0.009810339 | 2.35E−11 |
| imm_12_39052867 | 12 | 40766600.00 | A | G | −0.127728232 | 0.019141432 | 2.51E−11 |
| imm_6_138064741 | 6 | 138023048.00 | A | G | −0.082164157 | 0.012345163 | 2.82E−11 |
| imm_2_234055353 | 2 | 234390614.00 | A | G | 0.081920391 | 0.012328547 | 3.04E−11 |
| imm_12_39017873 | 12 | 40731606.00 | G | A | 0.065343155 | 0.009837397 | 3.09E−11 |
| imm_16_49395448 | 16 | 50837947.00 | A | G | 0.116852399 | 0.017592827 | 3.09E−11 |
| imm_6_138025162 | 6 | 137983469.00 | A | C | −0.076161445 | 0.011484073 | 3.31E−11 |
| imm_2_234036405 | 2 | 234371666.00 | A | C | 0.08024372 | 0.012102393 | 3.35E−11 |
| imm_5_150335147 | 5 | 150354954.00 | A | G | 0.095921323 | 0.014478777 | 3.47E−11 |
| imm_12_38819604 | 12 | 40533337.00 | C | A | 0.112363427 | 0.016962967 | 3.50E−11 |
| imm_6_138051552 | 6 | 138009859.00 | G | A | −0.076651674 | 0.011578619 | 3.59E−11 |
| imm_5_150338039 | 5 | 150357846.00 | A | C | 0.095411824 | 0.014478743 | 4.40E−11 |
| imm_5_150339598 | 5 | 150359405.00 | G | A | 0.095231769 | 0.014469691 | 4.66E−11 |
| imm_6_138028676 | 6 | 137986983.00 | A | G | −0.075600966 | 0.011488282 | 4.68E−11 |
| imm_16_49372933 | 16 | 50815432.00 | A | G | 0.111953121 | 0.017067949 | 5.41E−11 |
| imm_5_150344859 | 5 | 150364666.00 | G | A | 0.094854971 | 0.014466054 | 5.49E−11 |
| imm_6_138064497 | 6 | 138022804.00 | A | C | −0.0755475 | 0.011551198 | 6.14E−11 |
| imm_16_49382332 | 16 | 50824831.00 | G | A | 0.115088315 | 0.017601627 | 6.21E−11 |
| imm_2_234043825 | 2 | 234379086.00 | A | G | 0.079219531 | 0.012128998 | 6.52E−11 |
| imm_2_233854145 | 2 | 234189406.00 | A | G | −0.152462109 | 0.023346492 | 6.56E−11 |
| imm_6_138003826 | 6 | 137962133.00 | A | G | −0.075109146 | 0.011531703 | 7.35E−11 |
| imm_16_49371498 | 16 | 50813997.00 | A | G | 0.11128325 | 0.017087961 | 7.40E−11 |
| imm_16_49392481 | 16 | 50834980.00 | A | G | 0.114265292 | 0.017562687 | 7.71E−11 |
| imm_6_138060132 | 6 | 138018439.00 | G | A | −0.074874644 | 0.011526405 | 8.25E−11 |
| imm_16_49382412 | 16 | 50824911.00 | A | T | 0.113971343 | 0.017556542 | 8.49E−11 |
| imm_12_38989178 | 12 | 40702911.00 | A | G | −0.128850155 | 0.019873999 | 8.97E−11 |
| imm_6_138061859 | 6 | 138020166.00 | C | A | −0.074750625 | 0.011530301 | 8.99E−11 |
| imm_5_150341913 | 5 | 150361720.00 | G | A | 0.094962335 | 0.014649786 | 9.04E−11 |
| imm_5_150334064 | 5 | 150353871.00 | G | C | 0.094045149 | 0.014510661 | 9.11E−11 |
| imm_5_150338544 | 5 | 150358351.00 | T | A | 0.093659475 | 0.014471461 | 9.67E−11 |
| imm_6_138011185 | 6 | 137969492.00 | G | A | −0.074804202 | 0.011559913 | 9.74E−11 |
| imm_16_49330670 | 16 | 50773169.00 | A | C | −0.191776278 | 0.029641012 | 9.80E−11 |
| imm_5_150337794 | 5 | 150357601.00 | G | A | 0.093445607 | 0.014443601 | 9.82E−11 |
| imm_12_38822956 | 12 | 40536689.00 | A | G | 0.297240277 | 0.04599879 | 1.03E−10 |
| imm_16_49385019 | 16 | 50827518.00 | A | G | 0.113415533 | 0.017558004 | 1.05E−10 |
| imm_2_234107739 | 2 | 234443000.00 | A | G | 0.078033172 | 0.012103795 | 1.14E−10 |
| imm_12_39017773 | 12 | 40731506.00 | G | C | 0.062928361 | 0.009836889 | 1.58E−10 |
| imm_16_49396798 | 16 | 50839297.00 | A | G | 0.111002176 | 0.017397415 | 1.77E−10 |
| imm_2_234116459 | 2 | 234451720.00 | G | A | 0.077190668 | 0.012103825 | 1.80E−10 |
| imm_12_38952187 | 12 | 40665920.00 | A | G | −0.140854382 | 0.022110595 | 1.88E−10 |
| imm_2_234103093 | 2 | 234438354.00 | G | A | 0.076758811 | 0.012103795 | 2.27E−10 |
| imm_12_39072764 | 12 | 40786497.00 | A | G | −0.123599838 | 0.019495749 | 2.30E−10 |
| imm_12_38858491 | 12 | 40572224.00 | A | G | 0.068830462 | 0.010887557 | 2.58E−10 |
| imm_2_234049335 | 2 | 234384596.00 | A | G | 0.076219928 | 0.012057136 | 2.59E−10 |
| imm_2_233964658 | 2 | 234299919.00 | G | A | 0.062102139 | 0.009864725 | 3.07E−10 |
| imm_2_234050015 | 2 | 234385276.00 | G | A | −0.159790302 | 0.025456612 | 3.45E−10 |
| imm_12_39092382 | 12 | 40806115.00 | A | T | −0.121814127 | 0.019436779 | 3.68E−10 |
| imm_2_234108061 | 2 | 234443322.00 | G | A | 0.075083997 | 0.01198632 | 3.75E−10 |
| imm_2_234111250 | 2 | 234446511.00 | G | A | 0.074954719 | 0.011988307 | 4.04E−10 |
| imm_2_234114201 | 2 | 234449462.00 | A | G | 0.074812491 | 0.011977125 | 4.20E−10 |
| imm_5_150349507 | 5 | 150369314.00 | A | G | 0.092447019 | 0.01480792 | 4.29E−10 |
| imm_2_233896140 | 2 | 234231401.00 | G | A | −0.204009773 | 0.032726732 | 4.55E−10 |
| imm_12_39094348 | 12 | 40808081.00 | A | G | −0.120990621 | 0.019417249 | 4.63E−10 |
| imm_12_39086534 | 12 | 40800267.00 | A | G | −0.121786464 | 0.019549296 | 4.67E−10 |
| imm_2_234118627 | 2 | 234453888.00 | G | A | 0.074793852 | 0.012015571 | 4.82E−10 |
| imm_2_234115561 | 2 | 234450822.00 | A | G | 0.074575752 | 0.011989227 | 4.96E−10 |
| imm_2_233936261 | 2 | 234271522.00 | A | G | −0.062554253 | 0.010058183 | 5.00E−10 |
| imm_2_234105256 | 2 | 234440517.00 | G | A | 0.074371645 | 0.011973229 | 5.25E−10 |
| imm_2_234059308 | NA | NA | NA | NA | 0.08005841 | 0.012895149 | 5.35E−10 |
| imm_2_234106556 | 2 | 234441817.00 | A | G | 0.074396233 | 0.011988318 | 5.44E−10 |
| imm_2_234114055 | 2 | 234449316.00 | G | A | 0.074337349 | 0.011988341 | 5.62E−10 |
| imm_2_233901427 | 2 | 234236688.00 | A | G | −0.156328664 | 0.025214461 | 5.65E−10 |
| imm_2_234105132 | 2 | 234440393.00 | A | G | 0.074404254 | 0.012020028 | 6.02E−10 |
| imm_2_234096756 | 2 | 234432017.00 | G | A | 0.074159417 | 0.011984929 | 6.10E−10 |
| imm_2_234116225 | 2 | 234451486.00 | G | A | 0.074384188 | 0.012035248 | 6.39E−10 |
| imm_2_234053428 | 2 | 234388689.00 | A | G | 0.074605702 | 0.012079969 | 6.58E−10 |
| imm_2_234103952 | 2 | 234439213.00 | A | G | 0.074095638 | 0.011999481 | 6.62E−10 |
| imm_2_234080405 | 2 | 234415666.00 | G | A | 0.073832528 | 0.011960355 | 6.70E−10 |
| imm_2_233933047 | 2 | 234268308.00 | C | A | −0.062108577 | 0.010073794 | 7.03E−10 |
| imm_2_234063323 | 2 | 234398584.00 | A | G | 0.073714514 | 0.011966045 | 7.26E−10 |
| imm_2_234071899 | 2 | 234407160.00 | A | G | 0.073729783 | 0.011970008 | 7.30E−10 |
| imm_2_234113682 | 2 | 234448943.00 | C | A | 0.073814052 | 0.011988352 | 7.41E−10 |
| imm_2_234099360 | 2 | 234434621.00 | A | G | 0.073630642 | 0.011961922 | 7.49E−10 |
| imm_2_234118133 | 2 | 234453394.00 | C | A | 0.073779791 | 0.011988541 | 7.55E−10 |
| imm_12_38874675 | 12 | 40588408.00 | A | G | 0.064384929 | 0.010462948 | 7.57E−10 |
| imm_2_234015993 | 2 | 234351254.00 | C | A | 0.172485199 | 0.028054439 | 7.84E−10 |
| imm_2_234086768 | 2 | 234422029.00 | G | A | 0.073310295 | 0.011927202 | 7.92E−10 |
| imm_2_234073638 | 2 | 234408899.00 | G | A | 0.073531982 | 0.011966105 | 8.00E−10 |
| imm_2_234050873 | 2 | 234386134.00 | G | A | 0.07325476 | 0.011924844 | 8.10E−10 |
| imm_2_234092357 | 2 | 234427618.00 | C | A | 0.073445233 | 0.01196148 | 8.24E−10 |
| imm_2_234114534 | 2 | 234449795.00 | A | C | 0.073714036 | 0.012008312 | 8.33E−10 |
| imm_2_234063156 | 2 | 234398417.00 | G | A | 0.073455931 | 0.011972912 | 8.51E−10 |
| imm_2_234101919 | 2 | 234437180.00 | A | G | 0.073537799 | 0.011988354 | 8.56E−10 |
| imm_2_234050757 | 2 | 234386018.00 | G | A | 0.073134922 | 0.011924838 | 8.62E−10 |
| imm_2_233973521 | 2 | 234308782.00 | A | C | 0.060611652 | 0.009895536 | 9.06E−10 |
| imm_2_234095434 | 2 | 234430695.00 | A | G | 0.073295981 | 0.011968836 | 9.13E−10 |
| imm_2_234100808 | 2 | 234436069.00 | A | G | −0.156129496 | 0.025504429 | 9.26E−10 |
| imm_12_38875374 | 12 | 40589107.00 | A | G | 0.06395744 | 0.010466544 | 9.92E−10 |
| imm_2_234080540 | 2 | 234415801.00 | G | A | 0.072744199 | 0.011927329 | 1.07E−09 |
| imm_2_233961895 | 2 | 234297156.00 | G | A | −0.06104824 | 0.010010329 | 1.07E−09 |
| imm_12_38877384 | 12 | 40591117.00 | G | A | 0.063820523 | 0.010469856 | 1.09E−09 |
| imm_2_234060393 | 2 | 234395654.00 | G | A | 0.072688767 | 0.011930393 | 1.11E−09 |
| imm_2_234087860 | 2 | 234423121.00 | G | A | 0.072841373 | 0.011964399 | 1.14E−09 |
| imm_2_234065106 | 2 | 234400367.00 | A | C | 0.072780598 | 0.011964265 | 1.18E−09 |
| imm_12_38874857 | 12 | 40588590.00 | A | G | 0.063646672 | 0.010466316 | 1.19E−09 |
| imm_2_234059068 | 2 | 234394329.00 | A | C | 0.072550702 | 0.011934354 | 1.21E−09 |
| imm_2_233813142 | 2 | 234148403.00 | A | G | 0.176887219 | 0.029108958 | 1.23E−09 |
| imm_2_233909134 | 2 | 234244395.00 | A | G | −0.144359675 | 0.023800849 | 1.32E−09 |
| imm_2_234096061 | 2 | 234431322.00 | G | C | 0.074811605 | 0.012337072 | 1.33E−09 |
| rs4785461 | 16 | 50875380.00 | C | A | −0.1271356 | 0.020980357 | 1.36E−09 |
| imm_12_38877440 | 12 | 40591173.00 | G | A | 0.063358235 | 0.010489244 | 1.54E−09 |
| imm_2_234079353 | 2 | 234414614.00 | G | A | 0.075564458 | 0.012518761 | 1.58E−09 |
| imm_2_234099578 | 2 | 234434839.00 | G | A | 0.072163557 | 0.011980043 | 1.71E−09 |
| imm_2_234052560 | 2 | 234387821.00 | G | A | 0.071952554 | 0.011950517 | 1.73E−09 |
| imm_5_150320875 | NA | NA | NA | NA | 0.097327553 | 0.016183364 | 1.81E−09 |
| imm_16_49402384 | 16 | 50844883.00 | A | G | −0.171391172 | 0.028499179 | 1.81E−09 |
| imm_12_38995680 | 12 | 40709413.00 | T | A | 0.061572783 | 0.010251566 | 1.90E−09 |
| imm_2_234061281 | 2 | 234396542.00 | A | G | 0.071609485 | 0.01194352 | 2.03E−09 |
| imm_2_234050448 | 2 | 234385709.00 | G | A | 0.074559074 | 0.012440745 | 2.06E−09 |
| imm_2_233956345 | 2 | 234291606.00 | A | G | −0.060147488 | 0.010051019 | 2.17E−09 |
| imm_12_38992798 | 12 | 40706531.00 | G | A | 0.061271317 | 0.010251566 | 2.28E−09 |
| imm_2_234107933 | 2 | 234443194.00 | A | C | 0.073456008 | 0.012294088 | 2.30E−09 |
| imm_2_233884691 | 2 | 234219952.00 | A | G | 0.163736397 | 0.027425018 | 2.37E−09 |
| imm_2_233957423 | 2 | 234292684.00 | A | G | −0.060112322 | 0.010075362 | 2.43E−09 |
| imm_2_234089762 | 2 | 234425023.00 | T | A | 0.071014834 | 0.01196485 | 2.93E−09 |
| imm_12_39111184 | 12 | 40824917.00 | C | A | 0.058875086 | 0.009933953 | 3.09E−09 |
| imm_12_38992642 | 12 | 40706375.00 | C | A | 0.060668387 | 0.010251566 | 3.26E−09 |
| imm_2_234044862 | 2 | 234380123.00 | G | A | 0.060644246 | 0.010284399 | 3.71E−09 |
| imm_12_39026632 | 12 | 40740365.00 | A | C | −0.062589045 | 0.010641745 | 4.07E−09 |
| imm_12_39108279 | 12 | 40822012.00 | G | A | 0.05842948 | 0.009943628 | 4.20E−09 |
| imm_2_233924209 | 2 | 234259470.00 | A | G | −0.059883397 | 0.010201329 | 4.35E−09 |
| imm_12_39012045 | 12 | 40725778.00 | G | A | 0.059984343 | 0.010246909 | 4.80E−09 |
| imm_12_39028521 | 12 | 40742254.00 | A | G | −0.062172446 | 0.010649029 | 5.27E−09 |
| imm_6_138127058 | 6 | 138085365.00 | C | A | −0.058155925 | 0.009961672 | 5.28E−09 |
| imm_6_138120478 | 6 | 138078785.00 | A | G | −0.057894428 | 0.009933936 | 5.61E−09 |
| imm_6_138123502 | 6 | 138081809.00 | A | G | −0.057149174 | 0.00981179 | 5.73E−09 |
| imm_6_138124455 | 6 | 138082762.00 | A | G | −0.06097005 | 0.010470811 | 5.78E−09 |
| imm_6_138118783 | 6 | 138077090.00 | A | G | −0.057016237 | 0.009803232 | 6.02E−09 |
| imm_6_138133834 | 6 | 138092141.00 | A | G | −0.057166399 | 0.009846755 | 6.41E−09 |
| imm_6_138126889 | 6 | 138085196.00 | A | G | −0.056854169 | 0.00980341 | 6.65E−09 |
| imm_6_138138055 | 6 | 138096362.00 | T | A | −0.058774405 | 0.010135793 | 6.68E−09 |
| imm_2_234112612 | 2 | 234447873.00 | C | A | 0.069590425 | 0.012006834 | 6.80E−09 |
| imm_6_138119016 | 6 | 138077323.00 | A | T | −0.056754994 | 0.009799527 | 6.97E−09 |
| imm_6_138127148 | 6 | 138085455.00 | G | A | −0.056863781 | 0.009835343 | 7.40E−09 |
| imm_6_138126941 | 6 | 138085248.00 | A | G | −0.056864842 | 0.009846538 | 7.69E−09 |
| imm_6_138120366 | 6 | 138078673.00 | C | A | −0.057202179 | 0.009913059 | 7.91E−09 |
| imm_2_233856884 | 2 | 234192145.00 | A | G | −0.187407353 | 0.032480552 | 7.94E−09 |
| imm_16_49317539 | 16 | 50760038.00 | A | G | −0.163611191 | 0.028369162 | 8.06E−09 |
| imm_6_138138080 | 6 | 138096387.00 | G | A | −0.056764901 | 0.009850355 | 8.28E−09 |
| imm_6_138131833 | 6 | 138090140.00 | A | G | −0.057375725 | 0.009961746 | 8.43E−09 |
| imm_12_39098390 | 12 | 40812123.00 | A | G | −0.060754511 | 0.010561602 | 8.80E−09 |
| imm_16_49403355 | 16 | 50845854.00 | G | A | −0.164815205 | 0.028692021 | 9.23E−09 |
| imm_6_138122241 | 6 | 138080548.00 | G | C | −0.056240044 | 0.009799859 | 9.53E−09 |
| imm_6_138127923 | 6 | 138086230.00 | A | G | −0.056492916 | 0.009849835 | 9.73E−09 |
| imm_12_39096860 | 12 | 40810593.00 | G | A | 0.057908393 | 0.010108508 | 1.01E−08 |
| imm_2_233869776 | 2 | 234205037.00 | G | A | −0.160510601 | 0.028028742 | 1.02E−08 |
| imm_12_38858414 | 12 | 40572147.00 | G | A | −0.056469396 | 0.009882965 | 1.10E−08 |
| imm_6_91070750 | 6 | 91014029.00 | T | A | −0.056756898 | 0.009964275 | 1.23E−08 |
| imm_12_38819618 | 12 | 40533351.00 | A | G | 0.059916279 | 0.010520246 | 1.23E−08 |
| imm_12_39000101 | 12 | 40713834.00 | C | A | 0.059548195 | 0.010457037 | 1.24E−08 |
| imm_2_233988971 | 2 | 234324232.00 | G | A | 0.056044895 | 0.009861113 | 1.32E−08 |
| imm_16_49383963 | 16 | 50826462.00 | A | C | 0.101512994 | 0.017896834 | 1.41E−08 |
| imm_6_138135010 | 6 | 138093317.00 | G | A | −0.055803766 | 0.009850505 | 1.47E−08 |
| imm_12_39101797 | 12 | 40815530.00 | A | C | −0.101690438 | 0.017956024 | 1.48E−08 |
| imm_6_138117846 | 6 | 138076153.00 | G | A | −0.055623058 | 0.0098264 | 1.51E−08 |
| imm_6_138112980 | 6 | 138071287.00 | A | G | −0.055305585 | 0.009822835 | 1.80E−08 |
| imm_12_38896246 | 12 | 40609979.00 | A | G | −0.055531761 | 0.009880259 | 1.90E−08 |
| rs2881698 | 7 | 6544691.00 | A | G | −0.068650982 | 0.012220185 | 1.93E−08 |
| imm_2_234104861 | NA | NA | NA | NA | 0.07480929 | 0.013317129 | 1.94E−08 |
| imm_6_138121923 | 6 | 138080230.00 | G | A | −0.055084042 | 0.00980954 | 1.96E−08 |
| imm_16_49348392 | 16 | 50790891.00 | G | A | −0.116267036 | 0.02071156 | 1.98E−08 |
| imm_12_38770252 | 12 | 40483985.00 | A | G | −0.057126359 | 0.010180402 | 2.01E−08 |
| imm_12_39011227 | 12 | 40724960.00 | A | C | −0.056344852 | 0.010056002 | 2.11E−08 |
| imm_5_150321182 | 5 | 150340989.00 | C | A | 0.119472917 | 0.021346096 | 2.18E−08 |
| imm_12_38896985 | 12 | 40610718.00 | A | G | −0.055116256 | 0.009879777 | 2.42E−08 |
| imm_12_38832768 | 12 | 40546501.00 | G | A | −0.056015222 | 0.01007308 | 2.68E−08 |
| imm_12_38895147 | 12 | 40608880.00 | A | G | −0.054899957 | 0.009880672 | 2.76E−08 |
| imm_16_49404218 | 16 | 50846717.00 | A | C | −0.138006877 | 0.024861989 | 2.84E−08 |
| imm_12_38949863 | 12 | 40663596.00 | A | C | −0.058865211 | 0.010607364 | 2.87E−08 |
| imm_12_38750970 | 12 | 40464703.00 | A | G | −0.056031368 | 0.010157141 | 3.46E−08 |
| imm_12_38989113 | 12 | 40702846.00 | A | T | 0.05761066 | 0.010448857 | 3.52E−08 |
| imm_12_38784593 | 12 | 40498326.00 | A | T | −0.05433401 | 0.009855847 | 3.53E−08 |
| imm_2_233996301 | 2 | 234331562.00 | G | A | −0.055775317 | 0.010118194 | 3.54E−08 |
| imm_16_49261528 | 16 | 50704027.00 | A | G | −0.247399292 | 0.044961085 | 3.74E−08 |
| imm_2_234068927 | 2 | 234404188.00 | A | C | 0.068434028 | 0.012473273 | 4.10E−08 |
| imm_12_39053956 | 12 | 40767689.00 | A | G | −0.057474618 | 0.010488218 | 4.26E−08 |
| imm_6_138135996 | 6 | 138094303.00 | A | G | −0.14319282 | 0.02618615 | 4.54E−08 |
| imm_12_38779683 | 12 | 40493416.00 | A | G | −0.055411809 | 0.010151686 | 4.80E−08 |
| imm_12_38752293 | 12 | 40466026.00 | C | A | −0.05533451 | 0.010155808 | 5.08E−08 |
| imm_5_150345215 | 5 | 150365022.00 | G | A | 0.081389935 | 0.014946421 | 5.17E−08 |
| imm_12_38664914 | 12 | 40378647.00 | A | G | 0.054357387 | 0.009986592 | 5.24E−08 |
| imm_2_233907889 | NA | NA | NA | NA | −0.087589045 | 0.01611554 | 5.48E−08 |
| imm_12_39090055 | 12 | 40803788.00 | A | G | 0.053741745 | 0.009893742 | 5.58E−08 |
| imm_6_138138991 | 6 | 138097298.00 | A | C | −0.05489362 | 0.010115371 | 5.74E−08 |
| imm_12_39061834 | 12 | 40775567.00 | A | G | −0.056786063 | 0.010481675 | 6.04E−08 |
| imm_12_39002282 | 12 | 40716015.00 | G | A | 0.058520696 | 0.01081035 | 6.18E−08 |
| imm_12_38768268 | 12 | 40482001.00 | G | A | −0.054491724 | 0.010068036 | 6.22E−08 |
| ccc0501501660190C0G | 5 | 150185826.00 | G | C | 0.371003672 | 0.068576889 | 6.30E−08 |
| imm_2_234088492 | 2 | 234423753.00 | A | G | 0.157555423 | 0.029146867 | 6.46E−08 |
| imm_2_234040916 | 2 | 234376177.00 | A | G | −0.053217264 | 0.009847417 | 6.51E−08 |
| imm_12_39051036 | 12 | 40764769.00 | C | A | −0.056571648 | 0.010475331 | 6.65E−08 |
| imm_2_234082303 | 2 | 234417564.00 | A | G | 0.150451855 | 0.027896507 | 6.92E−08 |
| imm_6_138117136 | 6 | 138075443.00 | A | G | −0.054240464 | 0.010069259 | 7.17E−08 |
| imm_16_49283737 | 16 | 50726236.00 | C | G | −0.353952881 | 0.065977282 | 8.11E−08 |
| imm_2_233854700 | 2 | 234189961.00 | G | A | 0.085206663 | 0.01590895 | 8.51E−08 |
| imm_12_38839552 | 12 | 40553285.00 | T | A | −0.053905863 | 0.010065848 | 8.54E−08 |
| imm_12_38835975 | 12 | 40549708.00 | G | A | −0.053128716 | 0.009930409 | 8.79E−08 |
| imm_12_38732017 | 12 | 40445750.00 | A | G | −0.053536654 | 0.010010244 | 8.88E−08 |
| imm_2_233937981 | 2 | 234273242.00 | G | A | 0.053910104 | 0.010083667 | 8.98E−08 |
| imm_12_38783362 | 12 | 40497095.00 | A | G | −0.053263876 | 0.009963668 | 9.00E−08 |
| imm_12_39112112 | 12 | 40825845.00 | G | A | −0.057605045 | 0.01077632 | 9.02E−08 |
| imm_12_38818418 | 12 | 40532151.00 | A | G | −0.052701004 | 0.00988207 | 9.66E−08 |
| imm_6_138064233 | 6 | 138022540.00 | C | A | −0.05379925 | 0.010091284 | 9.75E−08 |
| imm_12_38831363 | 12 | 40545096.00 | A | G | −0.05264624 | 0.00987991 | 9.90E−08 |
| imm_12_38772872 | 12 | 40486605.00 | G | A | −0.053492133 | 0.01003985 | 9.93E−08 |
| imm_6_138056604 | 6 | 138014911.00 | G | A | −0.053622278 | 0.010068263 | 1.00E−07 |
| imm_6_138075311 | 6 | 138033618.00 | A | G | −0.05484887 | 0.010343013 | 1.14E−07 |
| imm_6_138057818 | 6 | 138016125.00 | G | A | −0.053296771 | 0.010064836 | 1.19E−07 |
| imm_12_38835230 | 12 | 40548963.00 | C | A | −0.052117049 | 0.009882089 | 1.34E−07 |
| imm_12_38859741 | 12 | 40573474.00 | A | C | −0.053743957 | 0.010192829 | 1.34E−07 |
| imm_12_38693380 | 12 | 40407113.00 | A | G | −0.056556161 | 0.010728898 | 1.35E−07 |
| imm_5_150381489 | 5 | 150401296.00 | G | A | 0.091539857 | 0.017366097 | 1.36E−07 |
| imm_12_38810346 | 12 | 40524079.00 | G | A | −0.051940366 | 0.009862541 | 1.39E−07 |
| imm_2_233986491 | 2 | 234321752.00 | G | A | −0.053074447 | 0.010089786 | 1.44E−07 |
| imm_6_138077896 | 6 | 138036203.00 | G | A | −0.054407752 | 0.010344028 | 1.44E−07 |
| imm_2_233988303 | 2 | 234323564.00 | A | G | −0.053060776 | 0.010097575 | 1.48E−07 |
| imm_6_138075987 | 6 | 138034294.00 | G | A | −0.054297099 | 0.010340553 | 1.51E−07 |
| imm_6_138094373 | 6 | 138052680.00 | A | G | −0.052815721 | 0.010058976 | 1.52E−07 |
| imm_12_39108363 | 12 | 40822096.00 | A | G | −0.074039821 | 0.014109612 | 1.54E−07 |
| imm_12_38838469 | 12 | 40552202.00 | G | C | −0.051826325 | 0.009878734 | 1.55E−07 |
| imm_12_38846470 | 12 | 40560203.00 | G | A | −0.051827054 | 0.009883723 | 1.57E−07 |
| rs17227589 | 16 | 50973123.00 | A | G | 0.061231072 | 0.011700191 | 1.66E−07 |
| imm_16_49368474 | 16 | 50810973.00 | C | A | −0.182952669 | 0.034967065 | 1.68E−07 |
| imm_2_233988000 | 2 | 234323261.00 | G | A | −0.052778297 | 0.010093785 | 1.71E−07 |
| imm_12_38853117 | 12 | 40566850.00 | G | A | −0.051663405 | 0.00988192 | 1.71E−07 |
| imm_16_49317048 | 16 | 50759547.00 | T | A | −0.055503223 | 0.010625161 | 1.75E−07 |
| imm_12_38811859 | 12 | 40525592.00 | G | A | −0.051387208 | 0.009852569 | 1.83E−07 |
| imm_6_91044593 | 6 | 90987872.00 | G | A | −0.05156813 | 0.009904023 | 1.92E−07 |
| imm_12_38750217 | 12 | 40463950.00 | G | A | −0.052276259 | 0.010040358 | 1.92E−07 |
| imm_12_39072547 | 12 | 40786280.00 | A | C | −0.055932919 | 0.01074318 | 1.93E−07 |
| imm_12_38848280 | 12 | 40562013.00 | A | G | −0.051436219 | 0.009887128 | 1.97E−07 |
| imm_12_38849173 | 12 | 40562906.00 | A | G | −0.051397993 | 0.009882705 | 1.98E−07 |
| imm_12_38919581 | 12 | 40633314.00 | G | A | −0.051818916 | 0.009965518 | 1.99E−07 |
| imm_12_38808601 | 12 | 40522334.00 | G | A | −0.051188124 | 0.009852498 | 2.04E−07 |
| imm_12_38811027 | 12 | 40524760.00 | A | G | −0.051149001 | 0.009852429 | 2.09E−07 |
| imm_2_233817861 | 2 | 234153122.00 | A | G | 0.083726447 | 0.016132868 | 2.11E−07 |
| imm_12_39015922 | NA | NA | NA | NA | −0.059809637 | 0.011527816 | 2.12E−07 |
| imm_12_38811586 | 12 | 40525319.00 | A | G | −0.051093536 | 0.009852637 | 2.15E−07 |
| imm_12_38813662 | 12 | 40527395.00 | A | G | −0.051105469 | 0.00988092 | 2.31E−07 |
| imm_2_233980102 | 2 | 234315363.00 | A | G | −0.052189475 | 0.010094336 | 2.34E−07 |
| imm_5_150287162 | 5 | 150306969.00 | A | G | −0.052427951 | 0.010141195 | 2.34E−07 |
| imm_6_138101381 | 6 | 138059688.00 | G | A | −0.051891656 | 0.010045271 | 2.39E−07 |
| imm_12_38947670 | 12 | 40661403.00 | A | T | 0.054042786 | 0.010480498 | 2.52E−07 |
| imm_2_233994288 | 2 | 234329549.00 | A | G | −0.051946432 | 0.01009446 | 2.66E−07 |
| imm_12_38851141 | 12 | 40564874.00 | G | A | −0.050749767 | 0.009885899 | 2.84E−07 |
| imm_12_38801003 | 12 | 40514736.00 | G | A | −0.050526592 | 0.009850352 | 2.91E−07 |
| imm_6_137997482 | 6 | 137955789.00 | G | A | −0.054933073 | 0.01071264 | 2.93E−07 |
| imm_6_138109756 | 6 | 138068063.00 | A | G | −0.051565501 | 0.01006805 | 3.03E−07 |
| ccc0501502715600T0C | 5 | 150291367.00 | A | G | −0.050660567 | 0.009915757 | 3.24E−07 |
| imm_2_233942377 | 2 | 234277638.00 | A | G | −0.133155866 | 0.026167665 | 3.61E−07 |
| imm_16_49384443 | 16 | 50826942.00 | A | G | −0.17672942 | 0.034939527 | 4.23E−07 |
| imm_6_137989681 | 6 | 137947988.00 | A | G | −0.05391726 | 0.010669317 | 4.34E−07 |
| imm_5_150296798 | 5 | 150316605.00 | A | C | −0.051326612 | 0.010157688 | 4.35E−07 |
| imm_6_137982920 | 6 | 137941227.00 | G | A | −0.053988277 | 0.010692828 | 4.44E−07 |
| imm_2_234021546 | 2 | 234356807.00 | A | G | 0.143979313 | 0.028520817 | 4.46E−07 |
| imm_12_38800919 | 12 | 40514652.00 | A | G | −0.049672913 | 0.009850446 | 4.59E−07 |
| rs6570957 | 6 | 149585510.00 | A | C | 0.050034635 | 0.009922854 | 4.60E−07 |
| imm_12_38789300 | 12 | 40503033.00 | T | A | −0.04957913 | 0.009836897 | 4.65E−07 |
| imm_12_38897885 | 12 | 40611618.00 | A | C | −0.12160266 | 0.024137369 | 4.71E−07 |
| imm_6_137993864 | 6 | 137952171.00 | A | G | −0.053952221 | 0.01072058 | 4.84E−07 |
| imm_6_91076025 | 6 | 91019304.00 | C | A | −0.05049206 | 0.010036178 | 4.88E−07 |
| rs1001965 | 6 | 149587810.00 | A | G | 0.049953412 | 0.009946061 | 5.10E−07 |
| imm_2_234074540 | 2 | 234409801.00 | G | A | 0.162144303 | 0.032302514 | 5.18E−07 |
| imm_6_138094739 | NA | NA | NA | NA | −0.055451981 | 0.011062877 | 5.37E−07 |
| imm_5_150295407 | 5 | 150315214.00 | G | A | 0.072811698 | 0.014551235 | 5.62E−07 |
| imm_12_38806763 | 12 | 40520496.00 | A | G | −0.049371441 | 0.009874949 | 5.74E−07 |
| imm_12_38768161 | 12 | 40481894.00 | A | G | −0.050873693 | 0.010188066 | 5.93E−07 |
| imm_6_91053070 | NA | NA | NA | NA | −0.052815369 | 0.010645407 | 7.00E−07 |
| imm_5_150415673 | 5 | 150435480.00 | G | C | −0.055841201 | 0.01127642 | 7.34E−07 |
| imm_12_39012147 | 12 | 40725880.00 | A | G | −0.068214401 | 0.013792304 | 7.58E−07 |
| imm_12_38973921 | 12 | 40687654.00 | A | G | 0.051385582 | 0.010395006 | 7.68E−07 |
| imm_12_38976973 | 12 | 40690706.00 | C | A | 0.051172946 | 0.010391242 | 8.45E−07 |
| imm_12_38830906 | 12 | 40544639.00 | G | C | −0.050100776 | 0.01017824 | 8.55E−07 |
| imm_6_138078865 | 6 | 138037172.00 | A | G | −0.05201299 | 0.010570605 | 8.63E−07 |
| imm_12_38963296 | 12 | 40677029.00 | A | C | 0.051151078 | 0.010401235 | 8.75E−07 |
| imm_5_150418278 | 5 | 150438085.00 | A | G | −0.05726395 | 0.011655237 | 8.96E−07 |
| imm_12_38887209 | 12 | 40600942.00 | G | A | 0.04862081 | 0.009929551 | 9.75E−07 |
| rs7404095 | 16 | 23864590.00 | A | G | −0.048677019 | 0.009942513 | 9.79E−07 |
| rs3088114 | 7 | 6628242.00 | G | A | 0.050886611 | 0.010406434 | 1.01E−06 |
| imm_12_38961875 | 12 | 40675608.00 | A | G | 0.050788723 | 0.010394714 | 1.03E−06 |
| imm_2_234047749 | 2 | 234383010.00 | A | G | 0.062599424 | 0.012882986 | 1.18E−06 |
| imm_12_38699250 | 12 | 40412983.00 | A | C | −0.051089753 | 0.010548275 | 1.28E−06 |
| imm_12_38718529 | 12 | 40432262.00 | A | G | −0.051186784 | 0.010592849 | 1.35E−06 |
| imm_5_150301770 | 5 | 150321577.00 | C | A | 0.048330193 | 0.010006068 | 1.36E−06 |
| imm_12_38879670 | 12 | 40593403.00 | G | A | 0.048009394 | 0.009943121 | 1.38E−06 |
| imm_12_38847946 | NA | NA | NA | NA | −0.051265859 | 0.010620534 | 1.39E−06 |
| imm_2_234043484 | 2 | 234378745.00 | A | G | 0.06214235 | 0.012910065 | 1.48E−06 |
| imm_12_38667030 | 12 | 40380763.00 | G | A | −0.047476541 | 0.009867941 | 1.50E−06 |
| imm_5_150272810 | 5 | 150292617.00 | G | A | −0.050752315 | 0.010562147 | 1.55E−06 |
| rs4755450 | 11 | 36363575.00 | A | G | −0.049697082 | 0.010356825 | 1.60E−06 |
| imm_2_233968747 | 2 | 234304008.00 | A | G | 0.131168946 | 0.027340794 | 1.61E−06 |
| imm_6_138074926 | 6 | 138033233.00 | A | C | −0.047527973 | 0.009909694 | 1.62E−06 |
| imm_6_138076336 | 6 | 138034643.00 | G | A | −0.047042158 | 0.009817568 | 1.65E−06 |
| imm_5_150415838 | 5 | 150435645.00 | G | A | −0.055779709 | 0.011651103 | 1.69E−06 |
| imm_12_39053629 | NA | NA | NA | NA | −0.055722311 | 0.01165229 | 1.73E−06 |
| imm_12_38957265 | 12 | 40670998.00 | A | C | −0.066295573 | 0.013886907 | 1.81E−06 |
| imm_6_138068047 | 6 | 138026354.00 | A | G | −0.050305934 | 0.010551303 | 1.86E−06 |
| imm_6_138060368 | 6 | 138018675.00 | A | G | 0.046865343 | 0.009834341 | 1.88E−06 |
| imm_12_38658287 | 12 | 40372020.00 | C | A | −0.046984629 | 0.009869333 | 1.93E−06 |
| imm_12_38832219 | 12 | 40545952.00 | G | A | −0.093078639 | 0.019565473 | 1.96E−06 |
| imm_12_38820825 | 12 | 40534558.00 | G | A | −0.092107017 | 0.019376177 | 2.00E−06 |
| imm_2_234038440 | NA | NA | NA | NA | 0.146313482 | 0.030784096 | 2.01E−06 |
| imm_12_38838718 | 12 | 40552451.00 | G | C | −0.048415572 | 0.010187208 | 2.01E−06 |
| imm_12_38825125 | 12 | 40538858.00 | G | A | −0.092376772 | 0.019456798 | 2.06E−06 |
| imm_12_38729159 | 12 | 40442892.00 | G | A | −0.0584564 | 0.012314957 | 2.07E−06 |
| imm_6_138181233 | 6 | 138139540.00 | A | G | −0.10120639 | 0.021335478 | 2.10E−06 |
| imm_12_38833646 | 12 | 40547379.00 | A | G | −0.091905438 | 0.019462993 | 2.33E−06 |
| imm_2_234114492 | 2 | 234449753.00 | G | A | 0.061093546 | 0.012981076 | 2.52E−06 |
| imm_12_38838523 | 12 | 40552256.00 | G | A | −0.048553111 | 0.010348894 | 2.71E−06 |
| imm_12_38966138 | 12 | 40679871.00 | A | G | 0.050157117 | 0.010712298 | 2.84E−06 |
| imm_2_233808460 | 2 | 234143721.00 | A | G | 0.15695094 | 0.033633323 | 3.06E−06 |
| imm_12_39010761 | 12 | 40724494.00 | C | A | −0.046033818 | 0.009887494 | 3.23E−06 |
| imm_12_38666068 | 12 | 40379801.00 | A | C | 0.051273079 | 0.011014503 | 3.24E−06 |
| imm_6_138172465 | 6 | 138130772.00 | A | G | −0.098491344 | 0.021160714 | 3.25E−06 |
| imm_2_233993041 | 2 | 234328302.00 | A | G | 0.07344669 | 0.015811066 | 3.40E−06 |
| imm_5_150295884 | 5 | 150315691.00 | A | G | 0.046418491 | 0.010003156 | 3.48E−06 |
| imm_12_39040369 | 12 | 40754102.00 | G | A | 0.224711214 | 0.048449265 | 3.52E−06 |
| imm_6_138020079 | 6 | 137978386.00 | G | A | 0.057751122 | 0.01246844 | 3.63E−06 |
| rs2267364 | 22 | 37677404.00 | A | G | −0.052426552 | 0.011323228 | 3.66E−06 |
| imm_6_138067965 | 6 | 138026272.00 | A | G | −0.048952793 | 0.010573909 | 3.66E−06 |
| imm_5_150279814 | 5 | 150299621.00 | G | A | 0.045661658 | 0.009867066 | 3.70E−06 |
| imm_6_138015797 | 6 | 137974104.00 | G | A | 0.057760851 | 0.01248617 | 3.73E−06 |
| imm_12_38704491 | 12 | 40418224.00 | A | G | −0.047870652 | 0.010357381 | 3.80E−06 |
| imm_6_138156959 | 6 | 138115266.00 | G | A | −0.046073024 | 0.009980031 | 3.90E−06 |
| rs12295535 | 11 | 36432024.00 | A | G | 0.140346518 | 0.030412372 | 3.94E−06 |
| imm_5_150291871 | 5 | 150311678.00 | G | A | 0.04552974 | 0.009866959 | 3.94E−06 |
| imm_5_150276201 | 5 | 150296008.00 | A | G | 0.045529324 | 0.00987069 | 3.98E−06 |
| imm_5_150278308 | 5 | 150298115.00 | A | G | 0.045501513 | 0.00986692 | 4.00E−06 |
| imm_12_38703075 | 12 | 40416808.00 | G | A | −0.047849882 | 0.010392748 | 4.14E−06 |
| imm_5_150277541 | 5 | 150297348.00 | A | G | 0.045427096 | 0.009870345 | 4.18E−06 |
| imm_5_150292277 | 5 | 150312084.00 | A | G | 0.045289639 | 0.009840722 | 4.18E−06 |
| imm_2_234022252 | 2 | 234357513.00 | G | A | 0.070304557 | 0.015276273 | 4.18E−06 |
| imm_6_138315317 | 6 | 138273624.00 | A | C | −0.070164568 | 0.015260416 | 4.27E−06 |
| imm_5_150296454 | 5 | 150316261.00 | G | A | 0.045975337 | 0.010002821 | 4.30E−06 |
| imm_5_150414614 | 5 | 150434421.00 | A | G | −0.053622703 | 0.011685643 | 4.46E−06 |
| imm_2_233999251 | 2 | 234334512.00 | G | A | 0.071694323 | 0.015650705 | 4.63E−06 |
| imm_5_150291966 | 5 | 150311773.00 | G | A | 0.045174995 | 0.009866959 | 4.69E−06 |
| imm_5_150291815 | 5 | 150311622.00 | G | A | 0.045136965 | 0.009866959 | 4.77E−06 |
| imm_12_38895848 | 12 | 40609581.00 | A | G | −0.10370029 | 0.022670231 | 4.78E−06 |
| imm_5_150289887 | 5 | 150309694.00 | A | G | 0.04505972 | 0.009866961 | 4.95E−06 |
| imm_12_38839825 | 12 | 40553558.00 | G | A | −0.047733106 | 0.01045765 | 5.01E−06 |
| imm_5_150291561 | 5 | 150311368.00 | C | A | 0.045024238 | 0.009870384 | 5.08E−06 |
| imm_6_138202706 | 6 | 138161013.00 | G | A | 0.047713042 | 0.010469595 | 5.18E−06 |
| imm_5_150271596 | 5 | 150291403.00 | A | G | 0.045040468 | 0.009890446 | 5.27E−06 |
| imm_12_38666302 | 12 | 40380035.00 | A | G | −0.045265036 | 0.009940133 | 5.27E−06 |
| imm_2_234056440 | 2 | 234391701.00 | A | T | −0.163465633 | 0.035935145 | 5.39E−06 |
| imm_5_150291045 | 5 | 150310852.00 | A | G | 0.044851427 | 0.009867129 | 5.48E−06 |
| imm_5_150274013 | 5 | 150293820.00 | G | A | 0.044948261 | 0.009890191 | 5.50E−06 |
| imm_12_38902681 | 12 | 40616414.00 | G | A | −0.108211541 | 0.023853369 | 5.72E−06 |
| imm_5_150279403 | 5 | 150299210.00 | A | G | 0.044666225 | 0.009866826 | 5.99E−06 |
| imm_12_38738503 | 12 | 40452236.00 | G | A | −0.053693683 | 0.011868866 | 6.07E−06 |
| imm_12_38843127 | 12 | 40556860.00 | G | A | −0.048287308 | 0.010682742 | 6.18E−06 |
| imm_12_38851565 | 12 | 40565298.00 | A | C | −0.046441358 | 0.010295863 | 6.46E−06 |
| imm_5_150293216 | 5 | 150313023.00 | C | A | 0.044715979 | 0.009916619 | 6.51E−06 |
| imm_5_150294942 | 5 | 150314749.00 | A | G | 0.044710933 | 0.009936437 | 6.81E−06 |
| imm_5_150294664 | 5 | 150314471.00 | A | G | 0.044542125 | 0.009936107 | 7.37E−06 |
| imm_6_138043754 | 6 | 138002061.00 | A | G | 0.055970043 | 0.012487557 | 7.39E−06 |
| imm_5_150294449 | 5 | 150314256.00 | T | A | 0.044193386 | 0.009913324 | 8.27E−06 |
| ccc0501502950430A0G | 5 | 150314850.00 | G | A | 0.044289646 | 0.009936107 | 8.29E−06 |
| imm_5_150272518 | 5 | 150292325.00 | C | A | 0.044099362 | 0.009893722 | 8.30E−06 |
| imm_5_150273600 | 5 | 150293407.00 | G | A | 0.044081877 | 0.009906566 | 8.60E−06 |
| imm_12_38668966 | 12 | 40382699.00 | G | A | −0.0455455 | 0.010236618 | 8.62E−06 |
| imm_5_150293745 | 5 | 150313552.00 | G | A | 0.044042496 | 0.009913326 | 8.88E−06 |
| imm_6_138023277 | 6 | 137981584.00 | A | G | 0.055655744 | 0.012555768 | 9.31E−06 |

TABLE 2
SNPs Associated with Inflammatory Bowel Disease

| SEQ | | | | | | | | | |
|---|---|---|---|---|---|---|---|---|---|
| ID | | | | | | Minor | Major | | |
| NO | rsID | Chip ID | chr | Locus | Gene | Allele | Allele | Beta | P Value |
| 1 | rs11564258 | imm_12_39078567 | chr12 | LRRK2- | LRRK2\| | A | G | 0.144068462 | 3.2981E−05 |
| | | | | MUC19 | MUC19 | | | | |
| 2 | rs2357623 | imm_16_49251512 | chr16 | NOD2 | NKD1 \| SLIC1 | A | G | −0.005539402 | 0.648994203 |
| 3 | rs2066845 | imm_16_49314041 | chr16 | NOD2 | NOD2 | G | C | −0.03867881 | 0.369865461 |
| 4 | rs5743289 | imm_16_49314275 | chr16 | NOD2 | NOD2 | A | G | 0.001523419 | 0.936602704 |
| 5 | rs72796367 | imm_16_49320272 | chr16 | NOD2 | NOD2 | G | A | −0.107581114 | 0.005831005 |
| 6 | rs6752107 | imm_2_233826187 | chr2 | ATG16L1 | ATG16L1 | G | A | −0.02579317 | 0.014036466 |
| 7 | rs12994997 | imm_2_233838242 | chr2 | ATG16L1 | ATG16L1 | G | A | −0.026639104 | 0.011185389 |
| 8 | rs11741861 | imm_5_150258102 | chr5 | IRGM | ZNF300 | G | A | 0.131116086 | 2.7726E−12 |
| 9 | rs9494844 | imm_6_138025162 | chr6 | | OLIG3 \| | A | C | −0.073397103 | 2.28771E-09 |
| | | | | | LOC100130476 | | | | |
| 10 | rs6918329 | imm_6_138202706 | chr6 | TNFAIP3 | OLIG3 \| | G | A | −0.030804821 | 0.006503716 |
| | | | | | LOC100130476 | | | | |
| 11 | rs7404095 | rs7404095 | chr16 | | PRKCB1 | A | G | −0.075245799 | 1.46487E−12 |

TABLE 3
SNPs Associated with Crohn's Disease

| SEQ ID | | | | | | Minor | Major | | |
|---|---|---|---|---|---|---|---|---|---|
| NO | rsID | Chip ID | chr | Locus | Gene | Allele | Allele | Beta | P Value |
| 1 | rs11564258 | imm_12_39078567 | chr12 | LRRK2-MUC19 | LRRK2 \| MUC19 | A | G | 0.389141609 | 2.17975E−39 |
| 2 | rs2357623 | imm_16_49251512 | chr16 | NOD2 | NKD1 \| SLIC1 | A | G | 0.206419294 | 9.98329E−78 |
| 3 | rs2066845 | imm_16_49314041 | chr16 | NOD2 | NOD2 | G | C | 0.211421604 | 1.6983E−11 |
| 4 | rs5743289 | imm_16_49314275 | chr16 | NOD2 | NOD2 | A | G | 0.396201009 | 8.1253E−126 |
| 5 | rs72796367 | imm_16_49320272 | chr16 | NOD2 | NOD2 | G | A | 0.226038595 | 4.71001E−12 |
| 6 | rs6752107 | imm_2_233826187 | chr2 | ATG16L1 | ATG16L1 | G | A | −0.229578892 | 6.9368E−118 |
| 7 | rs12994997 | imm_2_233838242 | chr2 | ATG16L1 | ATG16L1 | G | A | −0.229550418 | 5.9458E−118 |
| 8 | rs11741861 | imm_5_150258102 | chr5 | IRGM | ZNF300 | G | A | 0.251723092 | 1.35845E−49 |
| 9 | rs9494844 | imm_6_138025162 | chr6 | | OLIG3 \| LOC100130476 | A | C | −0.076161445 | 3.31349E−11 |
| 10 | rs6918329 | imm_6_138202706 | chr6 | TNFAIP3 | OLIG3 \| LOC100130476 | G | A | 0.047713042 | 5.18164E−06 |

TABLE 4
SNPs Associated with Ulcerative Colitis

| SEQ ID | | | | | | Minor | Major | | |
|---|---|---|---|---|---|---|---|---|---|
| NO | rsID | Chip ID | chr | Locus | Gene | Allele | Allele | Beta | P Value |
| 1 | rs11564258 | imm_12_39078567 | chr12 | LRRK2-MUC19 | LRRK2 \| MUC19 | A | G | 0.284395247 | 2.28694E−28 |
| 2 | rs2357623 | imm_16_49251512 | chr16 | NOD2 | NKD1 \| SLIC1 | A | G | 0.108467352 | 4.94236E−32 |
| 3 | rs2066845 | imm_16_49314041 | chr16 | NOD2 | NOD2 | G | C | 0.114126171 | 7.22153E−05 |
| 4 | rs5743289 | imm_16_49314275 | chr16 | NOD2 | NOD2 | A | G | 0.221200293 | 9.88394E−58 |
| 5 | rs72796367 | imm_16_49320272 | chr16 | NOD2 | NOD2 | G | A | 0.082691748 | 0.003391837 |
| 6 | rs6752107 | imm_2_233826187 | chr2 | ATG16L1 | ATG16L1 | G | A | −0.131893309 | 9.99554E−60 |
| 7 | rs12994997 | imm_2_233838242 | chr2 | ATG16L1 | ATG16L1 | G | A | −0.132711316 | 1.8915E−60 |
| 8 | rs11741861 | imm_5_150258102 | chr5 | IRGM | ZNF300 | G | A | 0.188033357 | 1.27584E−39 |
| 9 | rs9494844 | imm_6_138025162 | chr6 | | OLIG3 \| LOC100130476 | A | C | −0.072891179 | 9.39947E−15 |
| 10 | rs6918329 | imm_6_138202706 | chr6 | TNFAIP3 | OLIG3 \| LOC100130476 | G | A | 0.010588879 | 0.220475463 |
| 11 | rs7404095 | rs7404095 | chr16 | | PRKCB1 | A | G | −0.059830094 | 2.62181E−13 |

### Example 2. SNPs Associated with Subclinical Phenotypes in Patients with CD

Multiple large-scale case-control association studies involving Inflammatory Bowel Crohn's disease (CD) and various subclinical phenotypes of CD in Caucasian populations using gene-based single nucleotide polymorphism (SNP) markers were performed. The studies included patients recruited at the Cedars-Sinai Inflammatory Bowel Disease Centers with Age at diagnosis (n=2566), ANCA levels/presence (n=2315), Cbirl levels/presence (n=2356), 12 levels/presence (n=1805), ASCA levels/presence (n=2290), Ompc levels/presence (n=2321), Stricturing and penetrating (n=2316), stricturing (n=1738), penetrating (n=1691), ileal disease (n=2307), Time to First Surgery (n=2434), and perianal CD (n=2187). The “Age of diagnosis” as used herein refers to between 1-5, 5-10, 10-15, 15-20, 20-25, 25-30, 30-35, 35-40, 40-45, 45-50, 50-55, 55-60, 60-65, 65-70, 70-75, 75-80, 80-85, or 85-90 years of age. .Table 5 provides SNPs associated with the above subclinical phenotypes associated with CD.

TABLE 5
SNPs Associated with Subclinical Phenotypes in Patients with CD

| Subclinical Phenotype | P value | OR |
|---|---|---|
| imm_12_39078567 (SEQ ID NO: 1) | | |
| Age at Diagnosis | 0.122256071 | NA |
| ANCA (High level) | 0.31252474 | NA |
| ANCA (Presence v. Absence) | 0.778856904 | 1.041088668 |
| Stricturing & Penetrating v. Non-Stricturing & Non-Penetrating | 0.138548355 | 1.204318834 |
| Stricturing v. Non-Stricturing | 0.12838819 | 1.251965684 |
| Penetrating v. Non-Penetrating | 0.341624972 | 1.15744607 |
| Cbir1 (High level) | 0.728800495 | NA |
| Cbir1 (Presence v. Absence) | 0.943223147 | 1.009148135 |
| I2 (High level) | 0.312744648 | NA |
| I2 (Presence v. Absence) | 0.789599275 | 1.039803659 |
| IgA ASCA (High level) | 0.866377885 | NA |
| IgA ASCA (Presence v. Absence) | 0.49447574 | 0.910324989 |
| IgG ASCA (High level) | 0.347322072 | NA |
| IgG ASCA (Presence v. Absence) | 0.74661289 | 1.043635316 |
| Ilealcolonic/Ileal v. Colonic Disease | 0.76853167 | 1.048185926 |
| Ileal Disease v. Colonic Disease | 0.046696511 | 1.443702066 |
| Ileal Disease v. Colonic Disease | 0.438853915 | 0.877786052 |
| Ompc (High level) | 0.295752526 | NA |
| Ompc (Presence v. Absence) | 0.895118238 | 0.981026129 |
| Perianal CD | 0.256237748 | 0.849599385 |
| Shorter Time from First Diagnosis to First Surgery | 0.875220611 | 1.019666299 |
| imm_16_49251512 (SEQ ID NO: 6) | | |
| Age at Diagnosis | 0.104552109 | NA |
| ANCA (High level) | 0.143344149 | NA |
| ANCA (Presence v. Absence) | 0.336733181 | 0.931274715 |
| Stricturing & Penetrating v. Non-Stricturing & Non-Penetrating | 0.115915372 | 1.107779186 |
| Stricturing v. Non-Stricturing | 0.303455515 | 1.08508732 |
| Penetrating v. Non-Penetrating | 0.121080714 | 1.130542275 |
| Cbir1 (High level) | 0.014291458 | NA |
| Cbir1 (Presence v. Absence) | 0.032325905 | 1.147845581 |
| I2 (High level) | 0.877411648 | NA |
| I2 (Presence v. Absence) | 0.320562388 | 0.928456362 |
| IgA ASCA (High level) | 0.06300576 | NA |
| IgA ASCA (Presence v. Absence) | 0.054248296 | 1.14132685 |
| IgG ASCA (High level) | 0.039635415 | NA |
| IgG ASCA (Presence v. Absence) | 0.055946744 | 1.138008136 |
| Ilealcolonic/Ileal v. Colonic Disease | 0.337410217 | 1.082220963 |
| Ileal Disease v. Colonic Disease | 0.03102855 | 1.238312142 |
| Ileal Disease v. Colonic Disease | 0.798698348 | 1.022141176 |
| Ompc (High level) | 0.38973421 | NA |
| Ompc (Presence v. Absence) | 0.084562162 | 1.134000054 |
| Perianal CD | 0.554100471 | 0.957944454 |
| Shorter Time from First Diagnosis to First Surgery | 0.03526004 | 1.143084612 |
| imm_16_49314041 (SEQ ID NO: 3) | | |
| Age at Diagnosis | 0.030888248 | NA |
| ANCA (High level) | 0.098361711 | NA |
| ANCA (Presence v. Absence) | 0.060965273 | 0.742123637 |
| Stricturing & Penetrating v. Non-Stricturing & Non-Penetrating | 0.272499936 | 1.151539837 |
| Stricturing v. Non-Stricturing | 0.789203073 | 0.957759235 |
| Penetrating v. Non-Penetrating | 0.034627665 | 1.371551298 |
| Cbir1 (High level) | 0.023810244 | NA |
| Cbir1 (Presence v. Absence) | 0.227074659 | 1.16912378 |
| I2 (High level) | 0.197414244 | NA |
| I2 (Presence v. Absence) | 0.81624873 | 0.966259515 |
| IgA ASCA (High level) | 0.002232358 | NA |
| IgA ASCA (Presence v. Absence) | 0.017500825 | 1.372430743 |
| IgG ASCA (High level) | 2.54E−07 | NA |
| IgG ASCA (Presence v. Absence) | 4.02E−06 | 1.836315926 |
| Ilealcolonic/Ileal v. Colonic Disease | 0.011172184 | 1.618556215 |
| Ileal Disease v. Colonic Disease | 0.000198287 | 2.21214919 |
| Ileal Disease v. Colonic Disease | 0.099485701 | 1.380551455 |
| Ompc (High level) | 0.43396744 | NA |
| Ompc (Presence v. Absence) | 0.320286478 | 1.150451809 |
| Perianal CD | 0.322861459 | 1.148071707 |
| Shorter Time from First Diagnosis to First Surgery | 0.275196907 | 1.14651152 |
| imm_16_49314275 (SEQ ID NO: 4) | | |
| Age at Diagnosis | 0.004706789 | NA |
| ANCA (High level) | 0.903154792 | NA |
| ANCA (Presence v. Absence) | 0.849722017 | 0.985437223 |
| Stricturing & Penetrating v. Non-Stricturing & Non-Penetrating | 0.00016302 | 1.292842996 |
| Stricturing v. Non-Stricturing | 0.003031771 | 1.273026537 |
| Penetrating v. Non-Penetrating | 0.000844755 | 1.319619709 |
| Cbir1 (High level) | 0.068609344 | NA |
| Cbir1 (Presence v. Absence) | 0.024653162 | 1.163410809 |
| I2 (High level) | 0.502599553 | NA |
| I2 (Presence v. Absence) | 0.058152465 | 1.159352262 |
| IgA ASCA (High level) | 7.80E−08 | NA |
| IgA ASCA (Presence v. Absence) | 1.11E−07 | 1.461724698 |
| IgG ASCA (High level) | 2.14E−07 | NA |
| IgG ASCA (Presence v. Absence) | 1.01E−06 | 1.412939383 |
| Ilealcolonic/Ileal v. Colonic Disease | 0.000264182 | 1.390521204 |
| Ileal Disease v. Colonic Disease | 3.32E−06 | 1.644810393 |
| Ileal Disease v. Colonic Disease | 0.006720882 | 1.291729465 |
| Ompc (High level) | 0.022771083 | NA |
| Ompc (Presence v. Absence) | 0.059140225 | 1.155358806 |
| Perianal CD | 0.491573202 | 0.949720982 |
| Shorter Time from First Diagnosis to First Surgery | 0.007004352 | 1.195813516 |
| imm_16_49320272 (SEQ ID NO: 5) | | |
| Age at Diagnosis | 0.540173487 | NA |
| ANCA (High level) | 0.49082237 | NA |
| ANCA (Presence v. Absence) | 0.617666642 | 1.100154859 |
| Stricturing & Penetrating v. Non-Stricturing & Non-Penetrating | 0.857751847 | 1.031769742 |
| Stricturing v. Non-Stricturing | 0.477806289 | 1.154219921 |
| Penetrating v. Non-Penetrating | 0.648205587 | 0.900619565 |
| Cbir1 (High level) | 0.220318463 | NA |
| Cbir1 (Presence v. Absence) | 0.055777639 | 0.718782414 |
| I2 (High level) | 0.230735493 | NA |
| I2 (Presence v. Absence) | 0.319918307 | 1.214644438 |
| IgA ASCA (High level) | 0.575231886 | NA |
| IgA ASCA (Presence v. Absence) | 0.31837683 | 0.82857951 |
| IgG ASCA (High level) | 0.322045841 | NA |
| IgG ASCA (Presence v. Absence) | 0.434377064 | 0.866818352 |
| Ilealcolonic/Ileal v. Colonic Disease | 0.198187568 | 1.367316011 |
| Ileal Disease v. Colonic Disease | 0.307417502 | 1.326242822 |
| Ileal Disease v. Colonic Disease | 0.22130683 | 1.36301993 |
| Ompc (High level) | 0.481240402 | NA |
| Ompc (Presence v. Absence) | 0.197026716 | 1.272860825 |
| Perianal CD | 0.933505392 | 0.984240053 |
| Shorter Time from First Diagnosis to First Surgery | 0.637017351 | 0.922600761 |
| imm_2_233826187 (SEQ ID NO: 6) | | |
| Age at Diagnosis | 0.019134942 | NA |
| ANCA (High level) | 0.203445501 | NA |
| ANCA (Presence v. Absence) | 0.791444917 | 1.018464485 |
| Stricturing & Penetrating v. Non-Stricturing & Non-Penetrating | 0.006097284 | 0.846906937 |
| Stricturing v. Non-Stricturing | 0.069298368 | 0.875030768 |
| Penetrating v. Non-Penetrating | 0.006316386 | 0.812436791 |
| Cbir1 (High level) | 0.769181666 | NA |
| Cbir1 (Presence v. Absence) | 0.435230923 | 0.954062779 |
| I2 (High level) | 0.227503946 | NA |
| I2 (Presence v. Absence) | 0.512571086 | 0.9550564 |
| IgA ASCA (High level) | 0.079825984 | NA |
| IgA ASCA (Presence v. Absence) | 0.052576237 | 0.880428485 |
| IgG ASCA (High level) | 0.002825304 | NA |
| IgG ASCA (Presence v. Absence) | 0.00258127 | 0.822578786 |
| Ilealcolonic/Ileal v. Colonic Disease | 8.29E−05 | 0.743382681 |
| Ileal Disease v. Colonic Disease | 0.002119838 | 0.748678577 |
| Ileal Disease v. Colonic Disease | 0.000107307 | 0.736621182 |
| Ompc (High level) | 0.41918304 | NA |
| Ompc (Presence v. Absence) | 0.867924955 | 1.01159327 |
| Perianal CD | 0.886766562 | 1.009617819 |
| Shorter Time from First Diagnosis to First Surgery | 0.036540834 | 0.883755342 |
| imm_2_233838242 (SEQ ID NO: 7) | | |
| Age at Diagnosis | 0.022852465 | NA |
| ANCA (High level) | 0.17494154 | NA |
| ANCA (Presence v. Absence) | 0.776887056 | 1.019757848 |
| Stricturing & Penetrating v. Non-Stricturing & Non-Penetrating | 0.006804209 | 0.84906255 |
| Stricturing v. Non-Stricturing | 0.081566647 | 0.880168445 |
| Penetrating v. Non-Penetrating | 0.005958725 | 0.811555429 |
| Cbir1 (High level) | 0.854997129 | NA |
| Cbir1 (Presence v. Absence) | 0.434393835 | 0.954056492 |
| I2 (High level) | 0.236589872 | NA |
| I2 (Presence v. Absence) | 0.48595978 | 0.952313795 |
| IgA ASCA (High level) | 0.086420157 | NA |
| IgA ASCA (Presence v. Absence) | 0.064612421 | 0.885933031 |
| IgG ASCA (High level) | 0.003135093 | NA |
| IgG ASCA (Presence v. Absence) | 0.00285243 | 0.8245458 |
| Ilealcolonic/Ileal v. Colonic Disease | 0.000150175 | 0.752063769 |
| Ileal Disease v. Colonic Disease | 0.002606654 | 0.75364079 |
| Ileal Disease v. Colonic Disease | 0.000210369 | 0.747053156 |
| Ompc (High level) | 0.414851495 | NA |
| Ompc (Presence v. Absence) | 0.908141514 | 1.00801554 |
| Perianal CD | 0.868250217 | 1.011188504 |
| Shorter Time from First Diagnosis to First Surgery | 0.034225129 | 0.882578253 |
| imm_5_150258102 (SEQ ID NO: 8) | | |
| Age at Diagnosis | 0.843744329 | NA |
| ANCA (High level) | 0.748606381 | NA |
| ANCA (Presence v. Absence) | 0.385770551 | 0.915370776 |
| Stricturing & Penetrating v. Non-Stricturing & Non-Penetrating | 0.090103093 | 1.161123742 |
| Stricturing v. Non-Stricturing | 0.246105587 | 1.129935911 |
| Penetrating v. Non-Penetrating | 0.102162251 | 1.190301949 |
| Cbir1 (High level) | 0.986555148 | NA |
| Cbir1 (Presence v. Absence) | 0.274783393 | 1.101183016 |
| I2 (High level) | 0.495340867 | NA |
| I2 (Presence v. Absence) | 0.86962572 | 1.016710916 |
| IgA ASCA (High level) | 0.691167889 | NA |
| IgA ASCA (Presence v. Absence) | 0.362212537 | 0.917166261 |
| IgG ASCA (High level) | 0.516535284 | NA |
| IgG ASCA (Presence v. Absence) | 0.508642146 | 0.940592963 |
| Ilealcolonic/Ileal v. Colonic Disease | 0.602822402 | 1.060275335 |
| Ileal Disease v. Colonic Disease | 0.691005152 | 1.054653664 |
| Ileal Disease v. Colonic Disease | 0.624246837 | 1.059029001 |
| Ompc (High level) | 0.395508911 | NA |
| Ompc (Presence v. Absence) | 0.235839652 | 1.124082553 |
| Perianal CD | 0.874178504 | 0.984639691 |
| Shorter Time from First Diagnosis to First Surgery | 0.060375477 | 1.174193485 |
| imm_6_138025162 (SEQ ID NO: 9) | | |
| Age at Diagnosis | 0.773014339 | NA |
| ANCA (High level) | 0.96637959 | NA |
| ANCA (Presence v. Absence) | 0.909134187 | 1.009213119 |
| Stricturing & Penetrating v. Non-Stricturing & Non-Penetrating | 0.709435082 | 0.974060228 |
| Stricturing v. Non-Stricturing | 0.946936915 | 0.994412118 |
| Penetrating v. Non-Penetrating | 0.576050476 | 0.952176027 |
| Cbir1 (High level) | 0.76237939 | NA |
| Cbir1 (Presence v. Absence) | 0.831526862 | 0.985160326 |
| I2 (High level) | 0.510635212 | NA |
| I2 (Presence v. Absence) | 0.487447837 | 1.057860125 |
| IgA ASCA (High level) | 0.906775261 | NA |
| IgA ASCA (Presence v. Absence) | 0.508651149 | 0.951002862 |
| IgG ASCA (High level) | 0.809848871 | NA |
| IgG ASCA (Presence v. Absence) | 0.564720863 | 0.957978412 |
| Ilealcolonic/Ileal v. Colonic Disease | 0.263590149 | 1.105464684 |
| Ileal Disease v. Colonic Disease | 0.04287932 | 1.244027553 |
| Ileal Disease v. Colonic Disease | 0.592640292 | 1.051258357 |
| Ompc (High level) | 0.802692993 | NA |
| Ompc (Presence v. Absence) | 0.909794798 | 0.990911401 |
| Perianal CD | 0.399433324 | 0.935613785 |
| Shorter Time from First Diagnosis to First Surgery | 0.282302594 | 0.929025707 |
| imm_6_138202706 (SEQ ID NO: 10) | | |
| Age at Diagnosis | 0.542977462 | NA |
| ANCA (High level) | 0.550079678 | NA |
| ANCA (Presence v. Absence) | 0.676660505 | 1.0296575 |
| Stricturing & Penetrating v. Non-Stricturing & Non-Penetrating | 0.8439828 | 0.988022286 |
| Stricturing v. Non-Stricturing | 0.960961827 | 0.996394819 |
| Penetrating v. Non-Penetrating | 0.762822209 | 0.977334388 |
| Cbir1 (High level) | 0.532994437 | NA |
| Cbir1 (Presence v. Absence) | 0.525375198 | 1.039488258 |
| I2 (High level) | 0.204693972 | NA |
| I2 (Presence v. Absence) | 0.756643643 | 0.978314018 |
| IgA ASCA (High level) | 0.630036623 | NA |
| IgA ASCA (Presence v. Absence) | 0.988816983 | 0.999074187 |
| IgG ASCA (High level) | 0.546243185 | NA |
| IgG ASCA (Presence v. Absence) | 0.326767348 | 1.065607543 |
| Ilealcolonic/Ileal v. Colonic Disease | 0.908504429 | 1.008875751 |
| Ileal Disease v. Colonic Disease | 0.686910915 | 1.039104155 |
| Ileal Disease v. Colonic Disease | 0.953097604 | 0.995295322 |
| Ompc (High level) | 0.318658833 | NA |
| Ompc (Presence v. Absence) | 0.699951623 | 1.027352674 |
| Perianal CD | 0.416731262 | 0.945844201 |
| Shorter Time from First Diagnosis to First Surgery | 0.326898455 | 0.943027447 |
| rs7404095 (SEQ ID NO: 11) | | |
| Age at Diagnosis | 0.357185338 | NA |
| ANCA (High level) | 0.569568072 | NA |
| anca.pos | 0.905445905 | 1.008120442 |
| Stricturing & Penetrating v. Non-Stricturing & Non-Penetrating | 0.382204083 | 1.053954236 |
| Stricturing v. Non-Stricturing | 0.856435686 | 0.986909537 |
| Penetrating v. Non-Penetrating | 0.096509538 | 1.131876475 |
| Cbir1 (High level) | 0.337778943 | NA |
| Cbir1 (Presence v. Absence) | 0.207599582 | 0.928132545 |
| I2 (High level) | 0.435650519 | NA |
| I2 (Presence v. Absence) | 0.380240435 | 0.941195259 |
| IgA ASCA (High level) | 0.724665876 | NA |
| IgA ASCA (Presence v. Absence) | 0.586054573 | 0.965709084 |
| IgG ASCA (High level) | 0.543893652 | NA |
| IgG ASCA (Presence v. Absence) | 0.429552494 | 1.050934054 |
| Ileal Disease v. Colonic Disease | 0.872863472 | 1.012131958 |
| Ileal Disease v. Colonic Disease | 0.726586924 | 0.968786128 |
| Ileal Disease v. Colonic Disease | 0.683719538 | 1.032916228 |
| Ompc (High level) | 0.703487767 | NA |
| Ompc (Presence v. Absence) | 0.503618876 | 1.046517576 |
| Perianal CD | 0.337563463 | 0.937753069 |
| Shorter Time from First Diagnosis to First Surgery | 0.299326191 | 0.941052493 |

### Example 3. Calculating a Polygenic Risk Score

Genetic patient selection criteria for treatment with a RIP2K inhibitor is determined by calculating a polygenic risk score (PRS) for all patients within the Precision IBD database based on a defined set of polymorphisms (SNPs) representing the risk genotype. The PRS is based on SNPs within the multiple genes involved in the RIPK2 pathway and associated with a risk of having, or developing inflammatory bowel diseases, including Crohn's disease and ulcerative colitis, and their associated weights (e.g., beta value). In this example two exemplary methods of calculating a PRS are provided. In each example, numerical values are assigned to genotypes observed in a subject and reference population. A homozygous genotype for a risk allele within a SNV (RR) is assigned a numerical value 2; a heterozygous genotype for a risk allele within a SN (R) is assigned a numerical value 1; a genotype that is nonrisk (N) is assigned a numerical value 0.

Method 1

The PRS is calculated as a normalized weighted sum of the number of risk alleles carried by each individual (0, 1, or 2) at each risk loci, with weights proportional to the odds ratio or beta value of disease association. The PRS is binomially distributed based on distribution parameters (e.g. mean and variance) within the population and gives an individual risk assessment for variants within this signature. Table 6 provides exemplary calculations using method 1 described herein. Each row represents a different subject. Each column represents the total number of risk alleles per SNP detected in a sample obtained from each subject. For purposes of this example SNP 1 is imm_12_39078567, SNP 2 is imm_16_49251512, SNP 3 is imm_16_49314041, SNP 4 is imm_16_49314275, SNP 5 is imm_16-49320272, SNP 6 is imm_2_233826187, SNP 7 is imm_2_233838242, SNP 8 is imm_5_150258102, SNP 9 is imm_6_138025162, SNP 10 is imm_6_138202706, and SNP 11 is rs7404095. The following table is illustrative only. For example, a PRS may be calculated using 1 SNP, 2 SNPs, 3 SNPs, 4 SNPs, 5 SNPs, or more. Any combination of SNPs, disclosed herein, or SNP in linkage disequilibrium therewith may be used.

TABLE 6

| SNP | SNP | SNP | SNP | SNP | SNP | SNP | SNP | SNP | SNP | SNP | |
|---|---|---|---|---|---|---|---|---|---|---|---|
| 1 | 2 | 3 | 4 | 5 | 6 | 7 | 8 | 9 | 10 | 11 | score_all_CD |
| 0 | 0 | 0 | 0 | 1 | 2 | 2 | 0 | 0 | 1 | 0 | −1.200190726 |
| 0 | 0 | 0 | 0 | 0 | 0 | 0 | 0 | 0 | 1 | 1 | 0.087892707 |
| 0 | 1 | 0 | 1 | 0 | 0 | 0 | 0 | 2 | 1 | 1 | 0.989185443 |
| 0 | 0 | 0 | 0 | 0 | 2 | 2 | 0 | 1 | 1 | 2 | −1.999918142 |
| 0 | 1 | 0 | 0 | 0 | 0 | 0 | 1 | 1 | 1 | 0 | 0.949876097 |
| 0 | 0 | 0 | 0 | 0 | 1 | 1 | 2 | 0 | 1 | 0 | 0.274024621 |
| 0 | 0 | 0 | 0 | 0 | 0 | 0 | 1 | 1 | 0 | 2 | 0.246358587 |
| 1 | 1 | 1 | 0 | 0 | 1 | 1 | 0 | 0 | 2 | 2 | 1.561095127 |
| 0 | 1 | 0 | 1 | 0 | 1 | 1 | 0 | 0 | 1 | 0 | 0.472526567 |
| 0 | 0 | 1 | 1 | 0 | 0 | 0 | 0 | 1 | 2 | 0 | 1.344568262 |

Method 2

The PRS is calculated as the weighted sum of the number of risk alleles carried by each individual (0, 1, or 2) at each risk loci, divided by a total number of genetic variants used in the model. The same calculations are performed for each individual belonging to a reference group, thereby generating a range of raw scores (observed range). A percentage of risk relative to the reference population is calculated.

### Example 4. Treating an Crohn's Disease or Ulcerative Colitis

Moderate to severe Crohn's disease and/or ulcerative colitis is treated in a subject with an antagonist of RIPK2 activity or expression (e.g., an inhibitor of RIPK2), by first, determining the genotype of the subject. Optionally, the subject is, or is susceptible to be, non-responsive to certain therapies such as anti-TNF, steroids, or immunomodulators, such as those disclosed herein. A sample of whole blood is obtained from the subject. An assay is performed on the sample obtained from the subject to detect a presence or absence of a genotype comprising rs11564258A, rs2357623A, rs2066845G, rs5743289A, rs72796367G, rs6752107A, rs12994997A, rs11741861G, rs9494844C, rs6918329G, and/or rs7404095G by Illumina ImmunoArray or polymerase chain reaction (PCR) under standard hybridization conditions. Alternatively, an assay is performed on the sample obtained from the subject to detect a presence or absence of a genotype comprising SEQ ID NO: 1 (N=A), SEQ ID NO: 2 (N=A), SEQ ID NO: 3 (N=G), SEQ ID NO: 4 (N=A), SEQ ID NO: 5 (N=G), SEQ ID NO: 6 (N=A), SEQ ID NO: 7 (N=A), SEQ ID NO: 8 (N=G), SEQ ID NO: 9 (N=C), SEQ ID NO: 10 (N=G), and/or SEQ ID NO: 11 (N=G). In some instances, the is performed to detect a genotype comprising rs5743289A (SEQ ID NO: 4), rs6752107A (SEQ ID NO: 6), and rs12994997A (SEQ ID NO: 7).

The subject is determined to have, or be at risk for developing, Crohn's disease and/or ulcerative colitis if the genotype is detected in the sample obtained from the subject. A therapeutically effective amount of an antagonist of RIPK2 activity or expression is administered to the subject, provided the subject is determined to have the genotype.

### Example 5. Treating Inflammatory Bowel Disease

Inflammatory bowel disease is treated in a subject with an antagonist of RIPK2 activity or expression (e.g., an inhibitor of RIPK2), by first, determining the genotype of the subject. Optionally, the subject is, or is susceptible to be, non-responsive to certain therapies such as anti-TNF, steroids, or immunomodulators, such as those disclosed herein. A sample of whole blood is obtained from the subject. An assay is performed on the sample obtained from the subject to detect a presence or absence of a genotype comprising rs11564258A, rs2357623G, rs2066845C, rs5743289A, rs72796367A, rs6752107A, rs12994997A, rs11741861G, rs9494844C, rs6918329A, and/or rs7404095G by Illumina ImmunoArray or polymerase chain reaction (PCR) under standard hybridization conditions. Alternatively, an assay is performed on the sample obtained from the subject to detect a presence or absence of a genotype comprising SEQ ID NO: 1 (N=A), SEQ ID NO: 2 (N=G), SEQ ID NO: 3 (N=C), SEQ ID NO: 4 (N=A), SEQ ID NO: 5 (N=A), SEQ ID NO: 6 (N=A), SEQ ID NO: 7 (N=A), SEQ ID NO: 8 (N=G), SEQ ID NO: 9 (N=C), SEQ ID NO: 10 (N=A), and/or SEQ ID NO: 11 (N=G). In some instances, the is performed to detect a genotype comprising rs5743289A (SEQ ID NO: 4), rs6752107A (SEQ ID NO: 6), and rs12994997A (SEQ ID NO: 7).

The subject is determined to have, or be at risk for developing, inflammatory bowel disease if the genotype is detected in the sample obtained from the subject. A therapeutically effective amount of an antagonist of RIPK2 activity or expression is administered to the subject, provided the subject is determined to have the genotype.

### Example 6. Phase 1A Clinical Trial

A phase 1A clinical trial is performed to evaluate the safety, tolerability, pharmacokinetics, and pharmacodynamics of an antagonist of RIPK2 activity or expression of any one of Formulas I-X in subjects with moderately to severely active Crohn's disease. Eligible subjects are men and women 18 years and older. Optionally, two groups of subjects are selected: (i) subjects having a genotype comprising one or more of rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G; and (ii) subjects lacking the genotype.

Inclusion Criteria: Eligible subjects are men and women 18 years and older. Two groups of subjects are selected: (i) subjects having a genotype comprising one or more of rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G; and (ii) subjects lacking the genotype. Subjects are patients with Crohn's disease of at least 3 months' duration, confirmed at any time in the past by radiography, histology, and/or endoscopy. Female patient of childbearing potential must have a negative highly sensitive serum (beta-human chorionic gonadotropin [b-hCG]) pregnancy test result at screening and a negative urine pregnancy test result at Week 0. Subjects must adhere to the following requirements for concomitant medication for the treatment of Crohn's disease, which are permitted provided that doses meeting these requirements are stable, or have been discontinued, for at least 3 weeks before baseline (Week 0), unless otherwise specified: a) Oral 5-aminosalicylic acid (5-ASA) compounds, b) Oral corticosteroids at a prednisone-equivalent dose at or below 40 milligram per day (mg/day), or 9 mg/day of budesonide, or 5 mg/day beclomethasone dipropionate, c) Antibiotics being used as a primary treatment of Crohn's disease, d) Conventional immunomodulators (that is, azathioprine (AZA), 6-mercaptopurine (6-MP), or Methotrexate (MTX)): participants must have been taking them for at least 12 weeks and at a stable dose for at least 4 weeks before baseline. Subjects who has or had extensive colitis for greater than or equal to (>=) 8 years, or disease limited to the left side of the colon for >=12 years, must either have had a colonoscopy to assess for the presence of dysplasia within 1 year before the first administration of study agent or a colonoscopy to assess for the presence of malignancy at the screening visit, with no evidence of malignancy. Subjects must have active Crohn's disease, defined as a baseline Crohn's Disease Activity Index (CDAI) score of >=220 but <=450.

Experimental (Part I): Placebo. Subjects will receive placebo at Weeks 0, 2, 4, and 6. From Week 8 Placebo-treated subjects who are in clinical response at Week 8 (>=100-point reduction from baseline in Crohn's Disease Activity Index (CDAI) or CDAI <150) will continue to receive placebo every 2 weeks from Week 8 through Week 12. Placebo-treated subjects who are not in clinical response at Week 8 will receive test compound (a compound described herein) 400 mg at Week 8 and then test compound every two weeks from Week 10 through Week 12.

Experimental (Part I): Placebo. Test Compound. Subjects will receive test compound 400 milligram (mg) at Week 0 then 200 mg every two weeks through Week 22.

Experimental (Part II): Placebo. Placebo at Weeks 0, 2, 4, and 8. From Week 12, placebo-treated subjects who are in clinical response at Week 8 (>=100-point reduction from baseline in CDAI or CDAI <150) will continue to receive placebo at Weeks 8, 10, and 12. Placebo-treated subjects who are not in clinical response at Week 8 will receive test compound 150 mg at Week 8 and then test compound 75 mg at Weeks 10, and 12.

Experimental (Part II): Test Compound High Dose. Test compound 400 mg at Week 0 and 200 mg at Weeks 2, 4, 8, and 12.

Experimental (Part II) Test Compound Middle Dose. Test compound 150 mg at Week 0 and 75 mg at Weeks 2, 4, 8, and 12.

Experimental (Part II) Test compound Low Dose. Test compound 50 mg at Week 0 and 25 mg at Weeks 2, 4, 8, and 12.

Primary Outcome Measures: Part I: Change From Baseline in the Crohn's Disease Activity Index (CDAI) Score at Week 8 [Time Frame: Baseline through Week 8]—CDAI will be assessed by collecting information on 8 different Crohn's disease-related variables: extra-intestinal manifestations, abdominal mass, weight, hematocrit, total number of liquid stools, abdominal pain/cramping, use of antidiarrheal drug(s) and/or opiates, and general well-being. The last 4 variables are scored over 7 days by the participant on a diary card. Part II: Change From Baseline in the Crohn's Disease Activity Index (CDAI) Score at Week 8 [Time Frame: Baseline through Week 8]—CDAI are assessed by collecting information on 8 different Crohn's disease-related variables: extra-intestinal manifestations, abdominal mass, weight, hematocrit, total number of liquid stools, abdominal pain/cramping, use of antidiarrheal drug(s) and/or opiates, and general well-being. The last 4 variables are scored over 7 days by the participant on a diary card.

Secondary Outcome Measures: Part II: Clinical Remission at Week 8 as Measured by Crohn's Disease Activity Index (CDAI <150) [Time Frame: Week 8 ]. Part II: Clinical Response at Week 8 as Measured by CDAI (>=100-point reduction from baseline in CDAI or CDAI <150) [Time Frame: Week 8]. Part II: Change in Patient-Reported Outcome (PRO)-2 from baseline at Week 8 [Time Frame: Baseline through Week 8]—The PRO-2 score is the sum of the abdominal pain and stool frequency subscores of the CDAI score. Part II: Clinical remission at Week 8 as measured by PRO-2 (PRO-2 <75) [Time Frame: Week 8]. Part II: Clinical response at Week 8 as measured by PRO-2 (>=50-point reduction from baseline in PRO-2 or PRO-2<75) [Time Frame: Week 8]. Part II: Change in Simple Endoscopic Score for Crohn's Disease (SES-CD) from baseline at Week 12 [Time Frame: Baseline through Week 12]—The SES-CD score is based on the evaluation of 4 endoscopic components (presence/size of ulcers, proportion of mucosal surface covered by ulcers, proportion of mucosal surface affected by any other lesions, and presence/type of narrowing/strictures) across 5 ileocolonic segments. Each endoscopic component is scored from 0 to 3 for each segment, and a total score is derived from the sum of all the component scores (range, 0 to 56).

### Example 7. Phase 1B Clinical Trial

A phase 1B clinical trial is performed to evaluate the efficacy of a compound described herein comprising an antagonist of RIPK2 activity or expression of any one of Formulas I-X in participants with moderately to severely active Crohn's disease that have a genotype comprising one or more of rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G.

Experimental. 10 patients positive for rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G are administered the antagonist of RIPK2 activity or expression. 5-10 patients negative for rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G are administered the antagonist of RIPK2 activity. Patients are monitored in real-time. Central ready of endoscopy and biopsy is employed, with readers blinded to point of time of treatment and endpoints.

Inclusion Criteria: Two groups of subjects are selected: subjects having rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G genotype, and subjects lacking rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G genotype.

Primary Outcome Measures: Simple Endoscopic Score for Crohn's Disease (SESCD), Crohn's Disease Activity Index (CDAI), and Patient Reported Outcome (PRO). If the rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G genotype positive group shows at least 20% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 8. Phase 2A Clinical Trial

A phase 2A clinical trial is performed to evaluate efficacy of an antagonist of RIPK2 activity or expression of any one of Formulas I-X in subjects having a genotype comprising rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G, with moderately to severely active Crohn's disease.

Experimental. 40 patients (drug and placebo groups) positive for rs11564258A, rs2357623A or rs2357623G, rs2066845G or rs2066845C, rs5743289A, rs72796367G, rs6752107A, rs12994997A, rs11741861G, rs9494844C, rs6918329G or rs6918329A, and/or rs7404095G are administered the antagonist of RIPK2 activity or expression or placebo for 12 weeks. An interim analysis is performed after 20 patients from each group are treated at the highest dose to look for 40-50% delta between placebo and treated group in primary outcome (at least 20% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

While preferred embodiments of the present examples have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating an inflammatory, fibrostenotic, or fibrotic disease or condition in a subject, the method comprising administering a modulator of Receptor Interacting Serine/Threonine Kinase 2 (RIPK2) activity or expression to the subject, provided a genotype comprising a single nucleotide polymorphisms (SNP) at rs11564258, rs2357623, rs2066845, rs5743289, rs72796367, rs6752107, rs12994997, rs11741861, rs9494844, rs6918329, or rs7404095, is detected in a sample obtained the subject.

2. The method of claim 1, wherein the genotype comprises two SNPs selected from any two of rs11564258, rs2357623, rs2066845, rs5743289, rs72796367, rs6752107, rs12994997, rs11741861, rs9494844, rs6918329, an rs7404095.

3. The method of claim 1, wherein the genotype comprises three SNPs selected from any three of rs11564258, rs2357623, rs2066845, rs5743289, rs72796367, rs6752107, rs12994997, rs11741861, rs9494844, rs6918329, an rs7404095.

4. The method of claim 1, wherein:
a) the SNP at rs11564258 comprises an “A” or a “G” allele.
b) the SNP at rs2357623 comprises an “A” or a “G” allele;
c) the SNP at rs2066845 comprises a “G” or a “C” allele;
d) the SNP at rs5743289 comprises an “A” or a “G” allele;
e) the SNP at rs72796367 comprises a “G” or an “A” allele;
f) the SNP at rs6752107 comprises a “G” or an “A” allele;
g) the SNP at rs12994997 comprises a “G” or an “A” allele;
h) the SNP at rs11741861 comprises a “G” or an “A” allele;
i) the SNP at rs9494844 comprises an “A” or a “C” allele;
j) the SNP at rs6918329 comprises a “G” or an “A” allele; and
k) the SNP at rs7404095 comprises an “A” or a “G” allele.

5. The method of claim 4, wherein:
a) the SNP at rs11564258 is at position “N” within SEQ ID NO: 1;
b) the SNP at rs2357623 is at position “N” within SEQ ID NO: 2;
c) the SNP at rs2066845 is at position “N” within SEQ ID NO: 3;
d) the SNP at rs5743289 is at position “N” within SEQ ID NO: 4;
e) the SNP at rs72796367 is at position “N” within SEQ ID NO: 5;
f) the SNP at rs6752107 is at position “N” within SEQ ID NO: 6;
g) the SNP at rs12994997 is at position “N” within SEQ ID NO: 7;
h) the SNP at rs11741861 is at position “N” within SEQ ID NO: 8;
i) the SNP at rs9494844 is at position “N” within SEQ ID NO: 9;
j) the SNP at rs6918329 is at position “N” within SEQ ID NO: 10; and
k) the SNP at rs7404095 is at position “N” within SEQ ID NO: 11.

6. The method of claim 1, wherein the modulator of RIPK2 activity or expression comprises an antagonist or a partial antagonist of RIPK2.

7. The method of claim 6, wherein the antagonist or partial antagonist comprises ponatinib, sorafenib, regorafenib, gefitinib, or erlotinib.

8. The method of claim 6, wherein the antagonist or partial antagonist comprises a structure selected from the group consisting of Formula I, Formula II, Formula III, Formula IV, Formula V, Formula VI, Formula VII, Formula VIII, Formula IX, and Formula X.

9. The method of claim 1, wherein the inflammatory, fibrostenotic, or fibrotic disease or condition comprises inflammatory bowel disease, Crohn's disease, perianal Crohn's disease ulcerative colitis, intestinal fibrosis, pulmonary fibrosis, or intestinal fibrostenosis.

10. The method of claim 9, wherein the Crohn's disease comprises ileal Crohn's disease.

11. A method of reducing or ablating activity or expression of Receptor Interacting Serine/Threonine Kinase 2 (RIPK2) in a subject, the method comprising administering a modulator of RIPK2 to the subject, provided a genotype comprising a single nucleotide polymorphisms (SNP) at rs11564258, rs2357623, rs2066845, rs5743289, rs72796367, rs6752107, rs12994997, rs11741861, rs9494844, rs6918329, or rs7404095, is detected in a sample obtained from the subject.

12. The method of claim 11, wherein the genotype comprises two SNPs selected from any two of rs11564258, rs2357623, rs2066845, rs5743289, rs72796367, rs6752107, rs12994997, rs11741861, rs9494844, rs6918329, an rs7404095.

13. The method of claim 11, wherein the genotype comprises three SNPs selected from any three of rs11564258, rs2357623, rs2066845, rs5743289, rs72796367, rs6752107, rs12994997, rs11741861, rs9494844, rs6918329, an rs7404095.

14. The method of claim 11, wherein:
a) the SNP at rs11564258 comprises an “A” or a “G” allele.
b) the SNP at rs2357623 comprises an “A” or a “G” allele;
c) the SNP at rs2066845 comprises a “G” or a “C” allele;
d) the SNP at rs5743289 comprises an “A” or a “G” allele;
e) the SNP at rs72796367 comprises a “G” or an “A” allele;
f) the SNP at rs6752107 comprises a “G” or an “A” allele;
g) the SNP at rs12994997 comprises a “G” or an “A” allele;
h) the SNP at rs11741861 comprises a “G” or an “A” allele;
i) the SNP at rs9494844 comprises an “A” or a “C” allele;
j) the SNP at rs6918329 comprises a “G” or an “A” allele; and
k) the SNP at rs7404095 comprises an “A” or a “G” allele.

15. The method of claim 14, wherein:
a) the SNP at rs11564258 is at position “N” within SEQ ID NO: 1;
b) the SNP at rs2357623 is at position “N” within SEQ ID NO: 2;
c) the SNP at rs2066845 is at position “N” within SEQ ID NO: 3;
d) the SNP at rs5743289 is at position “N” within SEQ ID NO: 4;
e) the SNP at rs72796367 is at position “N” within SEQ ID NO: 5;
f) the SNP at rs6752107 is at position “N” within SEQ ID NO: 6;
g) the SNP at rs12994997 is at position “N” within SEQ ID NO: 7;
h) the SNP at rs11741861 is at position “N” within SEQ ID NO: 8;
i) the SNP at rs9494844 is at position “N” within SEQ ID NO: 9;
j) the SNP at rs6918329 is at position “N” within SEQ ID NO: 10; and
k) the SNP at rs7404095 is at position “N” within SEQ ID NO: 11.

16. The method of claim 11, wherein the modulator of RIPK2 activity or expression comprises an antagonist or a partial antagonist of RIPK2.

17. The method of claim 16, wherein the antagonist or partial antagonist comprises ponatinib, sorafenib, regorafenib, gefitinib, or erlotinib.

18. The method of claim 16, wherein the antagonist or partial antagonist comprises a structure selected from the group consisting of Formula I, Formula II, Formula III, Formula IV, Formula V, Formula VI, Formula VII, Formula VIII, Formula IX, and Formula X.

19. The method of claim 11, wherein the subject has an inflammatory, fibrostenotic, and/or fibrotic disease or condition comprising inflammatory bowel disease, Crohn's disease, perianal CD, ulcerative colitis, intestinal fibrosis, pulmonary fibrosis, or intestinal fibrostenosis, and wherein

20. The method of claim 19, wherein the CD is ileal CD.
